(19) United States
(12) Patent Application Publication
(10) Pub. No.: US 2013/0006225 A1
(43) Pub. Date: Jan. 3, 2013

(54) TWIN-TYPE CANNULA ASSEMBLIES FOR HAND-HELD POWER-ASSISTED TISSUE ASPIRATION INSTRUMENTS

(75) Inventor: Robert L. Cucin, West Palm Beach, FL (US)

(73) Assignee: Rocin Laboratories, Inc., West Palm Beach, FL (US)

(21) Appl. No.: 13/408,567

(22) Filed: Feb. 29, 2012

Related U.S. Application Data
(63) Continuation-in-part of application No. 13/094,302, filed on Apr. 26, 2011.
Application No. 13/094,302 is a continuation-in-part of application No. 12/955,420, filed on Nov. 29, 2010.
Application No. 12/955,420 is a continuation-in-part of application No. 12/850,786, filed on Aug. 5, 2010.
Application No. 12/850,786 is a continuation-in-part of application No. 12/462,596, filed on Aug. 5, 2009.

(51) Int. Cl. A61M 1/00

(52) U.S. Cl. 604/542

(57) ABSTRACT

A power-assisted tissue-aspiration instrument employing a new and improved twin-cannula assembly. The twin-cannula assembly includes: an outer cannula mounted stationary to the front portion of a hand-supportable housing containing an inner cannula reciprocation mechanism, and an inner cannula having an open-end type aspiration aperture. The outer cannula has three groups of outer aspiration apertures about its distal portion. The open-end type aspiration opening of the inner cannula reciprocates back and forth to a mid position between the first group of aspiration apertures, and the third group of outer aspiration apertures, so that vacuum pressure is always delivered to at least 1/2 of one the outer aspiration aperture groups as the inner cannula is reciprocated back and forward within the outer cannula.

### RELATED CASES

This application is a Continuation-in-Part (CIP) of copending application Ser. No. 13/094,302 filed Apr. 26, 2011; which is a CIP of copending application Ser. No. 12/955,420 filed Nov. 29, 2010; which is a CIP of application Ser. No. 12/850,786 filed on Aug. 5, 2010; which is a CIP of application Ser. No. 12/462,596 filed Aug. 5, 2009, and copending application Ser. No. 12/813,067 filed Jun. 10, 2010; wherein each said Application is owned by Rocin Laboratories, Inc., and incorporated herein by reference in its entirety.

### BACKGROUND OF INVENTION

1. Field of Invention

The present invention relates generally to new and improved hand-supportable power-assisted tissue-aspiration instruments, and improved twin-cannula assemblies for use therewith.

2. Brief Description of the State of the Knowledge in the Art

Suction lipectomy, commonly known as liposuction or lipoxheresis, is a well known surgical procedure used for sculpturing or contouring the human body to increase the attractiveness of its form. In general, the procedure involves the use of a special type of curet known as a cannula, which is operably connected to a vacuum source. The cannula is inserted within a region of fatty tissue where removal thereof is desired, and the vacuum source suctions the fatty tissue through the suction aperture in the cannula and carries the aspirated fat away. Removal of fat cells by liposuction creates a desired contour that will retain its form.

U.S. Pat. Nos. 5,348,535; 5,643,198; 5,795,323; 6,346,107; 6,394,973; 6,652,522; 6,761,701; 6,872,199; 7,112,200; 7,381,206; 7,384,417; and 7,740,605 to Applicant, incorporated herein by reference, disclose twin-cannula liposuction instruments which allow the practice of suction lipectomy with an unprecedented level of safety and effectiveness.

Also, US Patent Application Publication Nos. 20110034905 A1 and 201100118542 A1, and WIPO Patent Application Publication No. WO 2011/017517 A1 by Applicant, incorporated herein reference, disclose endoscopically-guided twin-cannula tissue aspiration instrumentation and techniques for safely aspirating visceral fat from a patient's mesentery, for the purpose of treating metabolic syndrome, type-II diabetes and other bariatric disorders.

In Applicant's US Patents cited above, the most conservative twin-cannula design provides a single longitudinal slot in an outer cannula registered with a single aperture in a reciprocating inner cannula. The slot length would have to be sufficient to allow exposure of the inner cannula aperture to the tissues at least some of the time in each back-and-forth reciprocation or “stroke.”

In more aggressive twin-cannula configurations, a larger area of patient's tissue is exposed to aspiration suction or vacuum at each point in time by having one or more longitudinal slots (e.g. three slots arranged at 120 degree angles) formed on the outer cannula, which correspondingly register with one or a series of apertures on the inner reciprocating cannula.

Applicant has also disclosed using insulating PFA coatings on the outer surface of the inner cannula, with one or more coextruded conductors, to implement bipolar electro-cautery about the outer aspiration apertures of the twin cannula assembly. Also, by deliberately varying the stroke of the inner cannula (i.e. the distance of its travel up and down the length of the outer cannula slot, or the rate of its reciprocation), the surgeon is provided with improved control over tissue removal in specific areas during fat tissue aspiration operations.

While the twin cannula assisted liposuction (TCAL) instrument designs described in Applicant's U.S. Patents, supra, offer substantial mechanical advantage over a surgeon's manually stroked single cannula, such designs have suffered from a number of shortcomings and drawbacks, including functional and material and tolerance issues.

### Functional Issues of Prior Art Twin-Cannula Assemblies

When performing a liposuction procedure, the surgeon's primary goal should be to aspirate or remove tissue as rapidly and safely as possible, minimizing anesthesia time, and the amount of any local or general anesthetic agents administered, while having complete control of the tissue removal rate so as to avoid wavy or uneven results (e.g. divots) that require remedial procedures. He or she needs to achieve these somewhat crossed purposes in an environment, wherein average procedure liposuction volumes are increasing with the growing obesity epidemic, and economic pressures are quickly increasing to minimize revisional or secondary procedures.

When using power-assisted twin-cannula assemblies constructed according to Applicant's prior art US Patents identified above, Applicant has observed, along the vacuum tubing between the powered hand-piece and the vacuum source (i.e. suction canister), that without concurrent irrigation, the cannula fills with fat from its tip to its base, until some aspirated fat accumulates in the vacuum tubing near the inner cannula hub, then this accumulation or “bolus” of fat moves en masse down the vacuum tubing into the suction canister, and then this cycle repeats itself over an over again. The motion dynamics of aspirated fat along Applicant's prior art twin-cannula assemblies can be explained as follows. The inner cannula lumen presents the smallest inner diameter of the pathway extending from the tip of the cannula to the vacuum canister, and therefore, is the suction limiting parameter of the tissue aspiration system. Thus, the most fibrous portion of aspirated fat creates a plug at the base of the cannula assembly, then the cannula fills from its base to the tip, with some suction force transmitted through the fat column as it is compacted by the suction, and the tumescent fluid sucked out of it. When the obstruction to vacuum becomes almost complete, eventually the full impact of vacuum suction forces the fat plug down the vacuum tubing into the canister, removing the obstruction, and then the cycle repeats. In summary, less vacuum is transmitted to and effectively applied to tissue because the tubing is partially blocked part of the time, along less fat is to be removed.

It would be preferable and more ideal to have the fat aspirated continuously in an even fashion without these build-ups and releases, as a greater degree of vacuum would be delivered to the inner cannula apertures over time, resulting in a greater amount of fat being removed over the same period of time. A more even rate of fat removal would avoid the hills and valleys in the rate of fat removal, maintain the highest sustained average rate of fat removal, and achieve the steadiest or least varying change to that rate of fat removal.

An additional functionality issue with Applicant's prior art twin-cannula assemblies arises by the requirement of the need to register each inner cannula hole or series of holes with its corresponding outer cannula slot. Applicant's prior art twin-cannula assemblies require that the inner cannula not rotate, but be on a rigidly fixed axis with respect to the outer cannula, with a tolerance of ±1° to assure the patient's tissue surrounding the outer cannula is exposed to the vacuum within the inner cannula. This stationary stroke axis imposes design constraints requiring a minimal level of complexity and minimal footprint size for a removable mount, and the necessity of a cannula chamber having a door that can be opened and closed. Delivering bipolar cautery to this stationary axis mount further adds to the complexity and the physical footprint of Applicant's conventional twin-cannula tissue aspiration instruments.

An additional performance issue encountered when using Applicant's twin cannula technology arises with physician habits and the moving center of gravity during most liposuction procedures. To date, every power-assisted liposuction device on the market, other than Applicant's twin cannula liposuction instrument design, requires the surgeon to manually reciprocate the instrument grossly through the tissue. This is because a single cannula vibrating 2-5 mm will not simulate a surgeon's 5-10 mm stroke sufficiently to suck in, and avulse, tissue from the patient, such as fat globules from their stalks, for removal from the aspiration area. Single cannula reciprocation as described above, offers a mechanical advantage, but much less than the exceptional level of mechanical advantage provided when an inner cannula is safely and grossly reciprocated with a slotted outer cannula or sheath of Applicant's twin-cannula liposuction instruments, wherein the center of gravity of the hand-piece moves back and forth along its longitudinal axis, as the inner cannula reciprocates.

While Applicant's twin-cannula liposuction instruments automatically reciprocate the aspiration zone along the outer cannula, and allow the surgeon to maintain the outer cannula relative stationary during periods of selected fat removal, it has been observed that the surgeon using twin-cannula instrumentation has a tendency to move the hand-piece back and forth counter to the reciprocation of the inner cannula—something which was to be avoided when performing twin-cannula assisted liposuction (TCAL). Doing, so the surgeon tends to “neutralize” or “work against” the mechanical advantage of the TCAL hand-piece and keeps the inner cannula stationary vis-à-vis the patient, while the outer cannula is being moves back and forth with the physician's manual stroking Consequently, the surgeon must relearn this motion to achieve maximal efficacy and results with twin cannula assisted liposuction (TCAL). While most surgeons are able to learn the proper and effective use of TCAL instruments within a few hours of hands-on training, they can relapse into bad habits if they do not have access to TCAL instruments in facilities where they typically perform surgery. Thus, as the need for this “relearning” and innate tendency to relapse from years performing prior procedures, results in less than optimal aspiration in many surgeons, a solution to this problem is desired to eliminate the possibility of the surgeon “working against” TCAL instrumentation in this fashion entirely. Though having a much faster rate of handpiece reciprocation can eliminate some of this tendency it cannot eliminate all of it as the physician will still be able to and tend neutralize some harmonic of the rate of inner cannula reciprocation, simply by the tendency to maintain a constant center of gravity within his hand which is holding the reciprocating hand piece.

Material and Tolerance Issues when Manufacturing Prior Art Twin-Cannula Assemblies

To implement a typical TCAL instrument design, Grade 316 stainless steel straight cannulas must be manufactured of uniform smoothness, [symbol] , and an inner cannula OD with a tolerance of ±0.0005″ and an inner cannula inner diameter (ID) with a tolerance of ±0.001″. This implies that the outer cannula must be manufactured with an ID having a tolerance of ±0.0005 and an outer cannula OD having tolerance of ±0.001″ and a similar smoothness of [symbol] . Also, a laser weld must position the outer cannula shaft perpendicular to the hub mount with a precision of 90.0°±0.5°.

As the inner cannula is reciprocated to and fro and the means of reciprocation requires some slack or “play” in the x and y axis as it reciprocates along the z axis, it is important that the first portion of the outer cannula which meets the inner cannula, the inside or undersurface of the hub that mounts it to the hand piece, is suitable chamfered and smooth to minimize any binding. A reusable design requires two pieces of like material (e.g. stainless steel) moving against like mater (e.g. stainless steel), and thus entails dangers of binding. Also, upgrading the inner cannula to 420 SS stainless steel, to minimize this problem by providing “Ginza knife” grade stainless on one sliding surface, will result in a trade off, namely: additional expense, and production lead times for non-standard tubings. Interpolating a Delrin or Teflon ring at the base of the outer cannula would simply exchange one more set of tolerancing issues and cost, for another.

Thus, it is desired to replace a tight tolerance with a loose one, an expensive material with a cheaper one, a similar rubbing surface for a dissimilar rubbing surface and an expensive component that may wear out with a cheap one that can be thrown away after each use.

Implementing bipolar RF cautery within a twin cannula assembly design, as disclosed in Applicant's prior art US Patents, also imposes an additional set of tolerance issues. Typically, a DuPont-manufactured PFA coating must be applied to the inner cannula, with a thickness of 0.0013″ and a tolerance of ±0.002″, as its thickness adds to the tolerance stack of the inner cannula OD, and the outer cannula ID, to encroach on the designed 0.003″ spacing between the inner and outer cannulas. While the PFA coating adds lubricity and helps relieves binding concerns, it does however raise a new issue created by the continued friction on the PFA coating creates the possibility of erosion of the PFA coating, and possible defects in electrical insulation between the inner and outer (electrically-conductive) cannulas, which can short the bipolar cautery circuit. In addition producing a PFA coating of uniform thickness frequently requires first applying a thicker coating and then polishing it down in a two-step process to attain the 0.013″+/−0.002″ required. Therefore, it is desired to use less expensive components not requiring costly tight tolerance manufacturing that are disposable, to eliminate considerations of loss of functionality or dysfunction from the wear and tear of usage.

Clearly, there is a great need in the art for new and improved hand-held fat tissue aspiration instruments, and improved twin-cannula assemblies for use therewith, which overcome the shortcomings and drawbacks of prior art apparatus and methodologies.

### OBJECTS AND SUMMARY OF THE PRESENT INVENTION

Thus, it is a primary object of the present invention to provide new and improved twin-cannula assemblies for hand-held power-assisted tissue aspiration instruments, allowing achieve more efficient aspiration, concurrent bipolar hemostasis, and removal of fat tissue from a patient's body, while overcoming the shortcomings and drawbacks of prior art apparatus and methodologies.

Another object of the present invention is to provide such new and improved twin-cannula assemblies that allow fat to be removed at a maximal sustained rate, even and steadily, without periods of build-up and release, and without recourse to concurrent fluid infusions or sumps which introduce their own functional and production disadvantages.

Another object of the present invention is to provide a new and improved twin-cannula assembly for used with a power-assisted hand-piece, and comprising an inner cannula with an open-end type aspiration aperture or opening, and a hollow outer cannula with multiple outer aspiration apertures formed about the distal portion of the hollow outer cannula, and having an outer cannula base portion stationarily connected to the front portion of the hand-supportable housing.

Another object of the present invention is to provide such a new and improved twin-cannula assembly, wherein multiple outer aspiration apertures comprise first, second and third groups of outer aspiration apertures formed about the distal portion of the outer cannula, and wherein the first group of outer aspiration apertures is formed closest to the distal end of the outer cannula, the second group of outer aspiration apertures is formed closest to the proximal end of the outer cannula, and the second group of outer aspiration apertures is formed the first and third outer aspiration apertures.

Another object of the present invention is to provide such a new and improved twin-cannula assembly, wherein during system operation, the cannula drive mechanism causes the open-end type aspiration opening to reciprocate back and forth to a mid position between the first group of aspiration apertures and the third group of outer aspiration apertures, so that vacuum pressure is always delivered to at least ½ of one the outer aspiration aperture groups as the inner cannula is reciprocated back and forward within the outer cannula, cutting off fat being aspirated into said hollow inner cannula lumen, and thereby progressively delivering more suction performance and achieving a scissoring-effect during tissue aspiration operations.

A further object of the present invention is to provide such a liposuction instrument, wherein the in the cannula assembly can be made from disposable plastic material.

An even further object of the present invention is to provide such new and improved twin-cannula assemblies, equipped with a means for effecting hemostasis during tissue aspiration procedure, using bipolar RF-based electro cauterization.

Another object of the present invention is to provide a new and improved tissue-aspiration instrumentation system which comprises a hand-supportable tissue aspiration instrument employing twin-type cannula assembly which can be driven by pressurized air or electricity, and offers substantially improved tissue aspiration characteristics.

Another object of the present invention is to provide an improved twin-cannula assembly having inner and outer cannula components that can be easily changed, and manufactured with inexpensive components, to provide disposable plastic inner cannulas having an inexpensive angio-catheter style construction.

These and other Objects of the present invention will become apparent hereinafter.

### BRIEF DESCRIPTION OF THE DRAWINGS

For a fuller understanding of the objects of the present invention, reference is made to the detailed description of the illustrative embodiments which are to be taken in connection with the accompanying drawings, wherein;

FIG. 1A is a schematic representation of a first generalized embodiment of the tissue aspiration instrumentation system comprising a hand-supportable tissue aspiration instrument having a hand-supportable housing adapted for receiving a length of flexible tubing connected to a vacuum source, and a new and improved twin-cannula assembly having an open-ended inner cannula operably connected to the flexible vacuum tubing, and coupled to a cannula drive mechanism disposed within the hand-supportable housing and powered by an external power source (e.g. electrical power signals, pressurized air-streams, etc), for reciprocating the inner cannula within a stationary outer cannula, with a fenestrated distal portion, releasably connected to the front portion of the hand-supportable housing;

FIG. 1B is a cross-sectional view of the hand-supportable tissue aspiration instrument shown in FIG. 1A, showing its inner cannula being reciprocated relative to the hand-supportable housing, as its hollow inner cannula base portion is reciprocated within the cylindrical (cannula base portion) guide tube, and tissue is aspirated along the inner cannula lumen, through the lumen formed in the inner cannula base portion, through the cylindrical guide tube, through the stationary tubing connector, and along the flexible tubing towards the vacuum source;

FIG. 2 is a perspective view of a first illustrative embodiment of the tissue aspiration instrumentation system schematically depicted in FIGS. 1A and 1B, and shown comprising a hand-supportable tissue aspiration instrument having (i) a hand-supportable housing with a stationary tubing connector provided at the rear of the housing and receiving a length of flexible tubing connected to a vacuum source, and (ii) a twin-cannula assembly having an inner cannula coupled to an electromagnetic-based cannula drive mechanism disposed within the hand-supportable housing and powered by an AC electrical signal power source, while its stationary fenestrated outer cannula is removed from the front portion of the hand-supportable housing, for purposes of illustration;

FIG. 3A is a cross-sectional view of the hand-supportable tissue aspiration instrument shown in

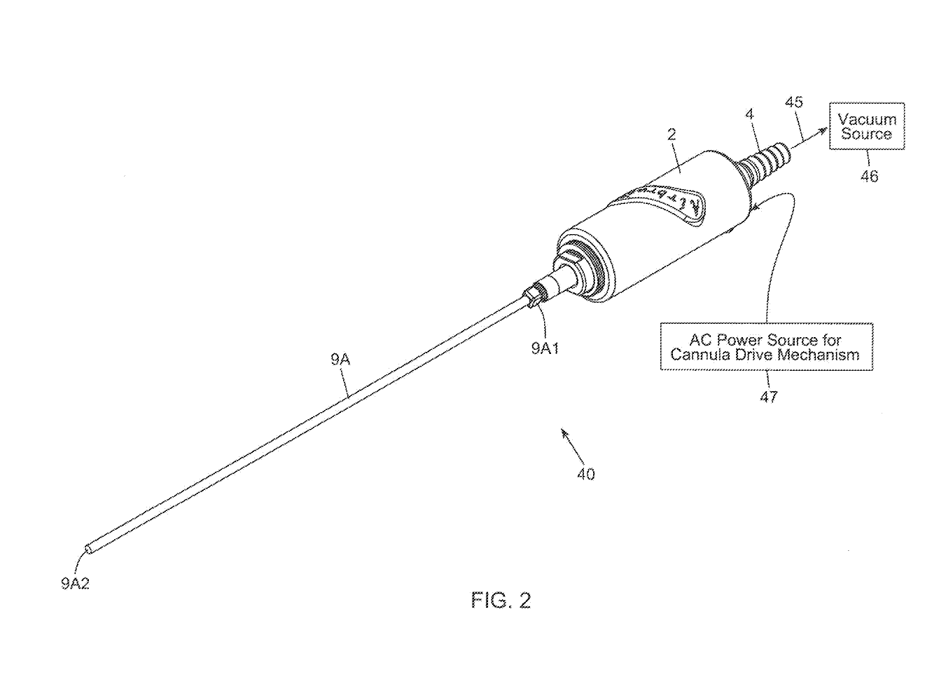

FIG. 2;

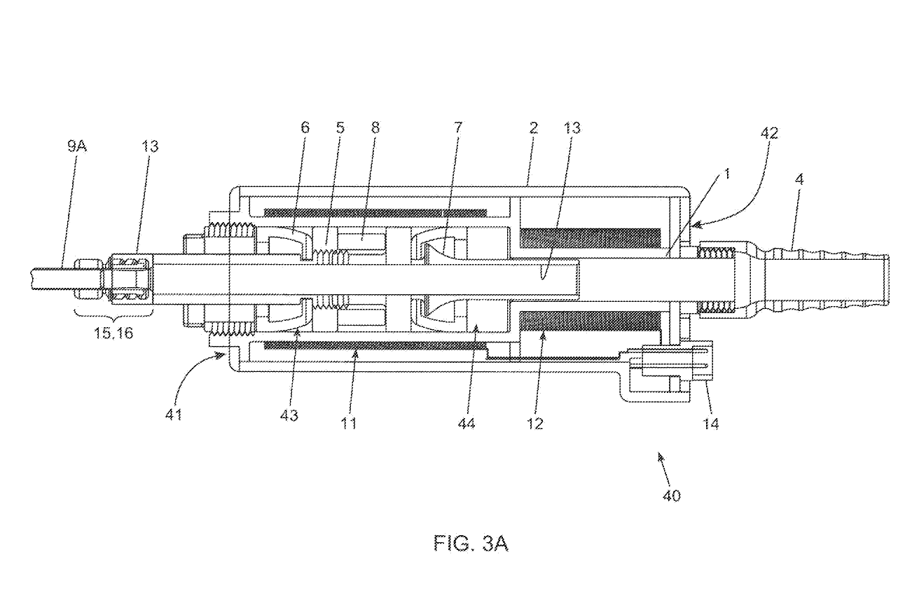

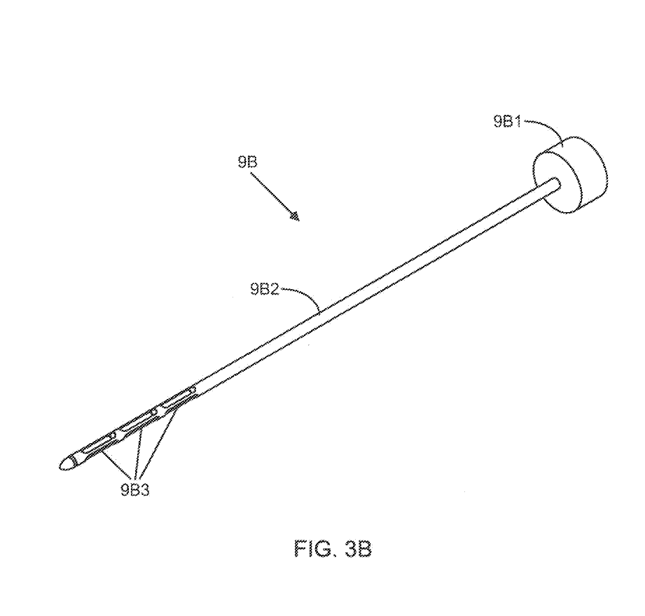

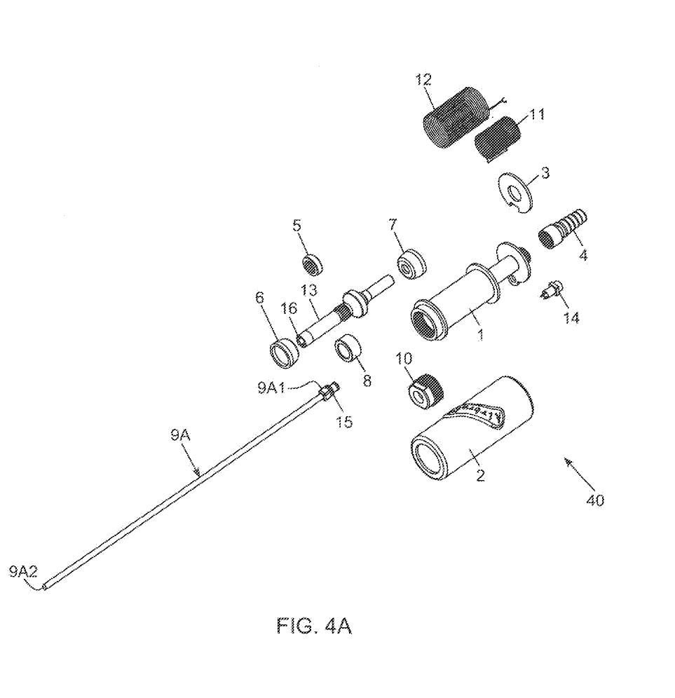

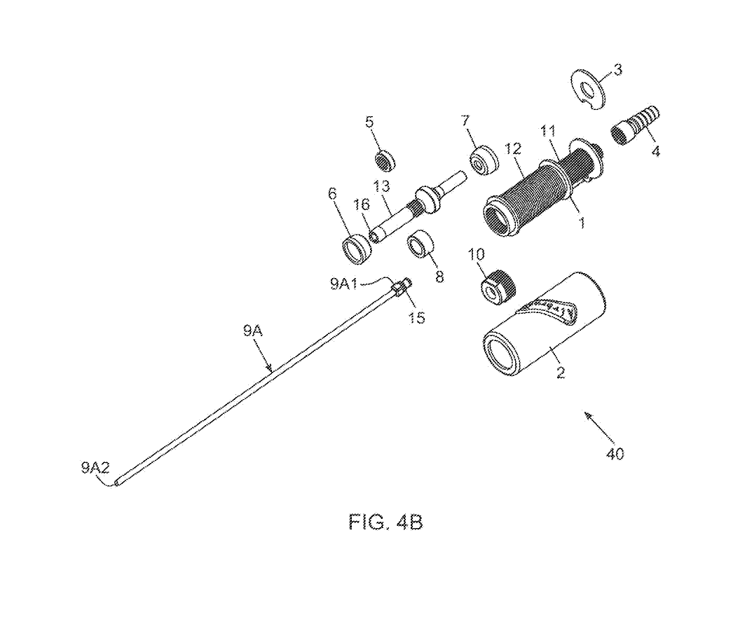

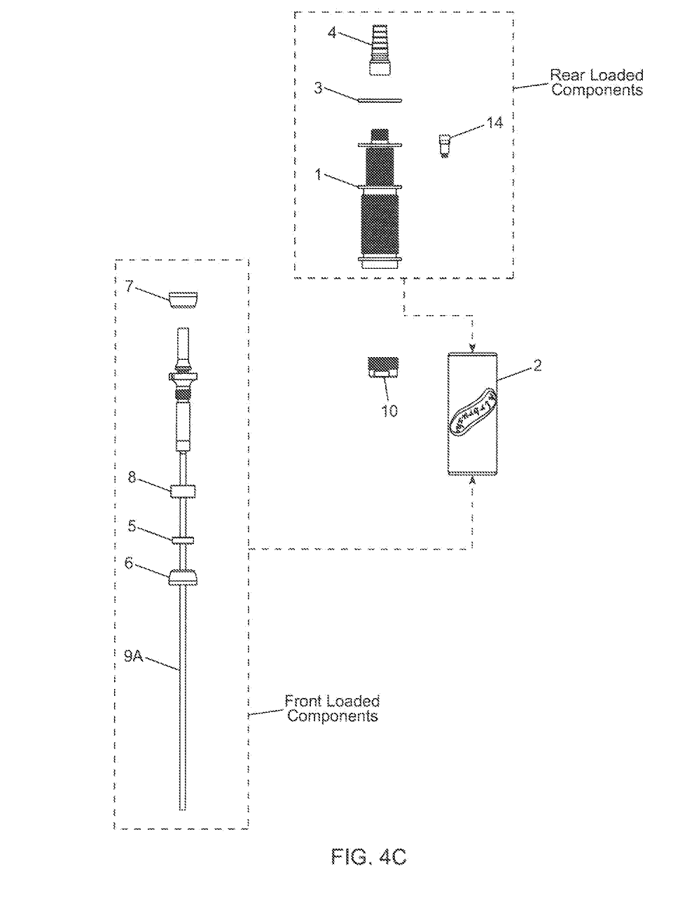

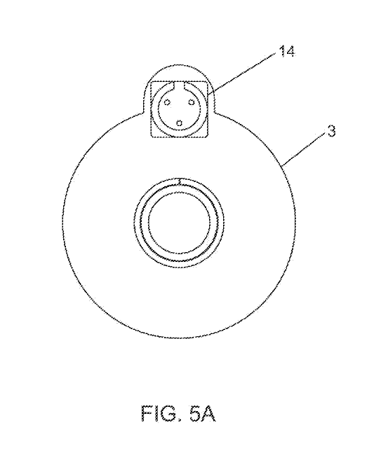

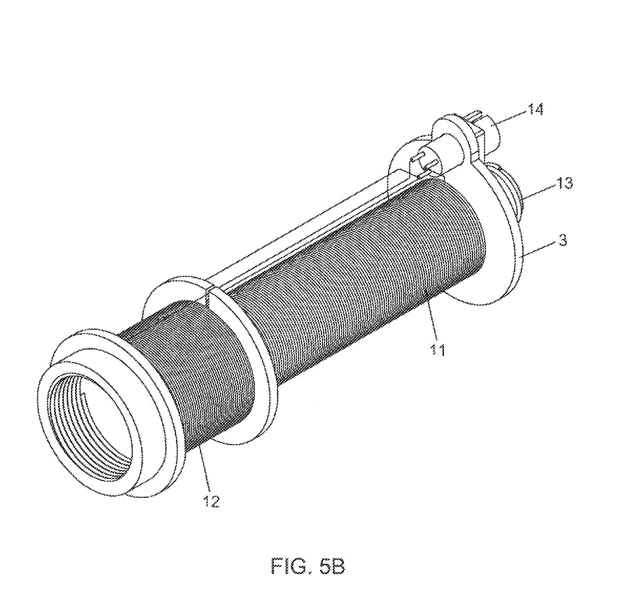

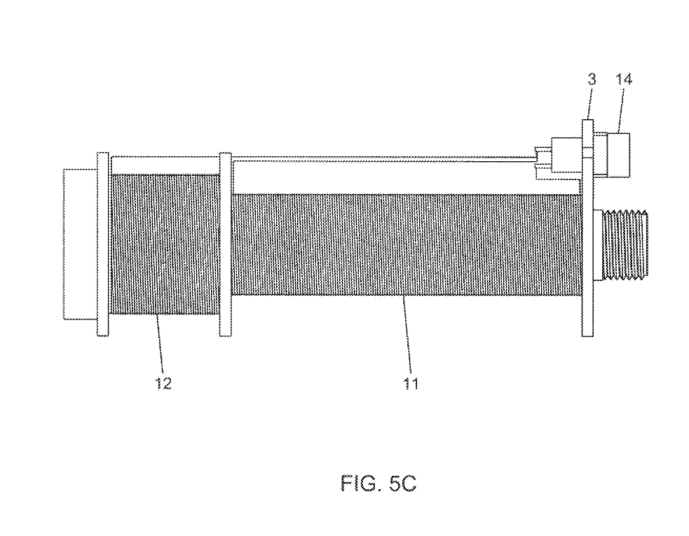

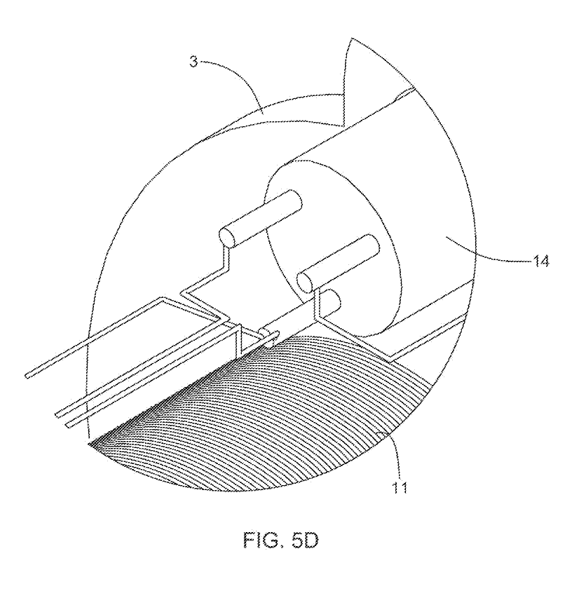

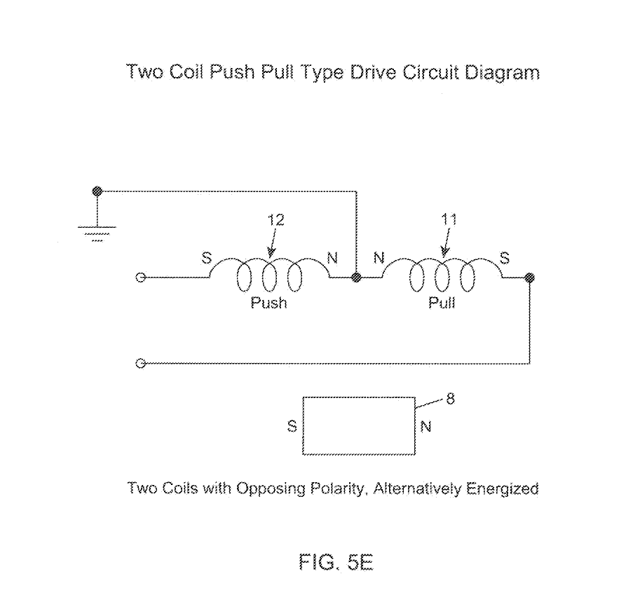

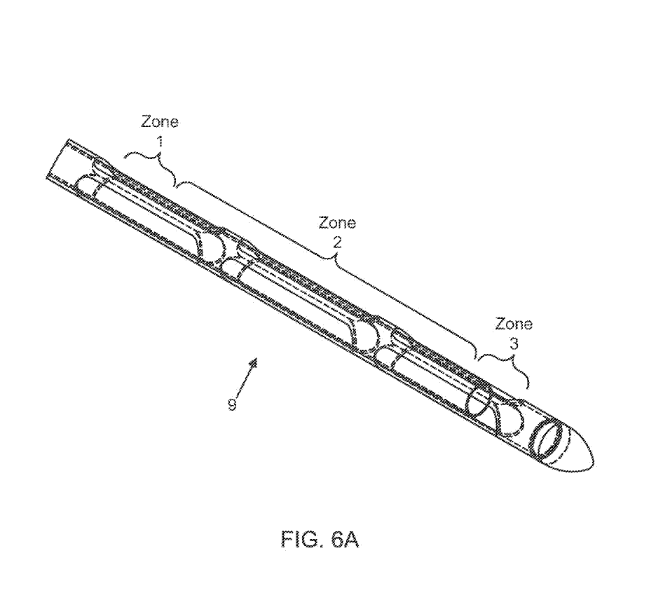

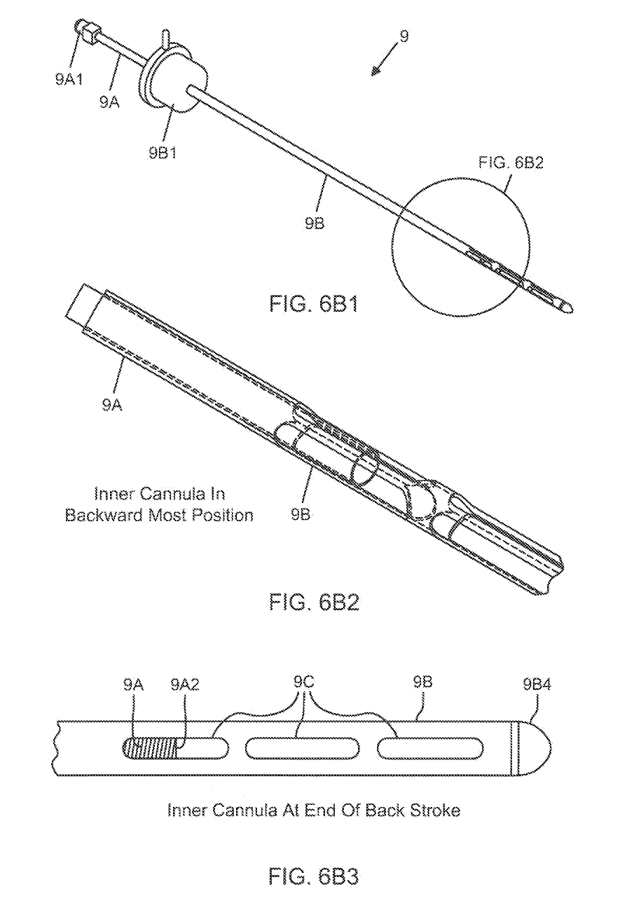

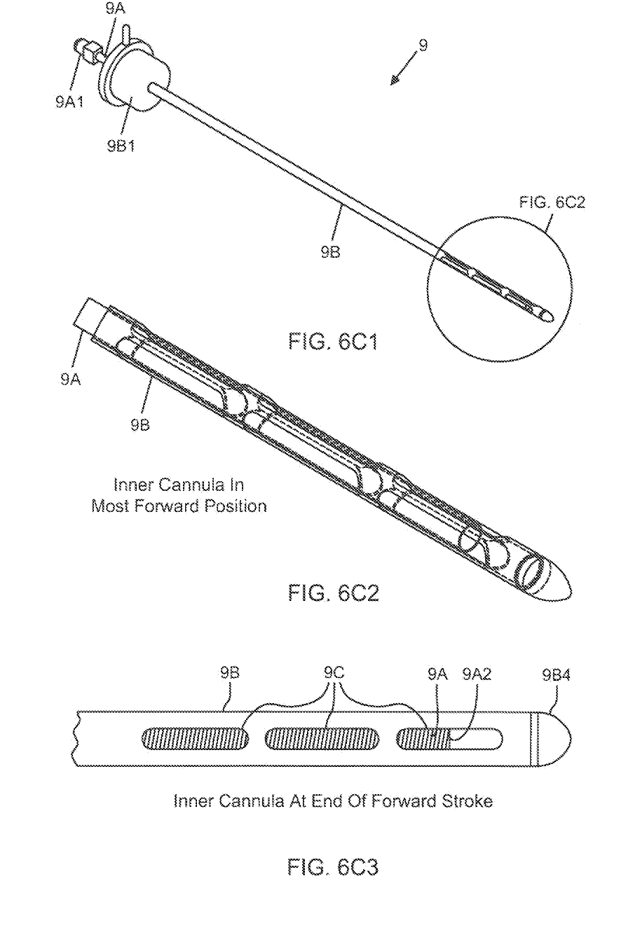

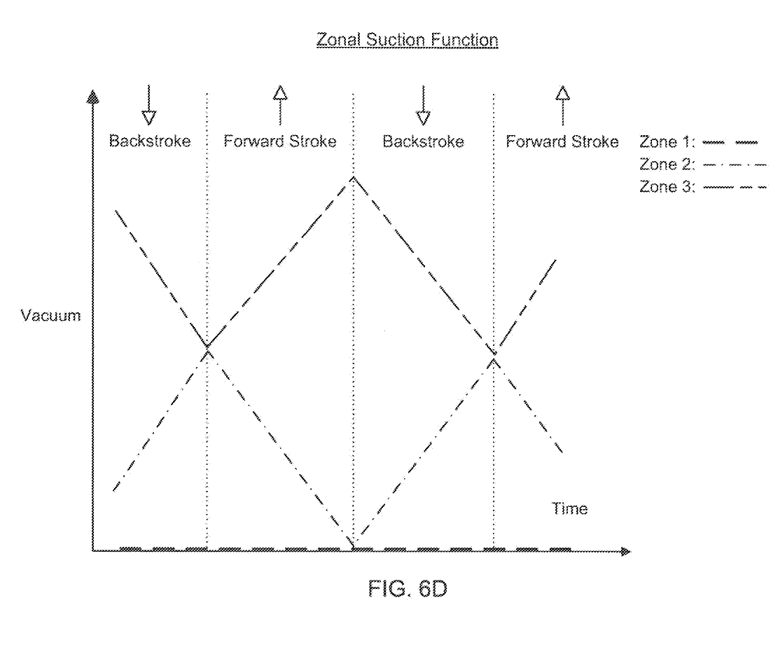

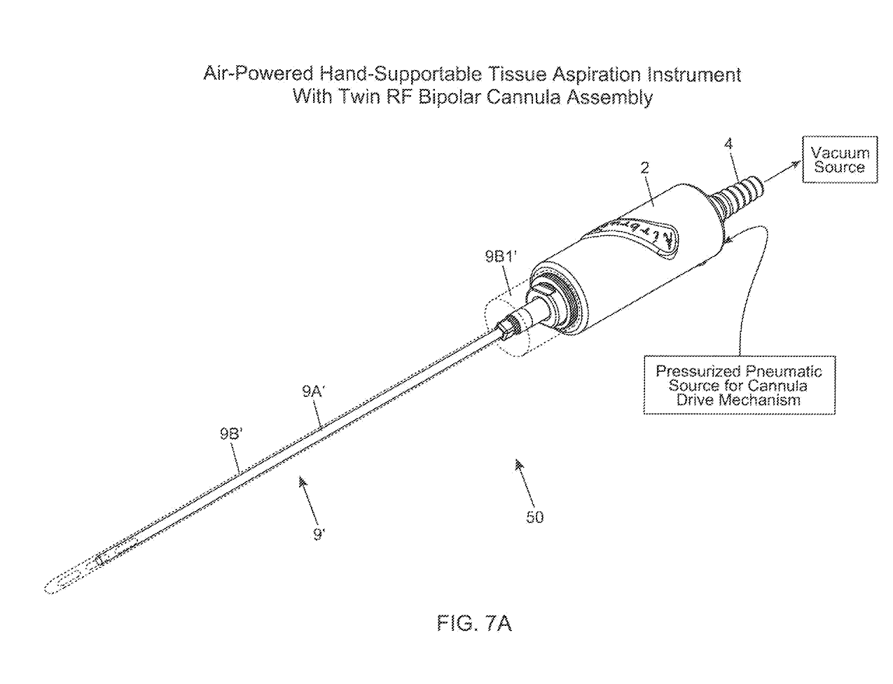

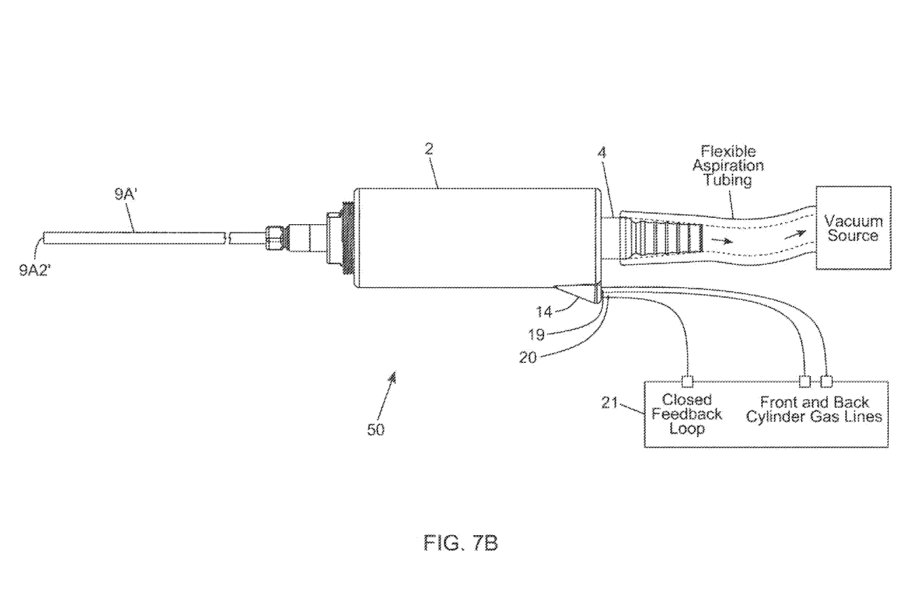

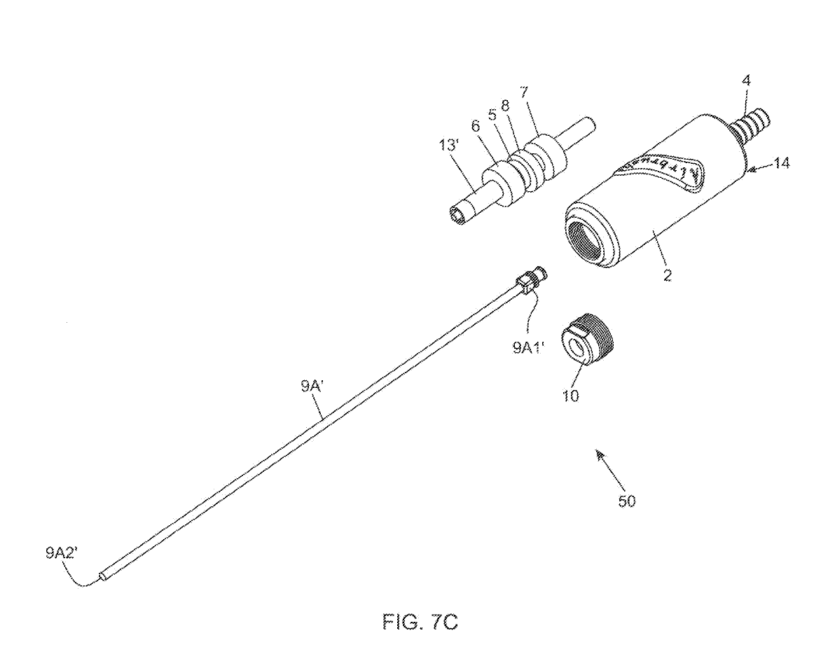

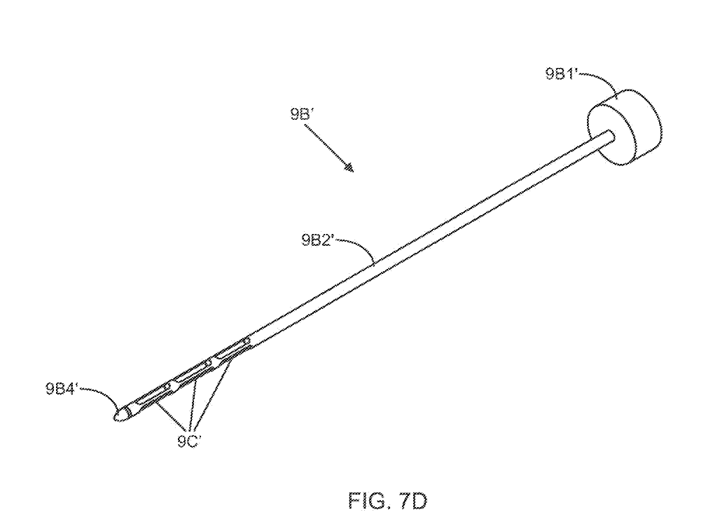

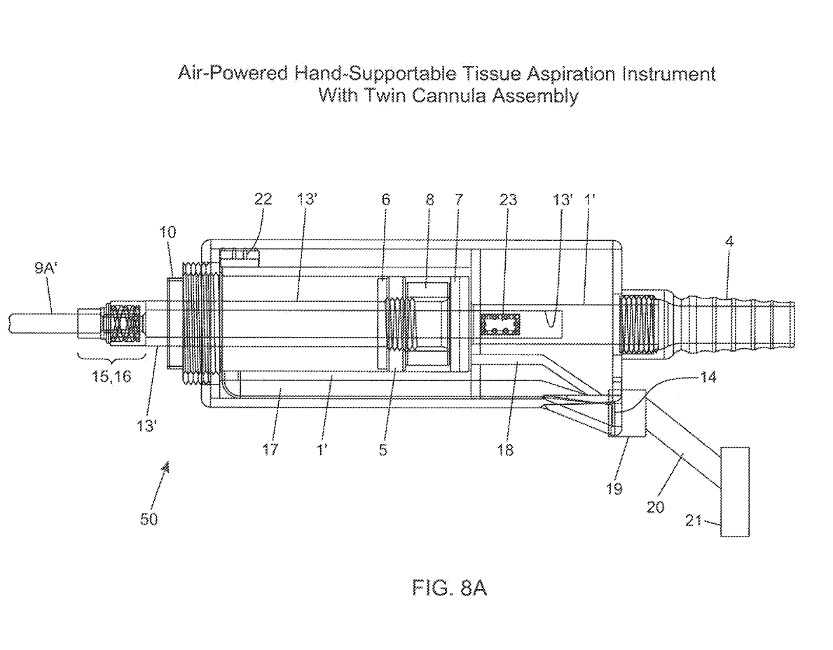

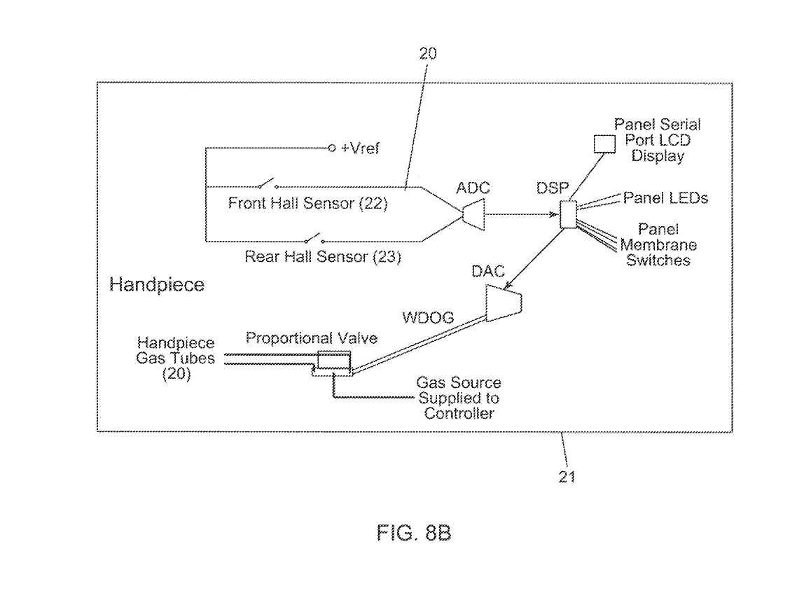

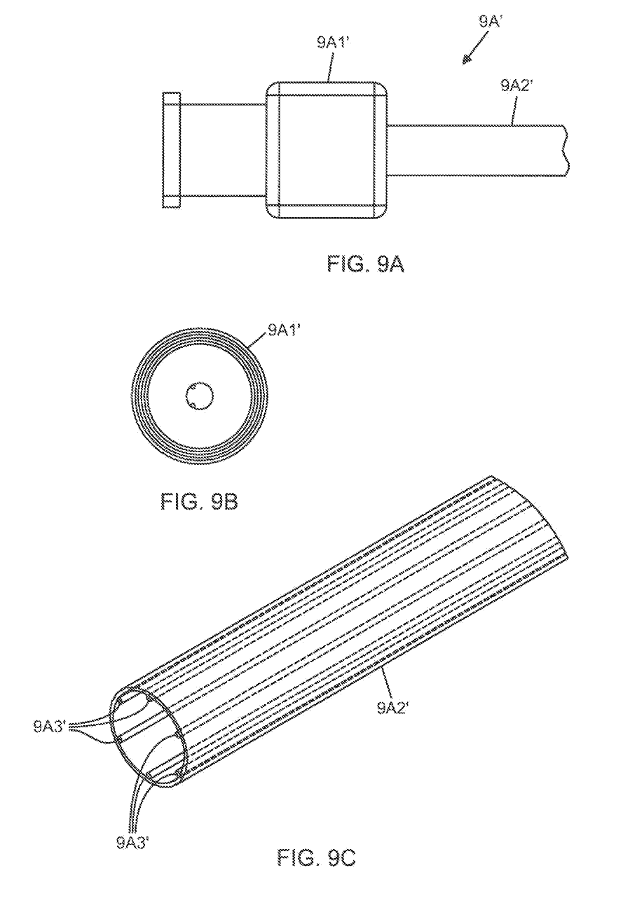

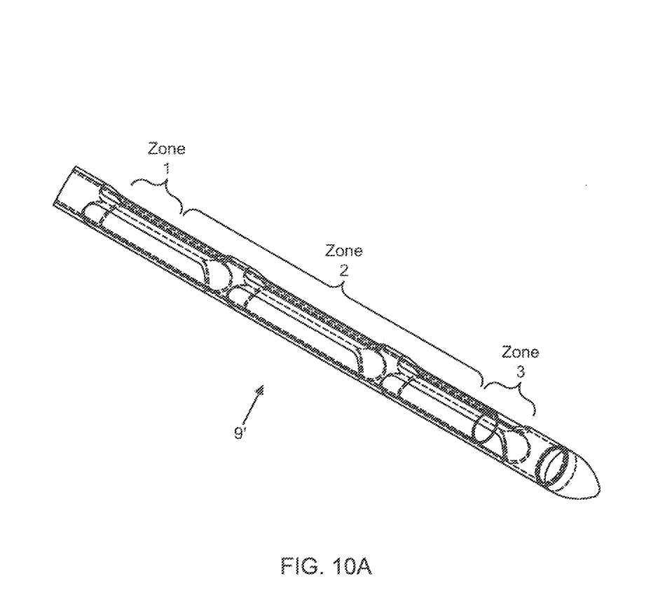

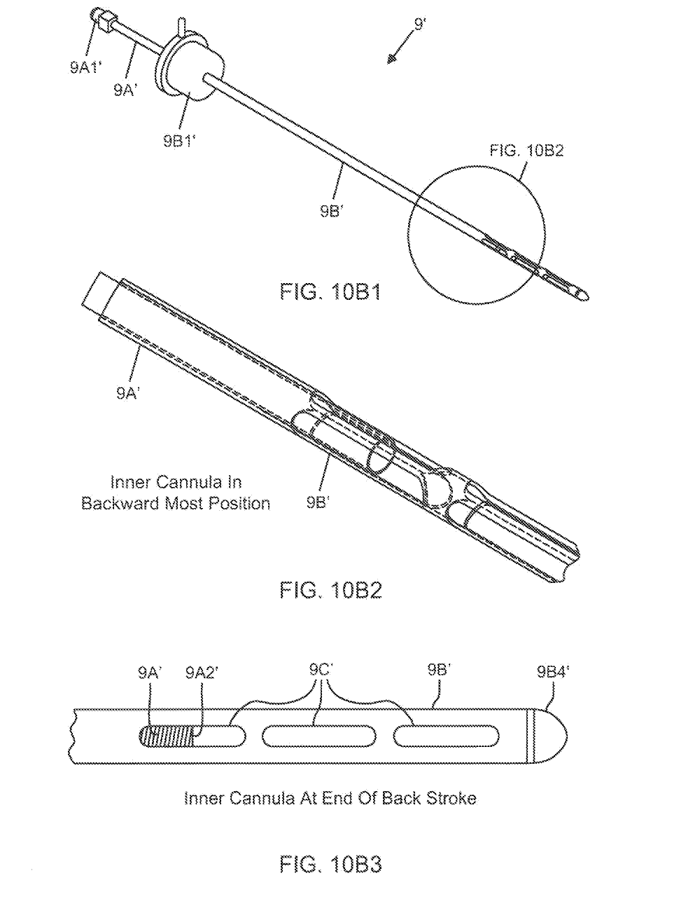

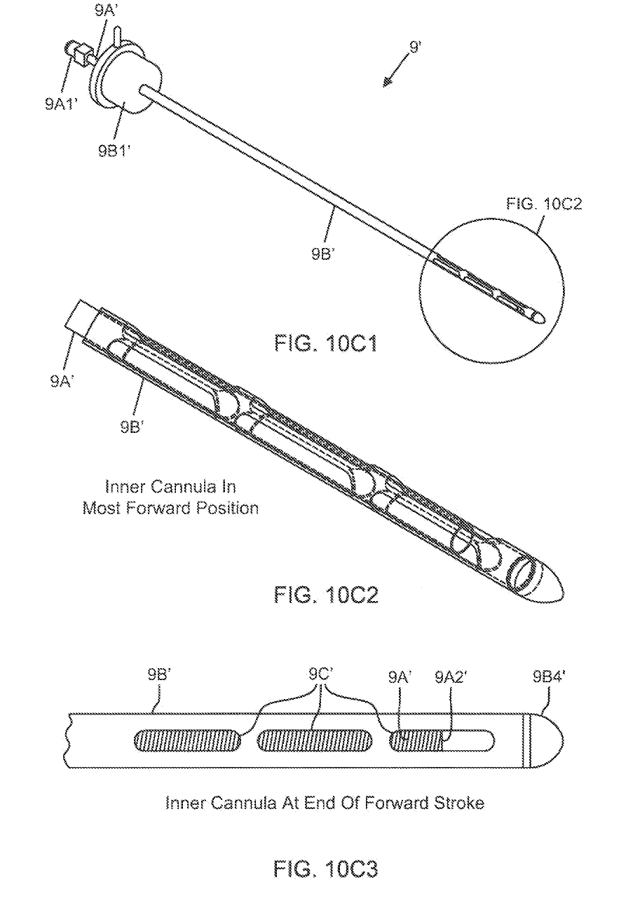

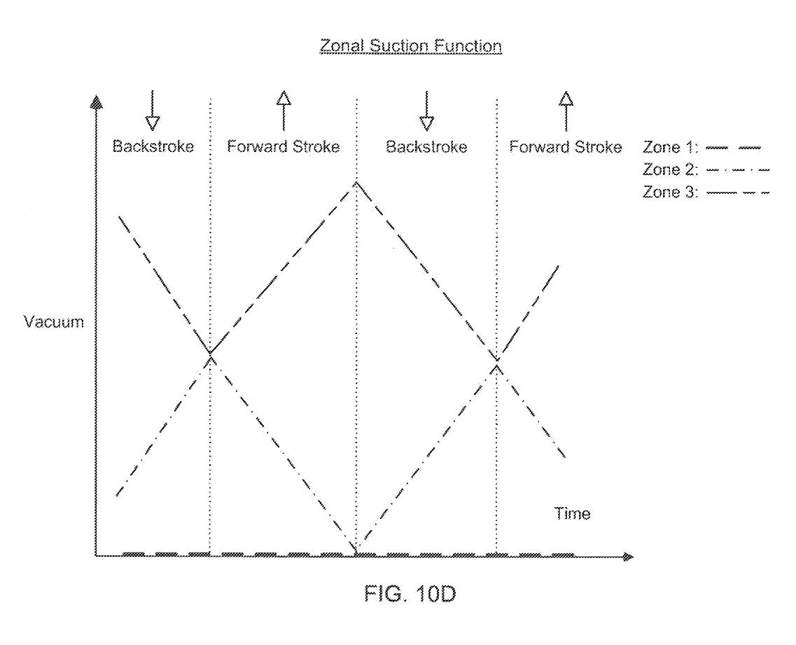

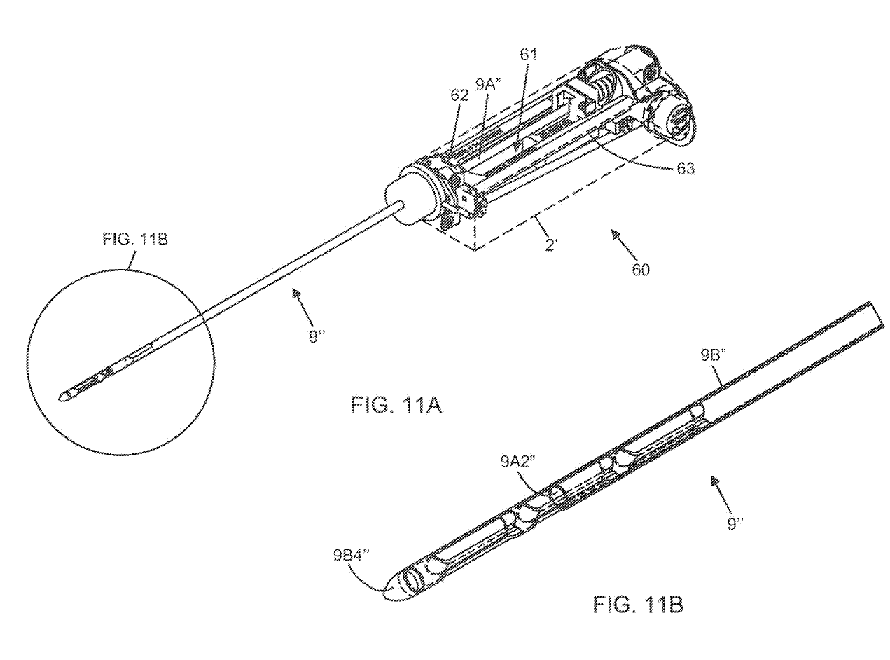

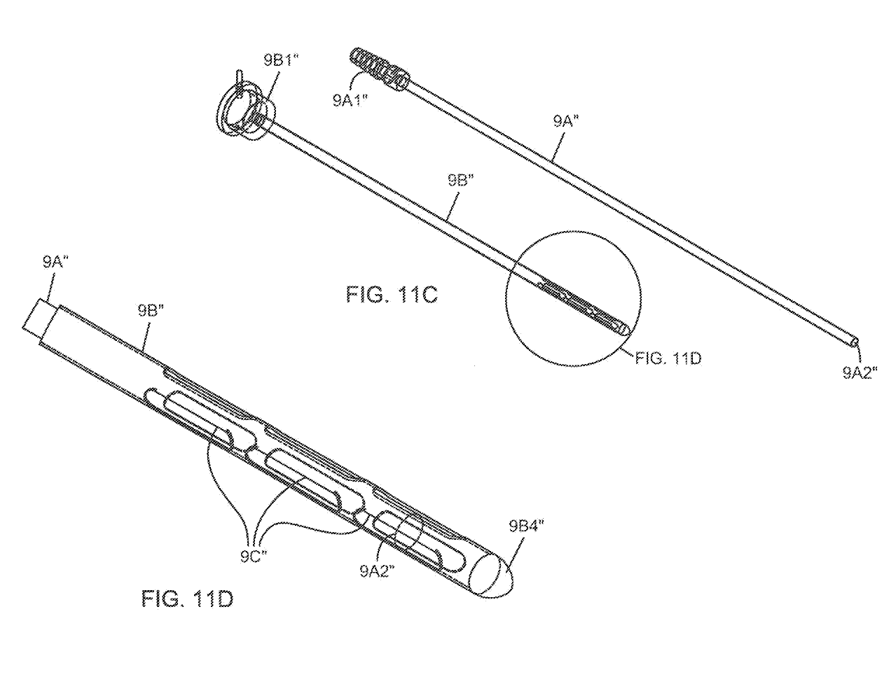

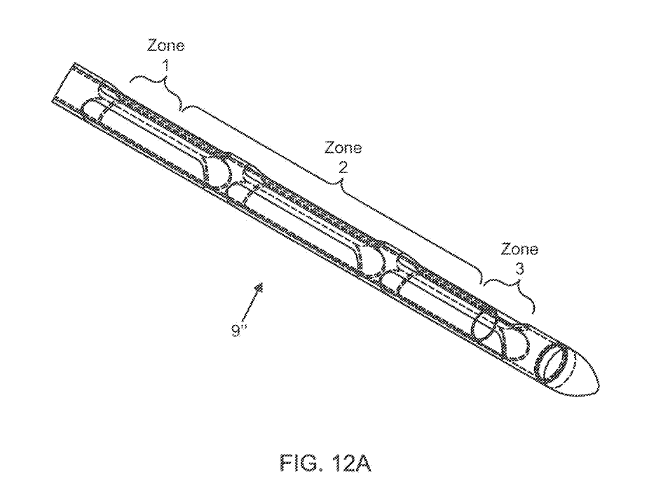

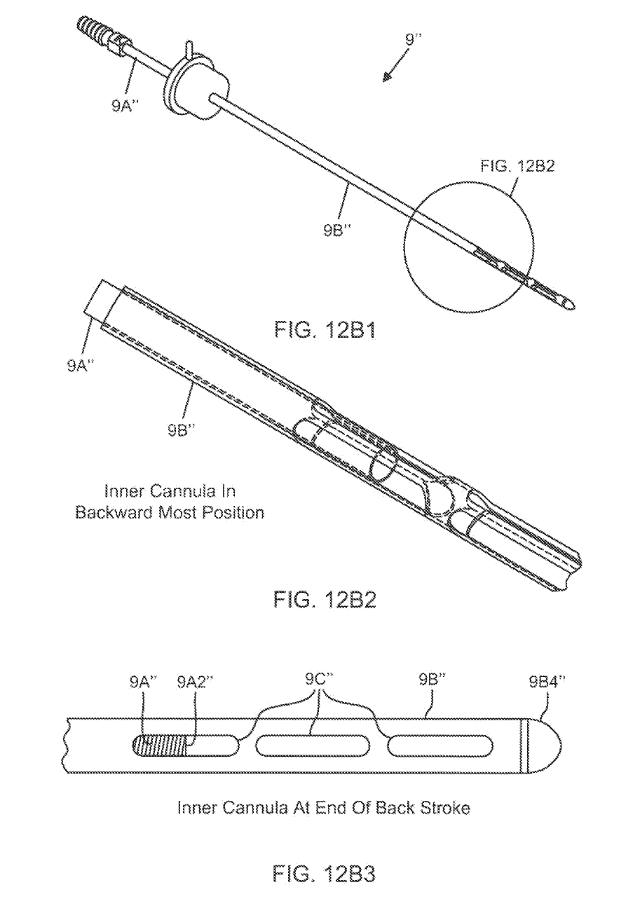

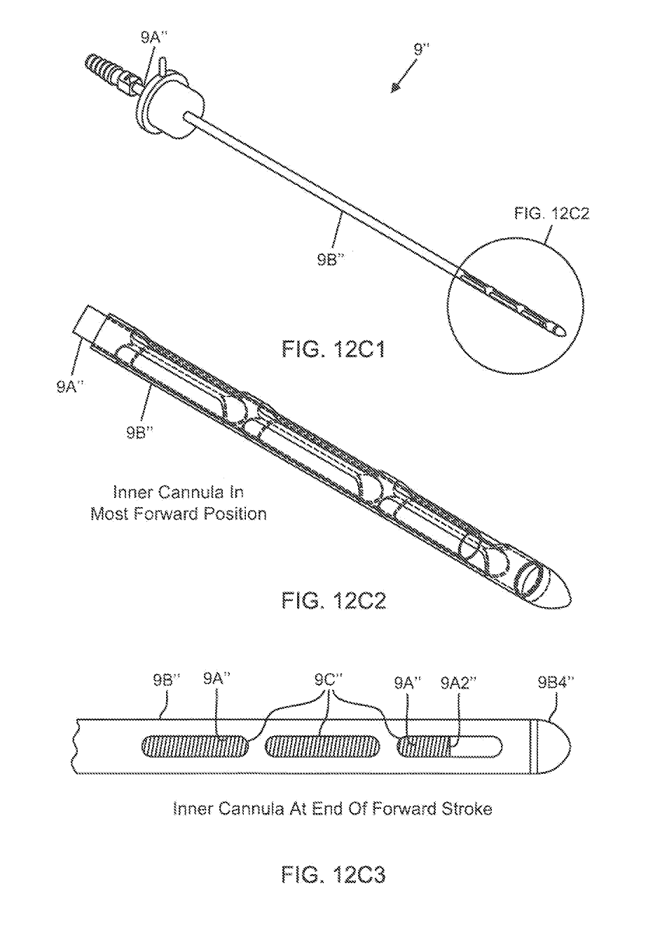

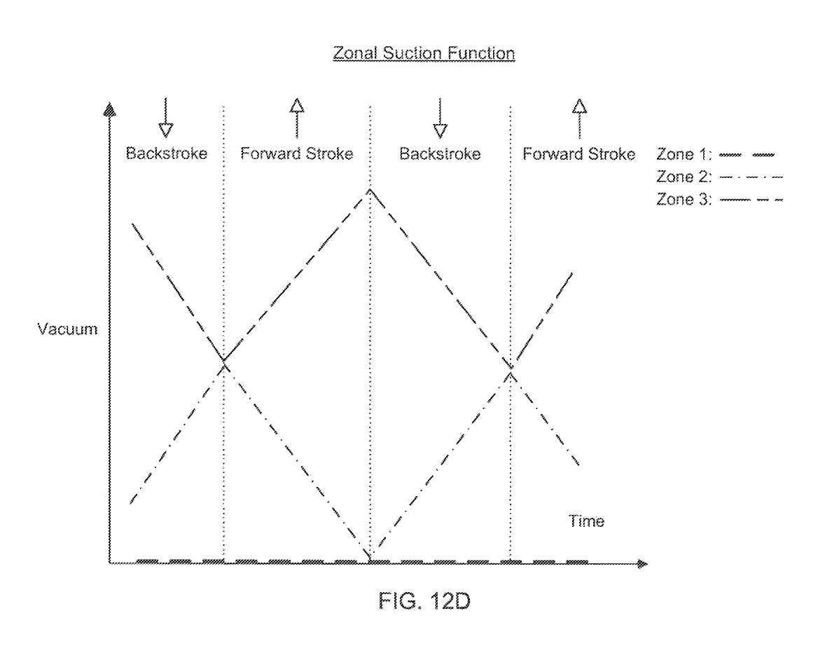

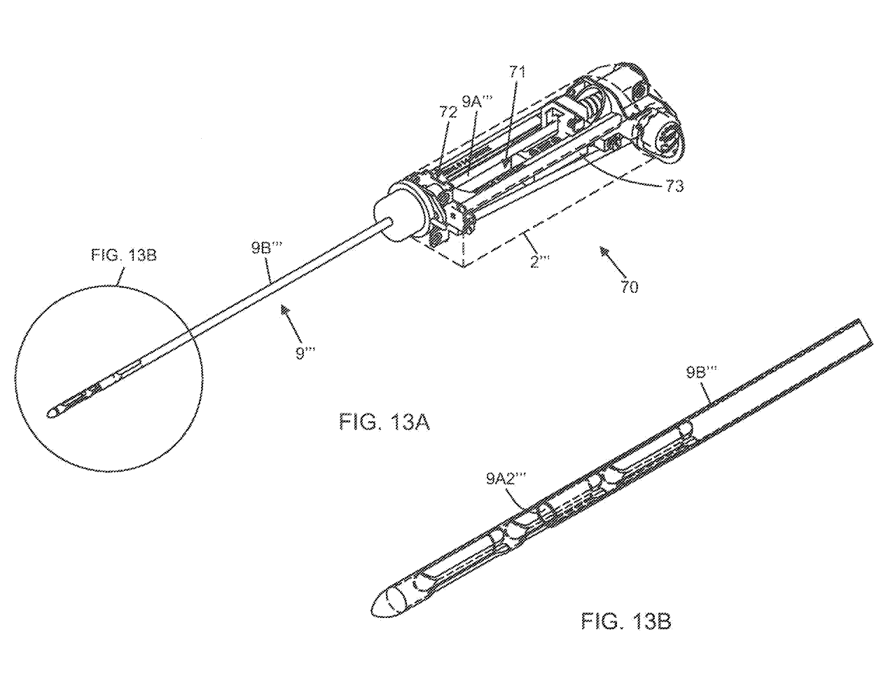

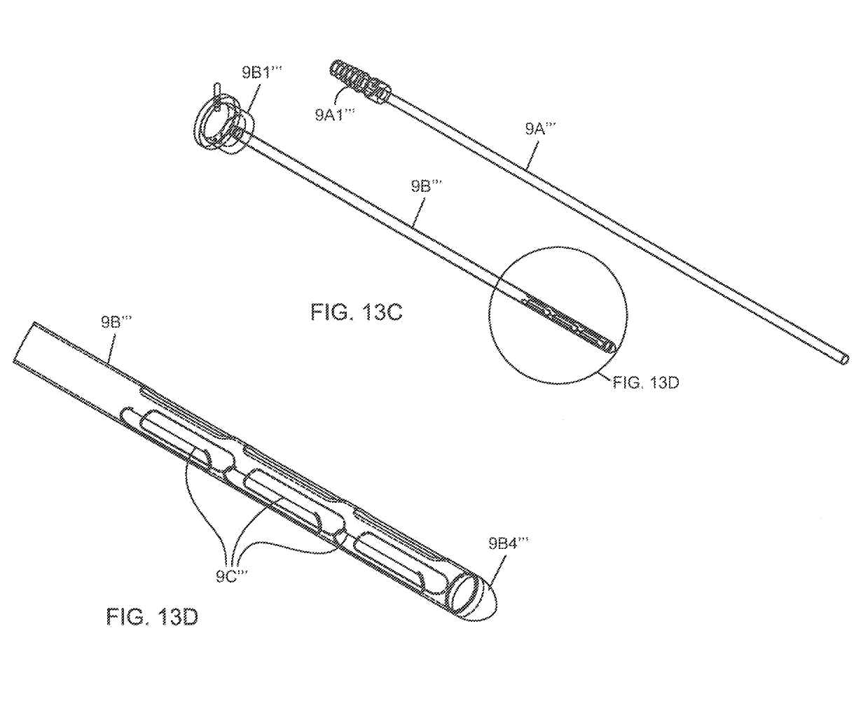

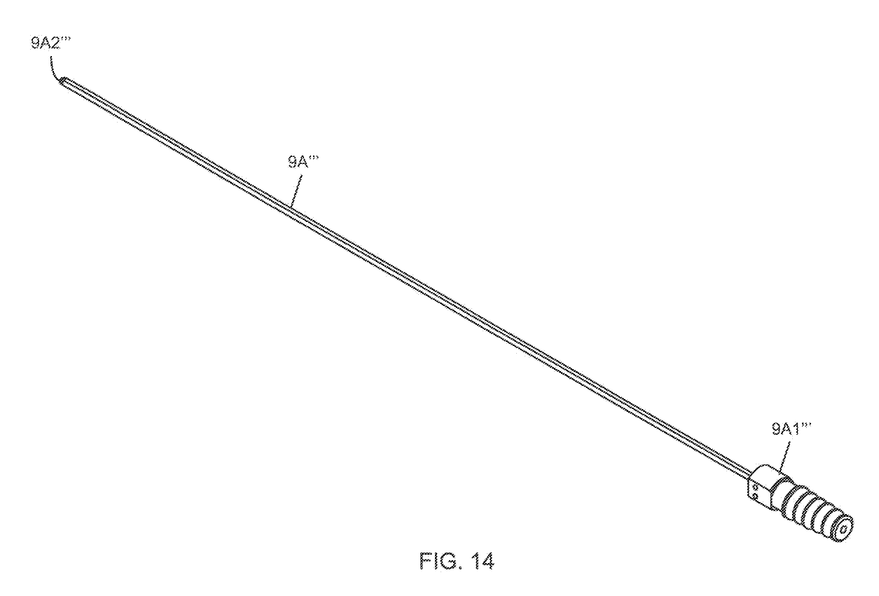

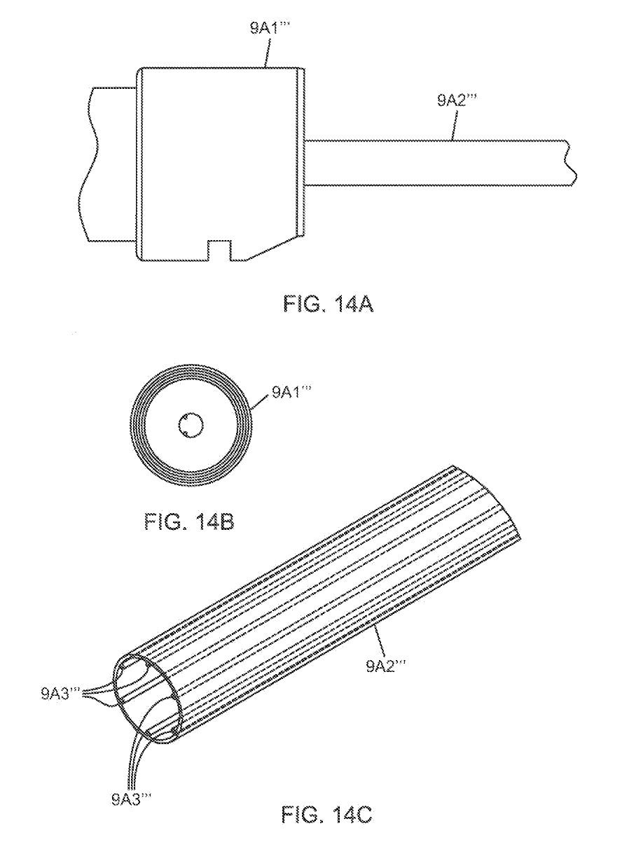

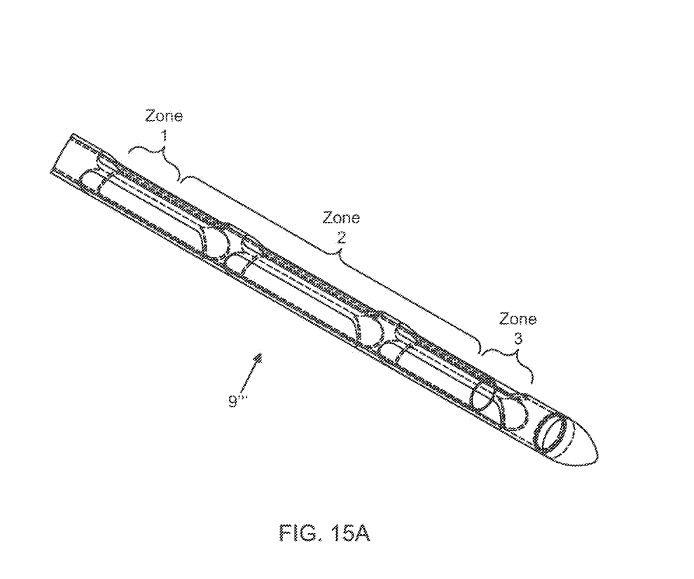

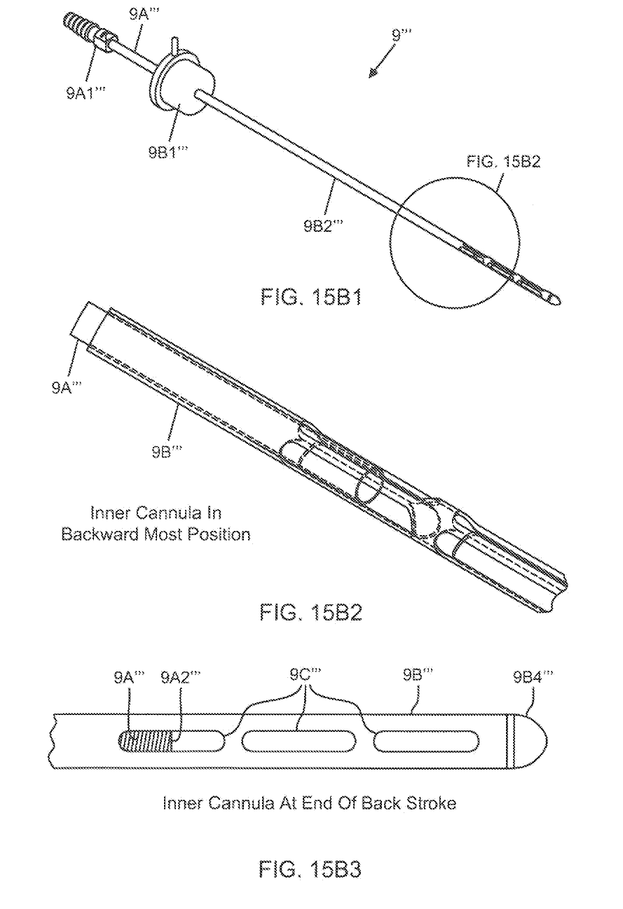

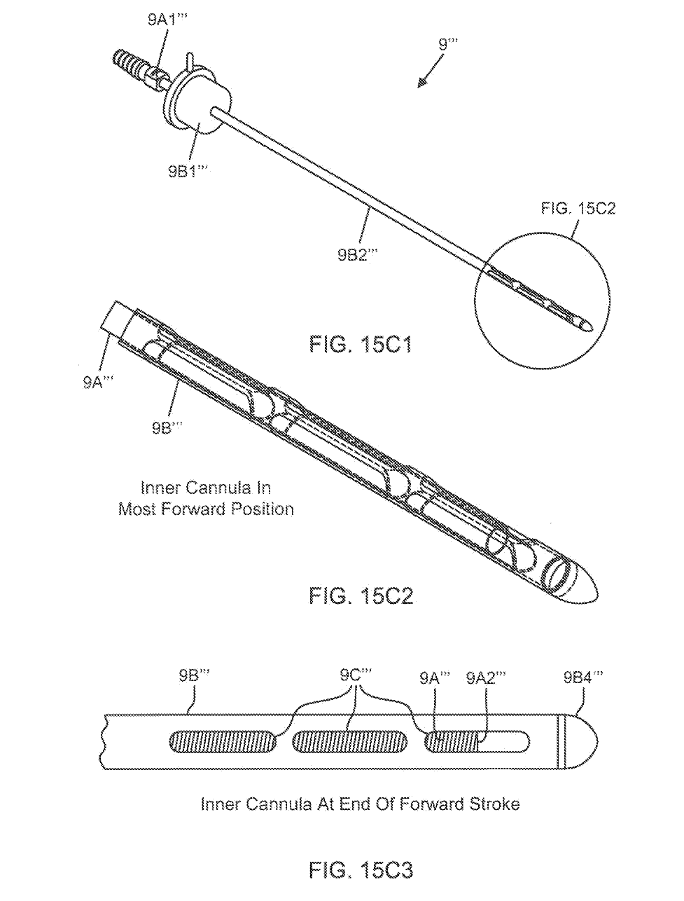

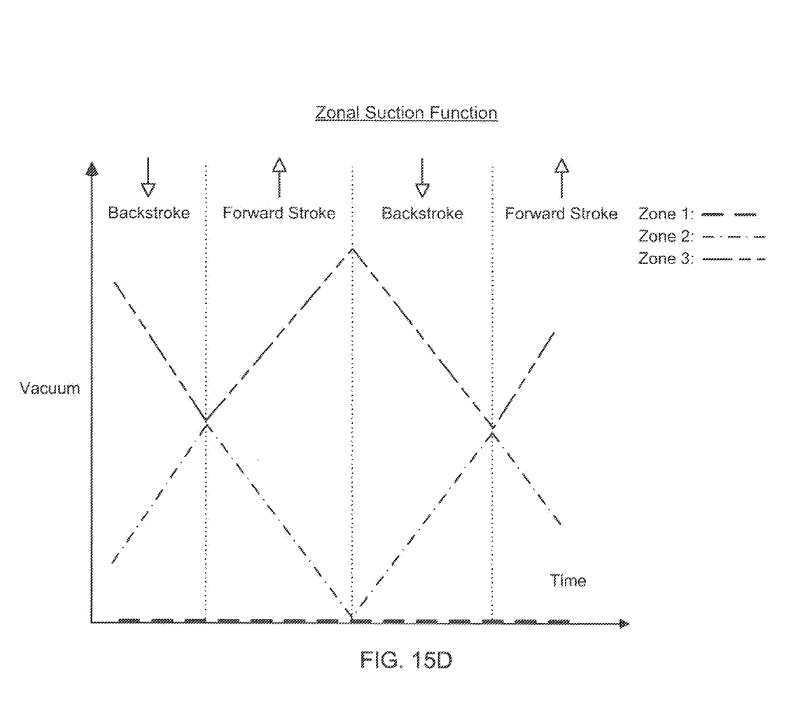

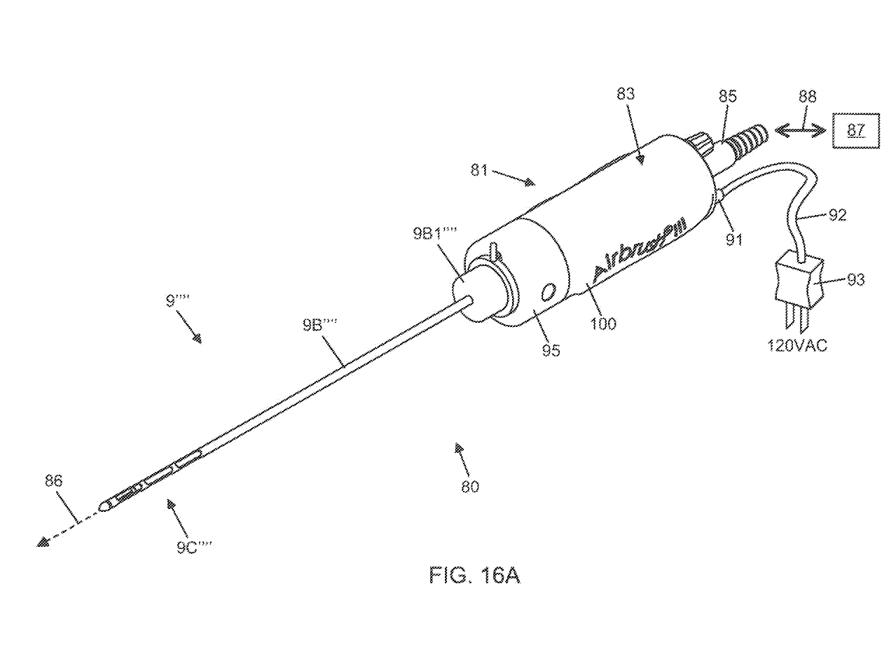

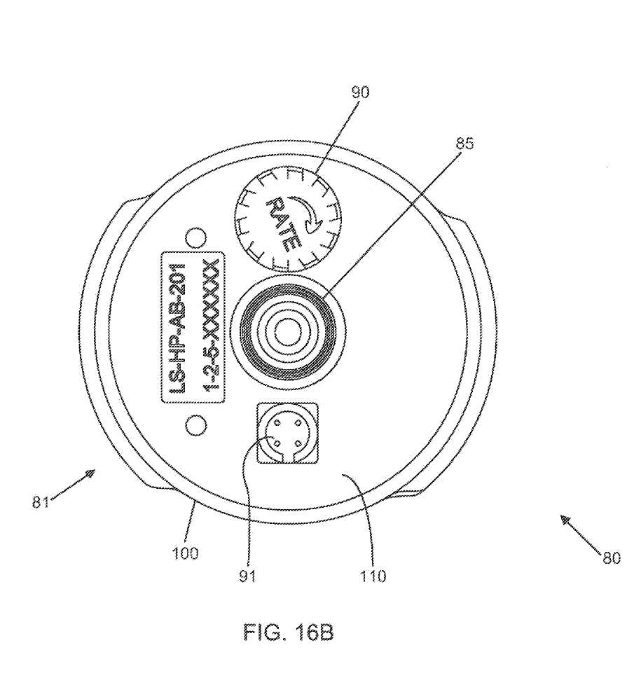

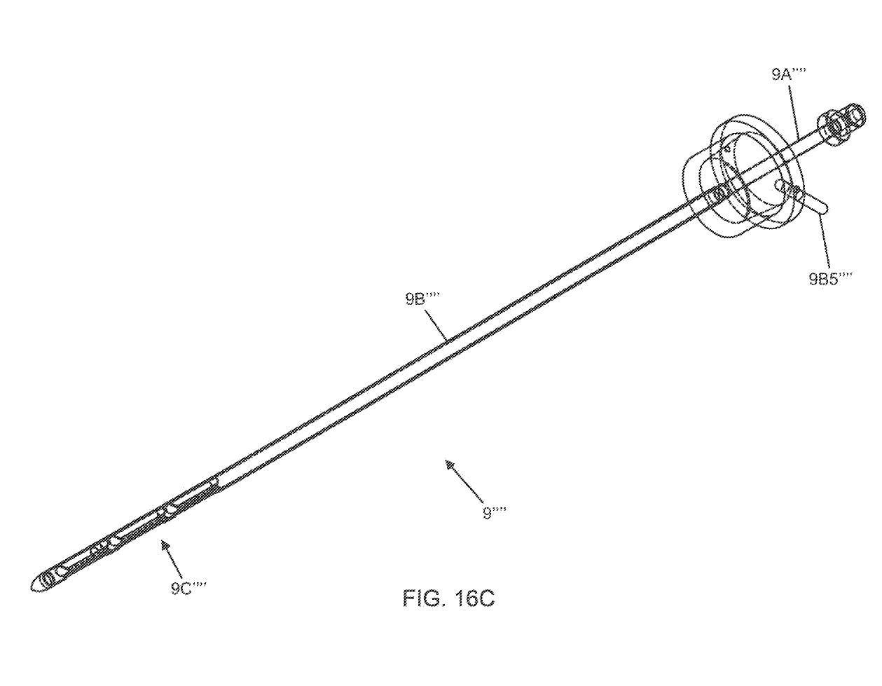

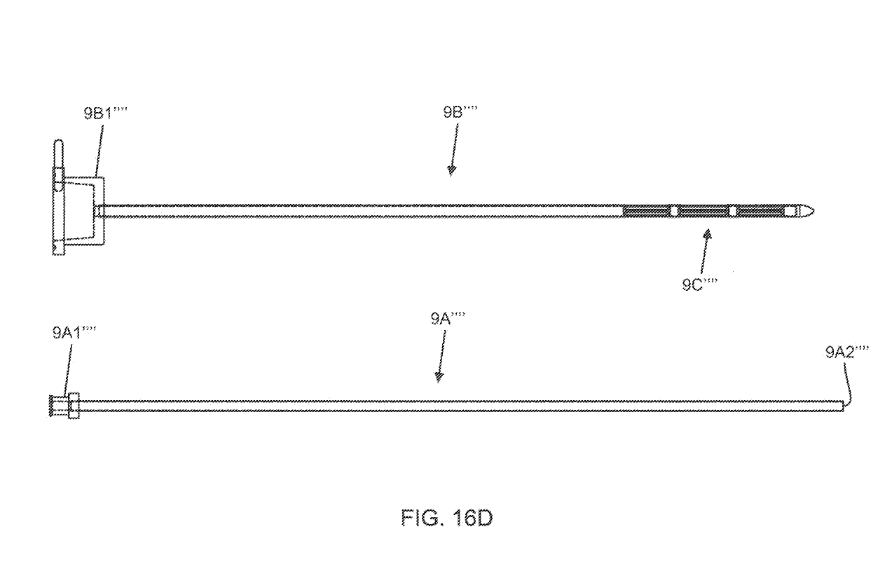

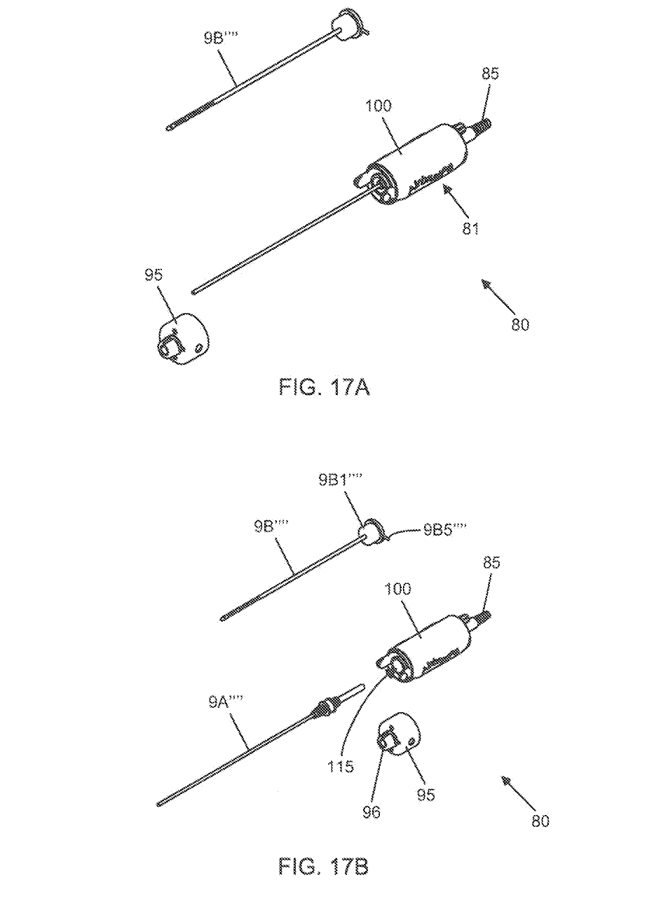

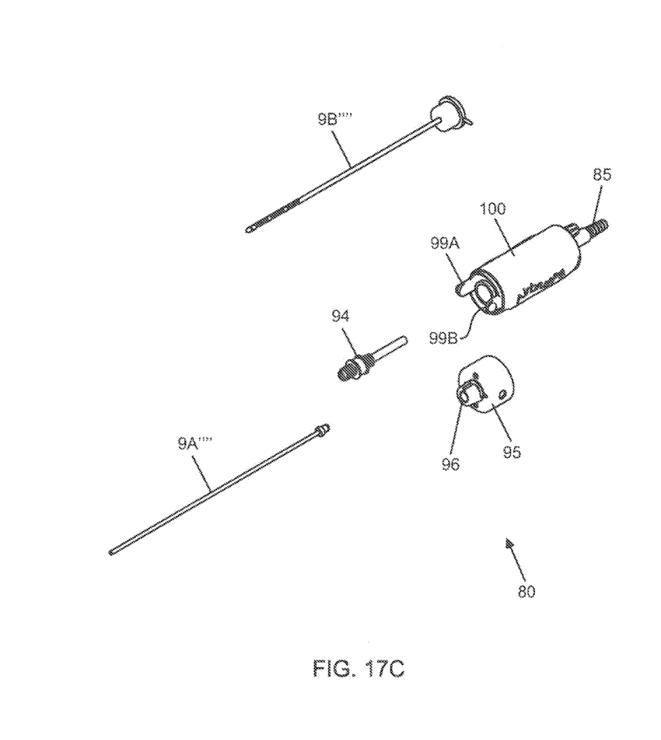

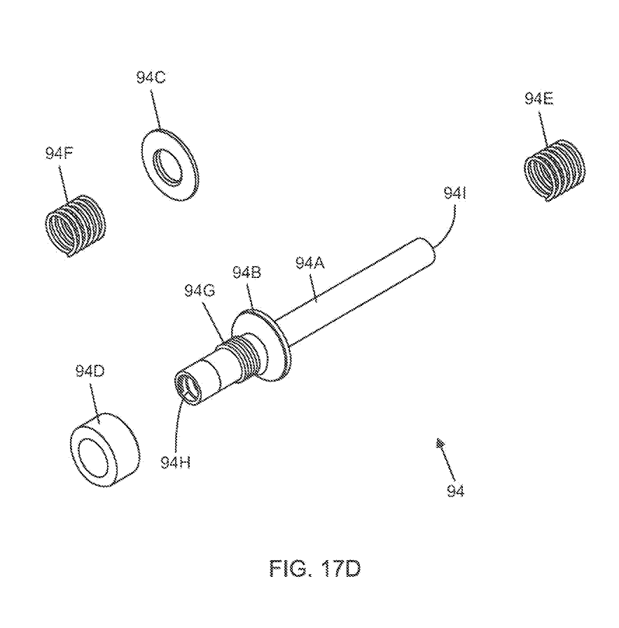

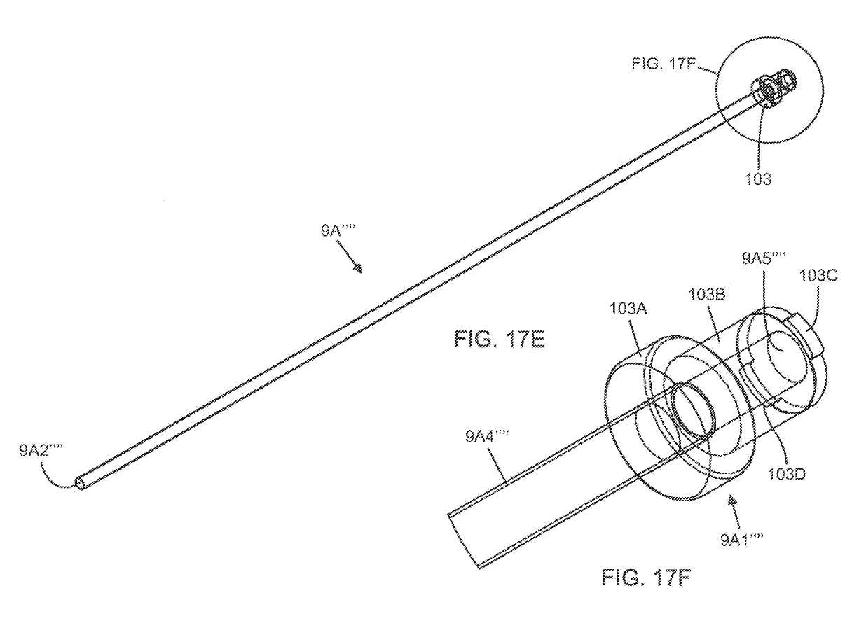

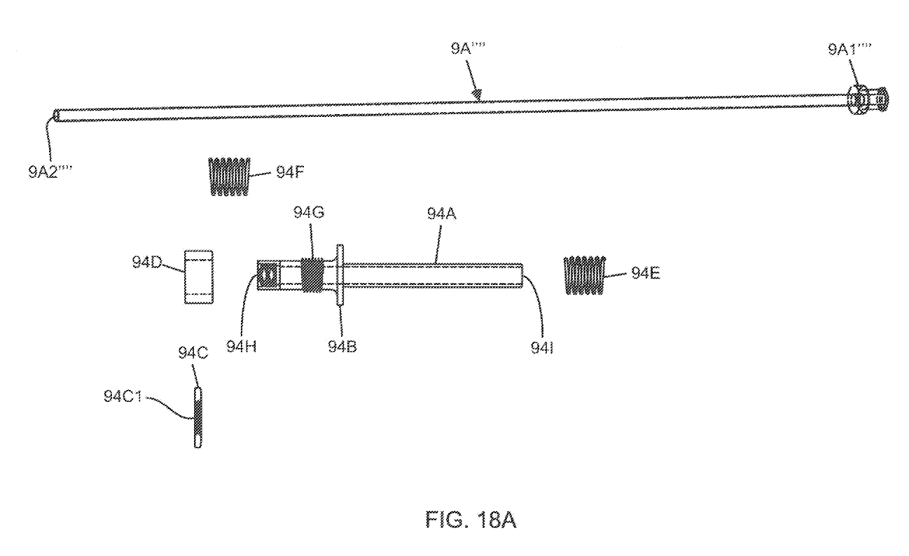

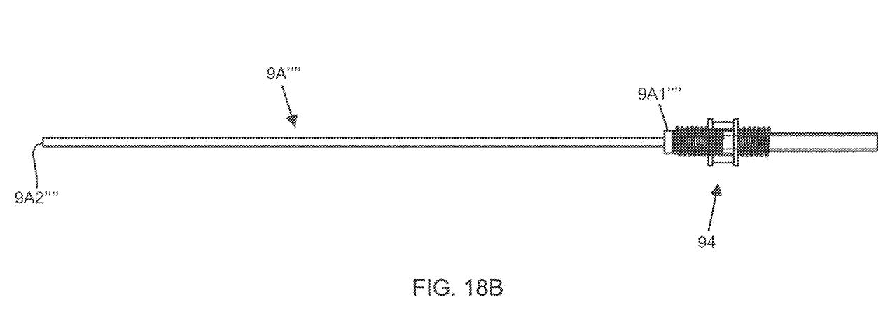

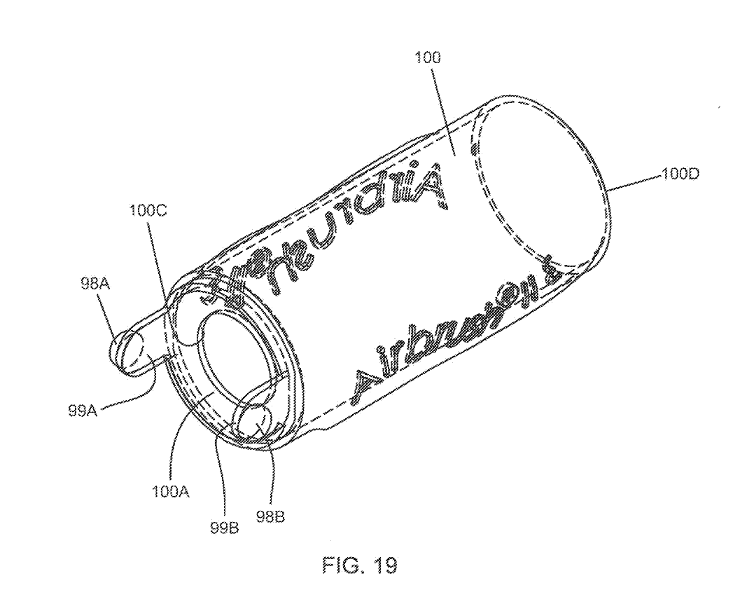

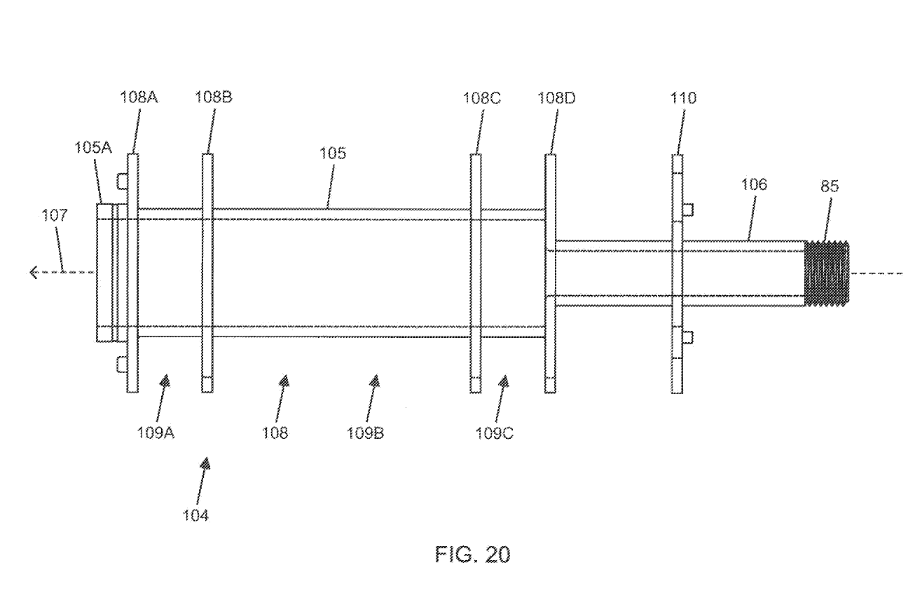

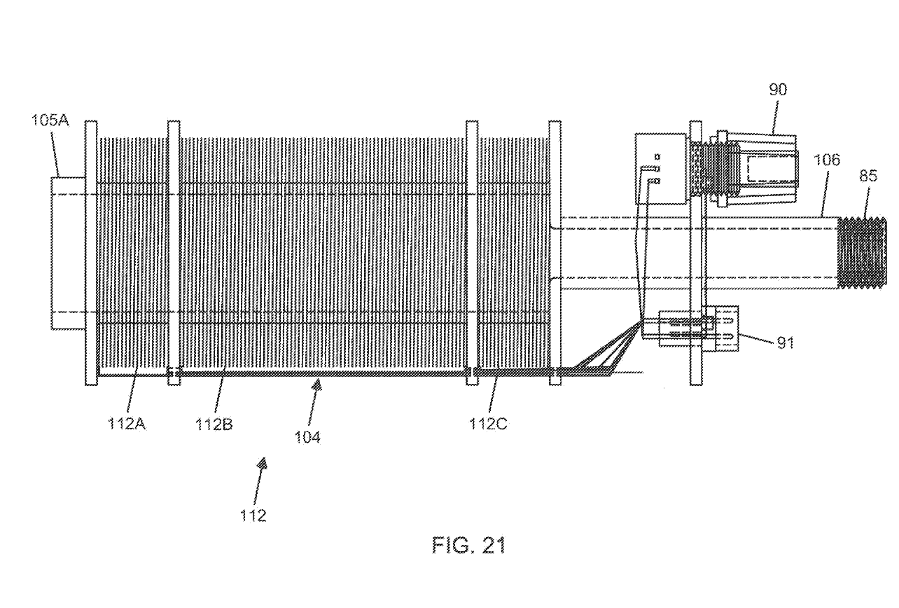

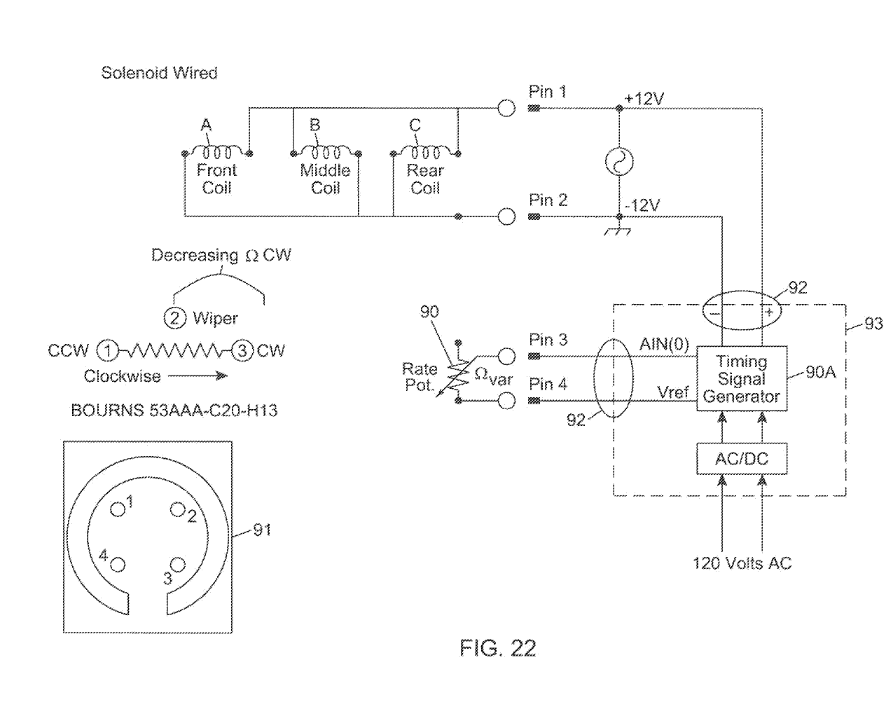

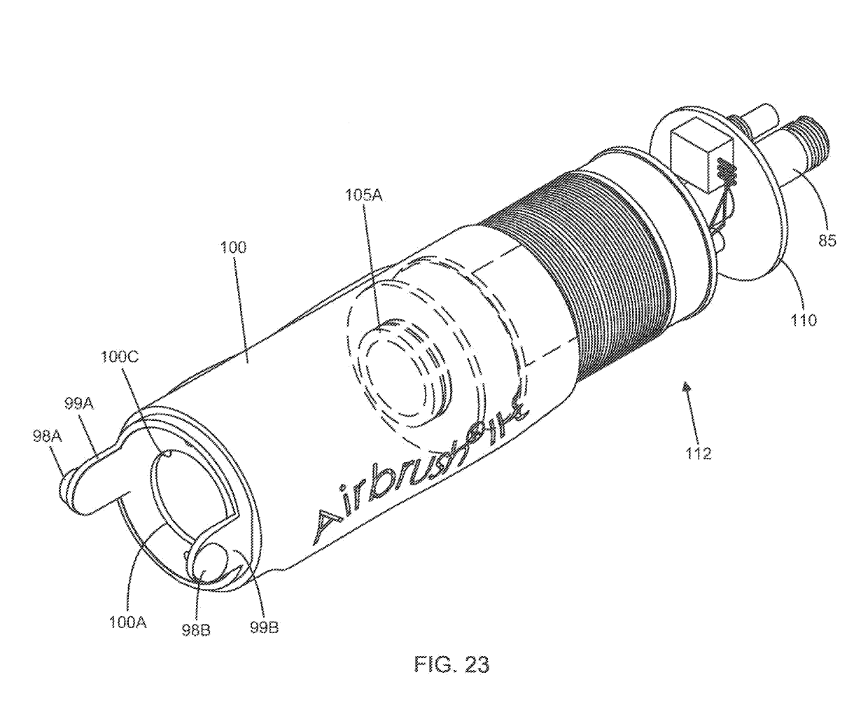

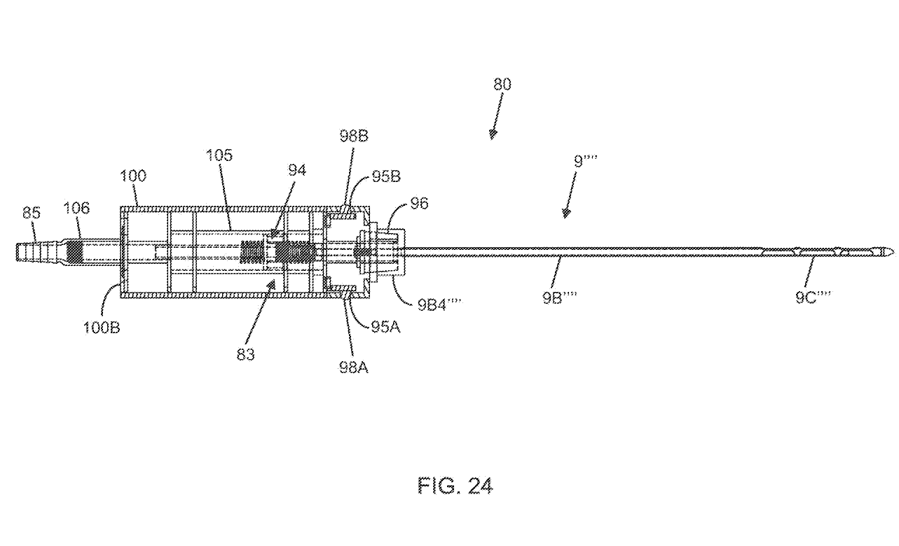

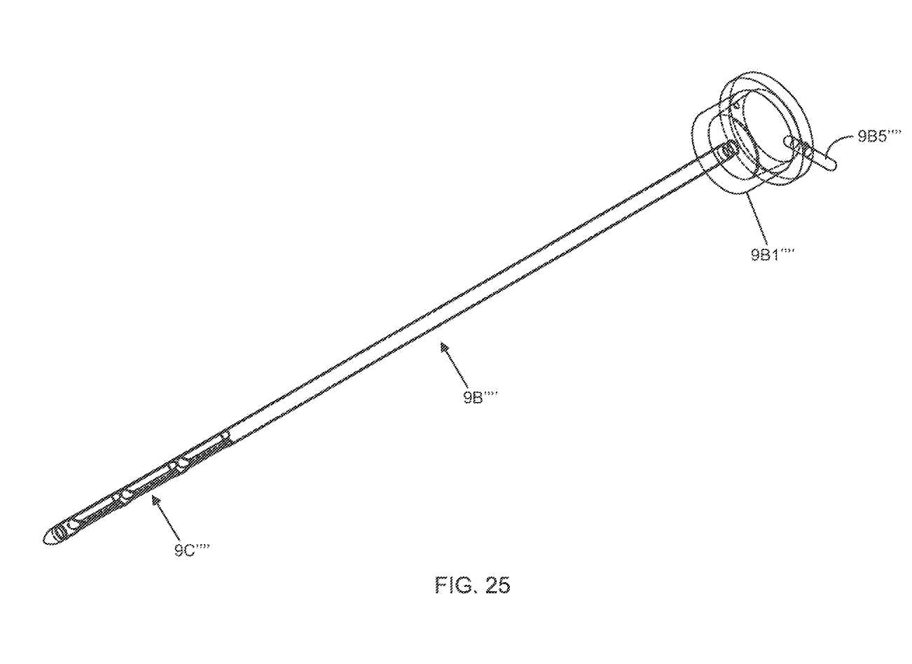

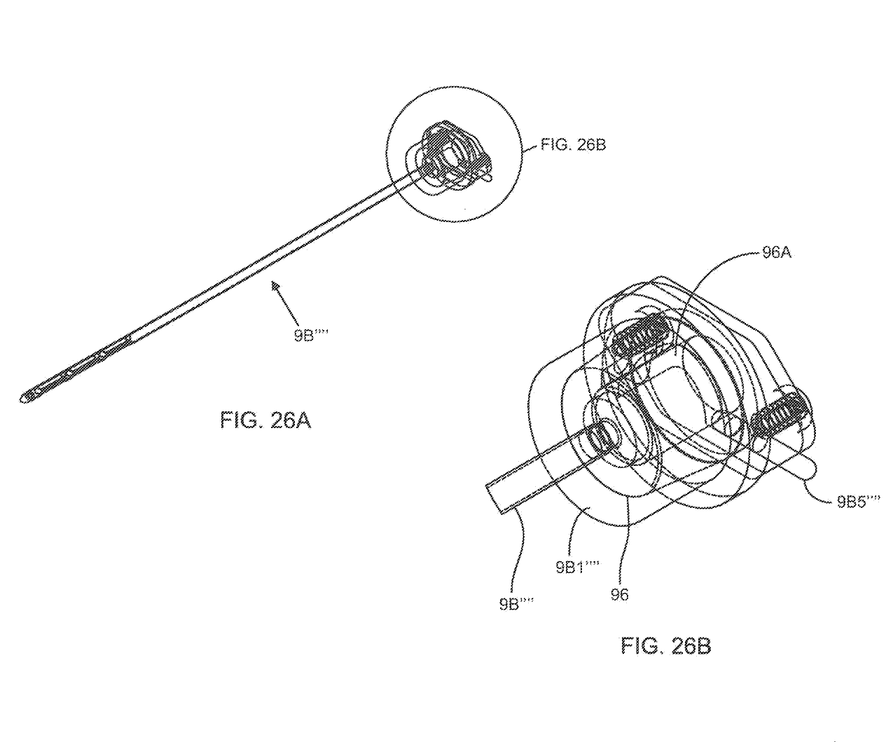

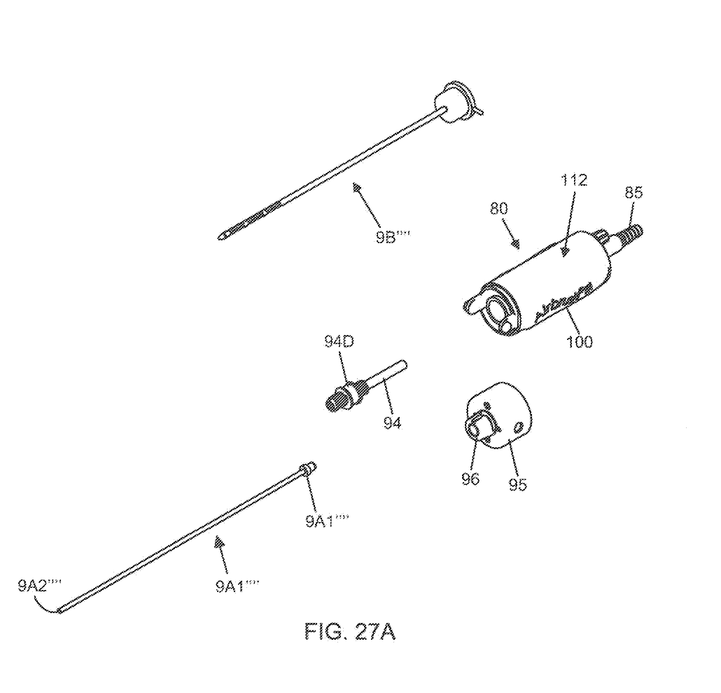

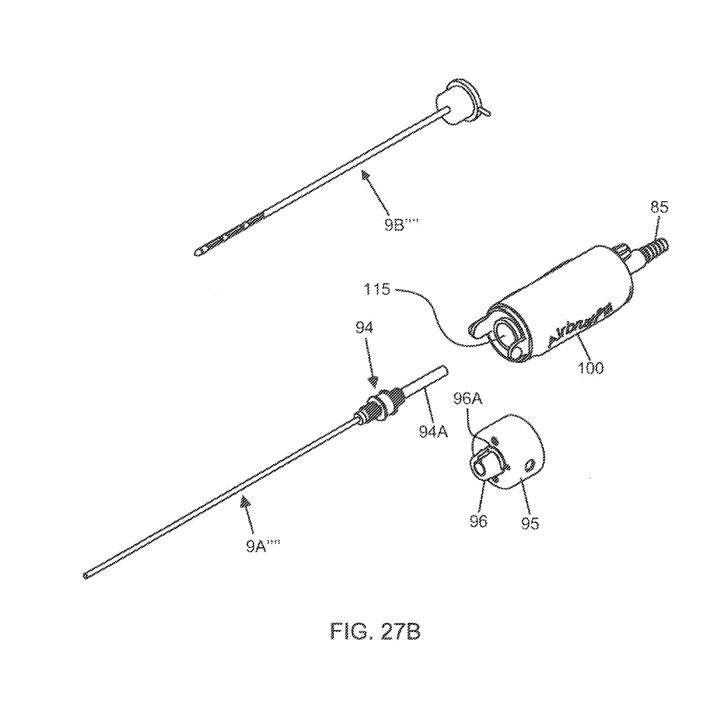

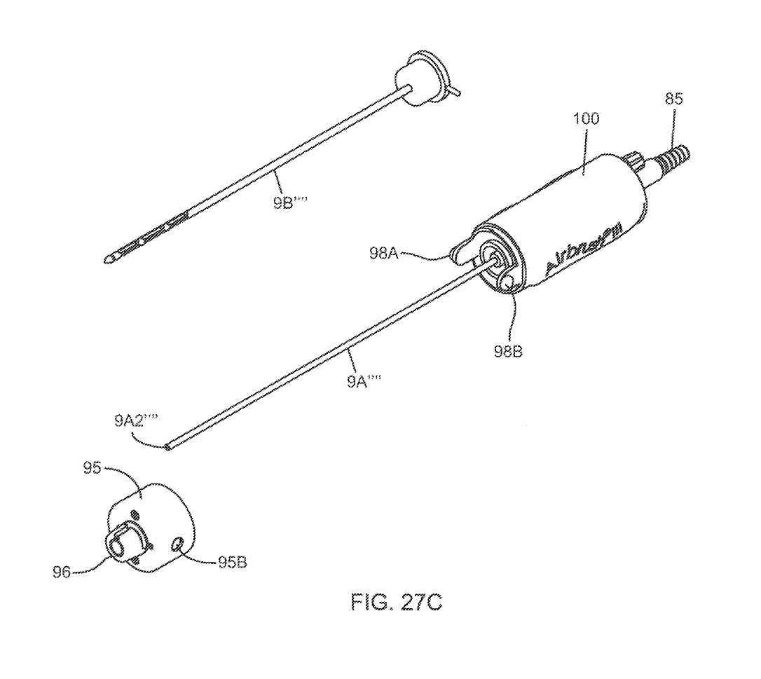

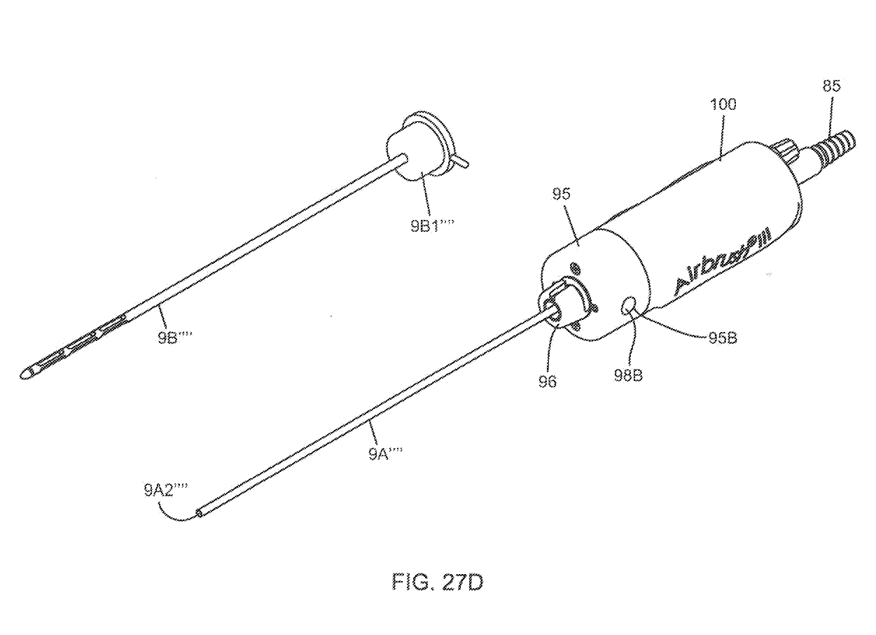

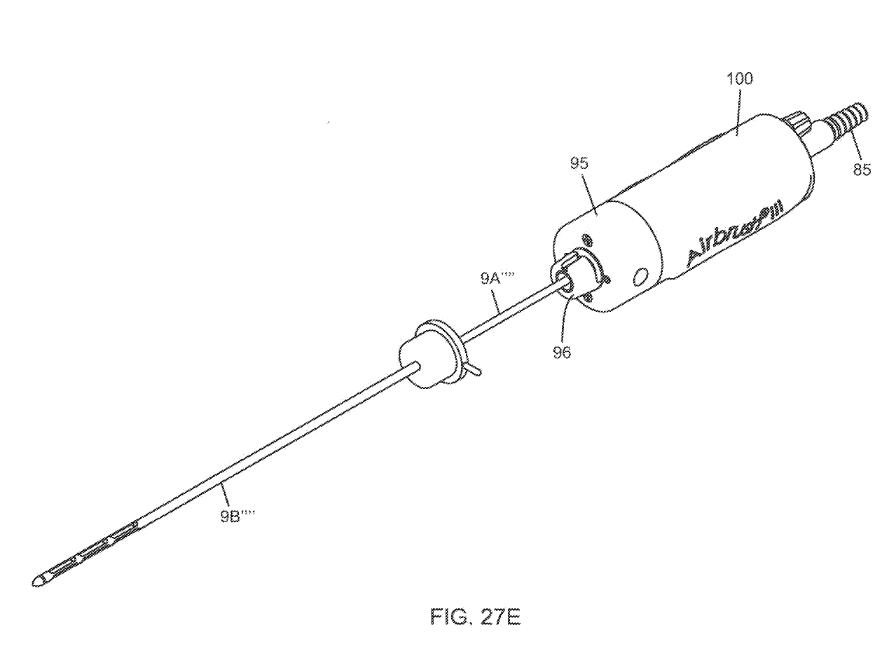

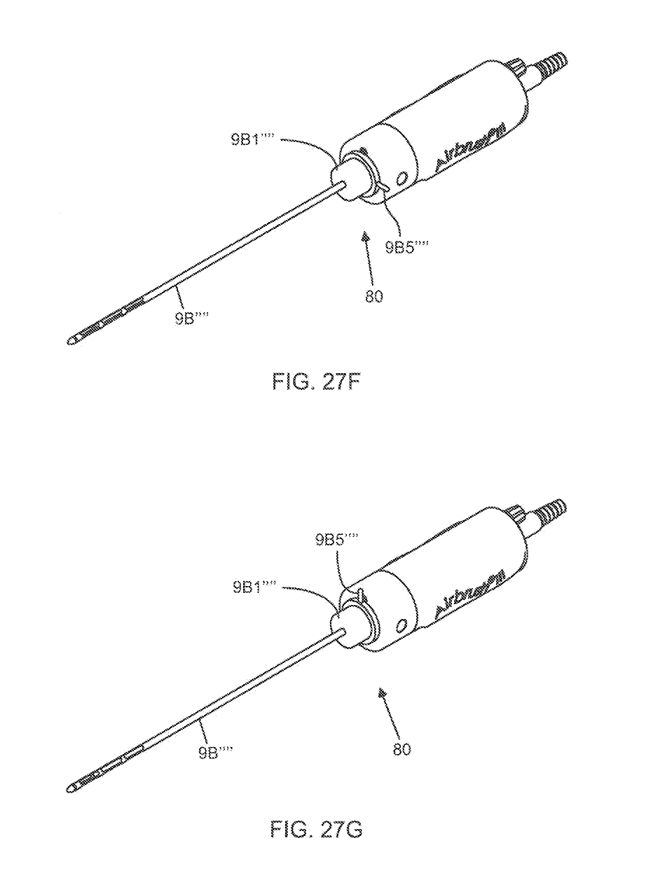

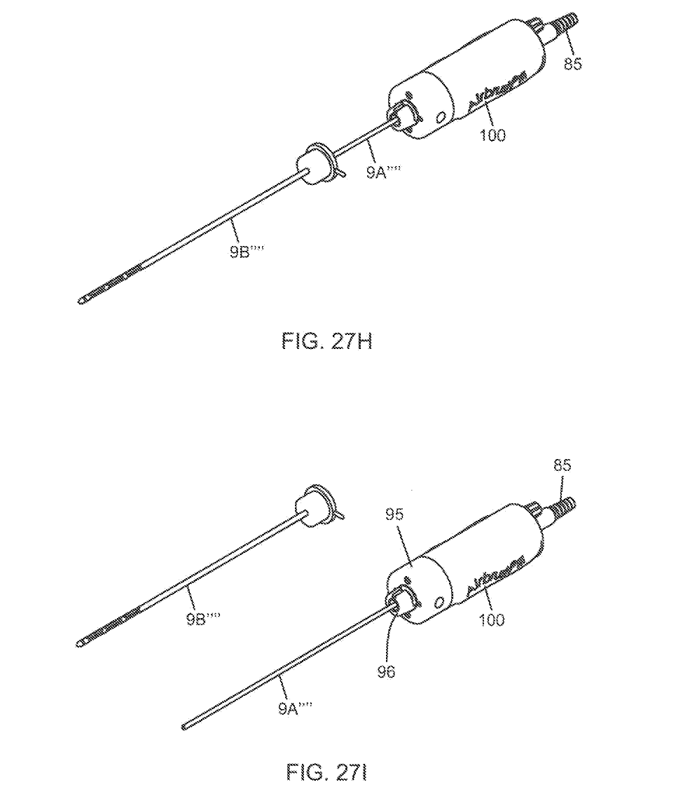

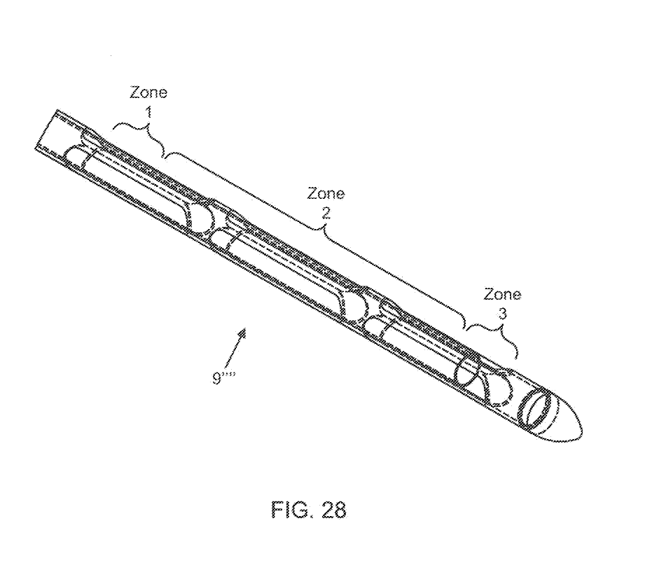

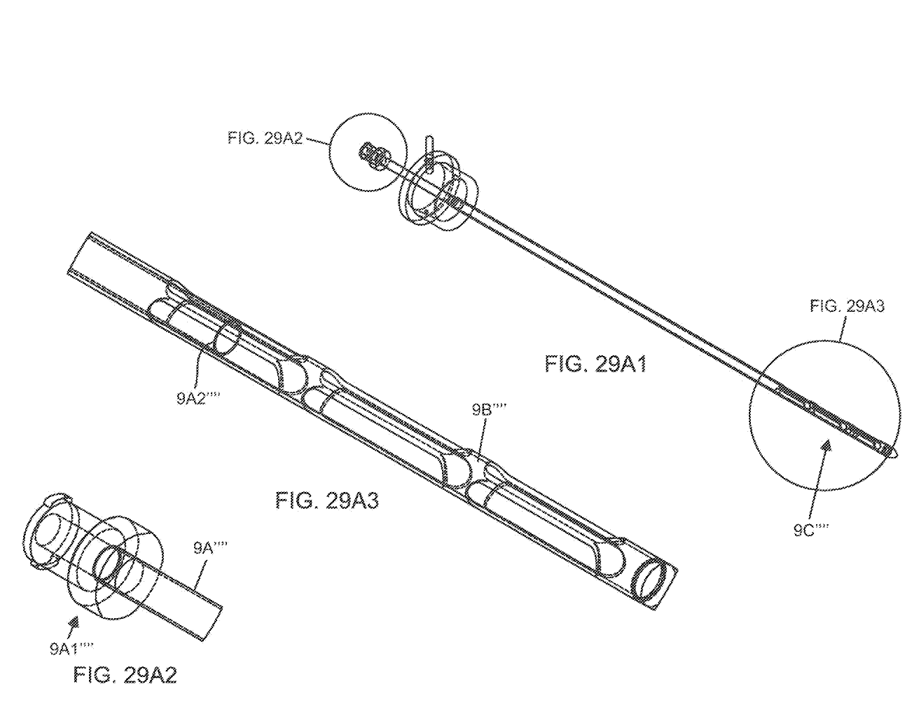

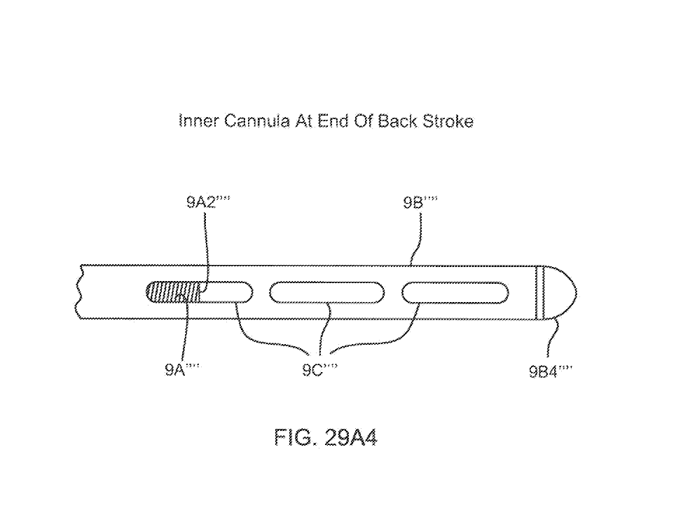

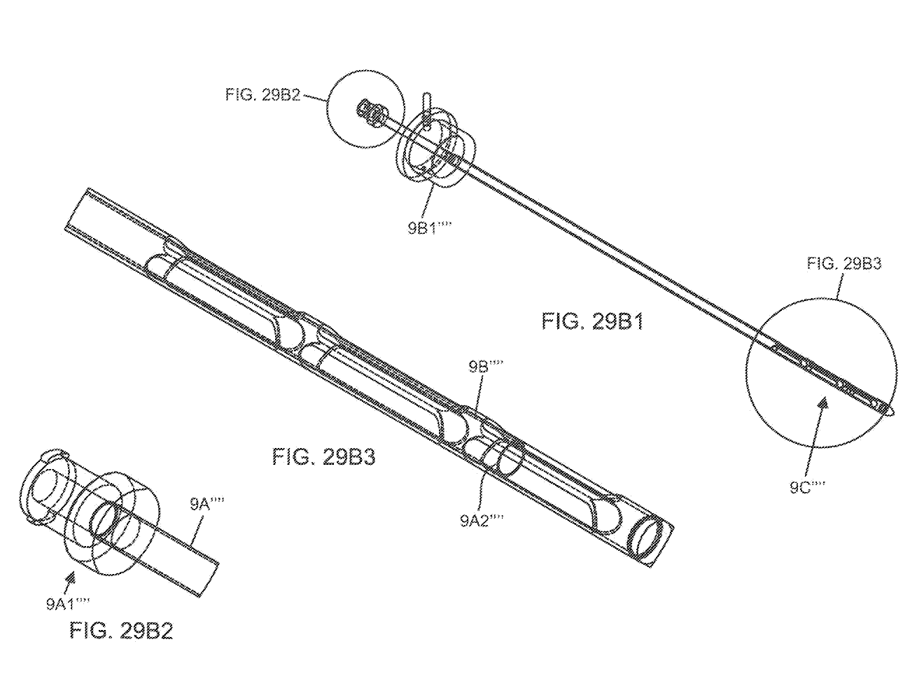

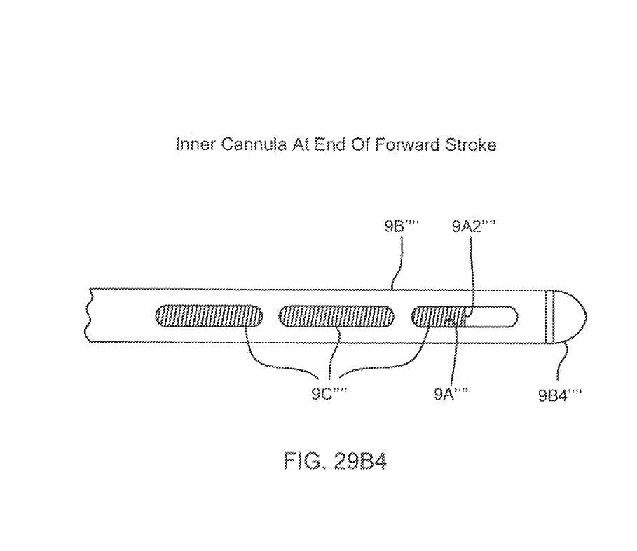

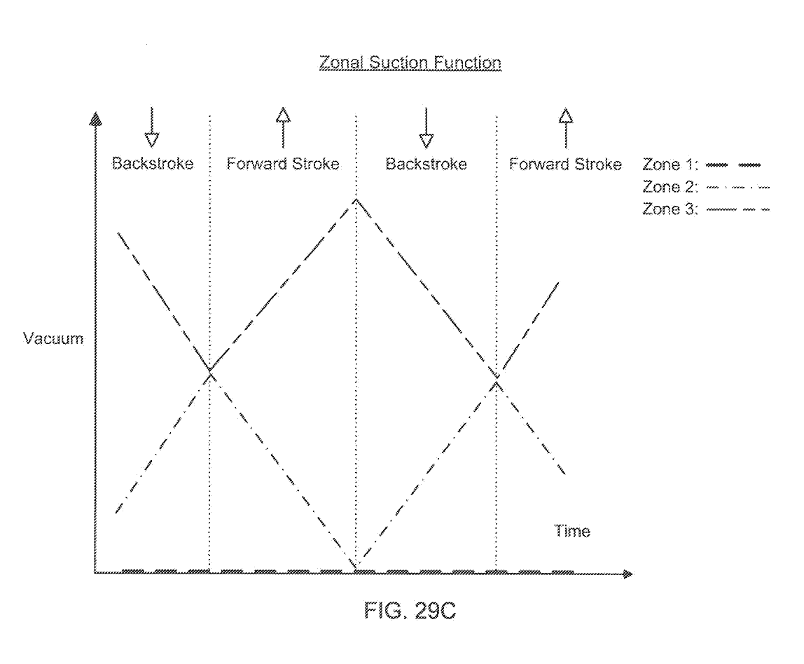

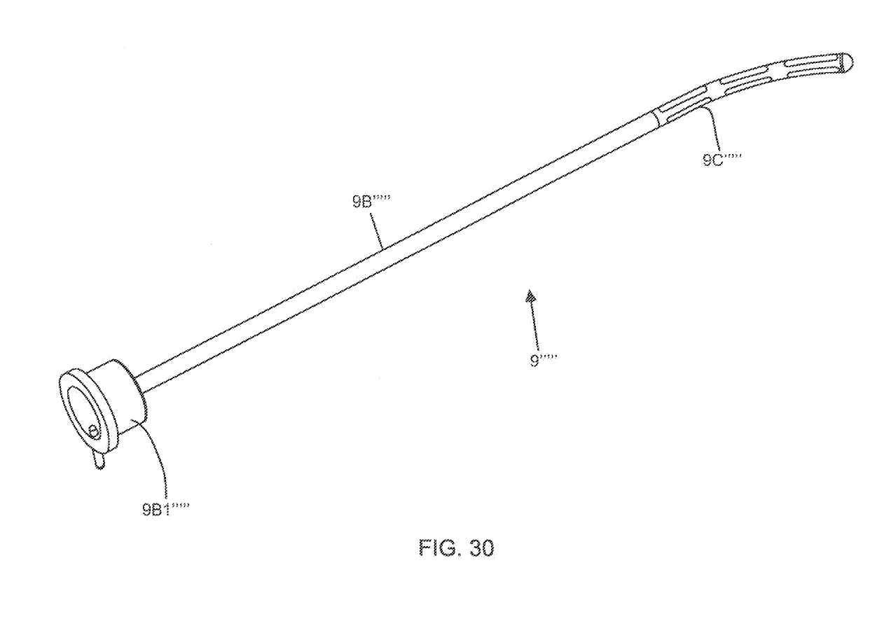

FIG. 3B is a perspective view of the outer cannula designed installation over the inner cannula shown in FIG. 3, and releasable attached to the front portion of the hand-supportable housing;

FIG. 4A is a first exploded view of the hand-supportable tissue aspiration instrument of FIGS. 2A and 2B, showing its primary components arranged in a disassembled state;

FIG. 4B is a second exploded view of the hand-supportable tissue aspiration instrument of FIGS. 2A and 2B, showing a first step in a multi-step assembly process used to construct the hand-supportable tissue aspiration instrument of the present invention;

FIG. 4C is a third exploded view of the hand-supportable tissue aspiration instrument of FIGS. 2A and 2B, showing a second step in a multi-step assembly process used to construct the hand-supportable tissue aspiration instrument of the present invention;

FIG. 5A is a perspective view of the back housing plate, employed in the hand-supportable tissue aspiration instrument shown in FIG. 2;

FIG. 5B is a perspective view of the cylindrical guide tube supporting the first and second electromagnetic coils employed in the hand-supportable tissue aspiration instrument shown in FIG. 2;

FIG. 5C is an elevated side view of the cylindrical guide tube supporting the first and second electromagnetic coils, employed in the hand-supportable tissue aspiration instrument shown in FIG. 2;

FIG. 5D is a perspective partially-cutaway view showing the connection of the two electromagnetic coils to the contact plug employed in the hand-supportable tissue aspiration instrument of the present invention illustrated in FIG. 2;

FIG. 5E is schematic diagram of a two coil push-pull type of circuit for enabling the cannula drive mechanism employed in the hand-supportable tissue aspiration instrument shown in FIG. 2;

FIG. 6A is a perspective view of the fenestrated distal tip portion of the twin-cannula assembly of the present invention, indicating the location of its three primary zones of vacuum pressure along the distal portion thereof, namely ZONE 1, ZONE 2 and ZONE 3;

FIG. 6B1 is a perspective view of the twin-cannula assembly of a first illustrative embodiment shown removed from the hand-supportable tissue aspiration instrument shown in FIG. 2, for purposes of illustration;

FIG. 6B2 is a partially cut-away enlarged view of the distal portion of the twin-cannula assembly illustrated in FIG. 6B1, when its open-ended inner cannula is slidably disposed at an extreme backward most position within the fenestrated (i.e. apertured) outer cannula, terminated in a blunt, bullet-nose shaped distal tip portion;

FIG. 6B3 is an enlarged perspective view of the distal portion of the twin-cannula assembly shown in FIG. 6B1, when its open-ended inner cannula is slidably disposed at the end of the backstroke position within the fenestrated outer cannula;

FIG. 6C1 is a perspective view of the twin-cannula assembly of a first illustrative embodiment shown removed from the hand-supportable tissue aspiration instrument shown in FIG. 2, for purposes of illustration;

FIG. 6C2 is a partially cut-away enlarged view of the distal portion of the twin-cannula assembly illustrated in FIG. 6C1, when its open-ended inner cannula is slidably disposed at an extreme forward most position within the fenestrated (i.e. apertured) outer cannula, terminated in a blunt, bullet-nose shaped distal tip portion;

FIG. 6C3 is an enlarged perspective view of the distal portion of the twin-cannula assembly shown in FIG. 6C1, when its open-ended inner cannula is slidably disposed at the end of the forward stroke position within the fenestrated outer cannula;

FIG. 6D is a vacuum pressure versus time graph illustrating the vacuum strength over the three primary zones along the twin-cannula assembly of FIGS. 6A through 6C3, during a complete inner-cannula reciprocation cycle, providing a zonal suction function specifying the performance of the fat tissue aspiration instrument used with the twin-cannula assembly;

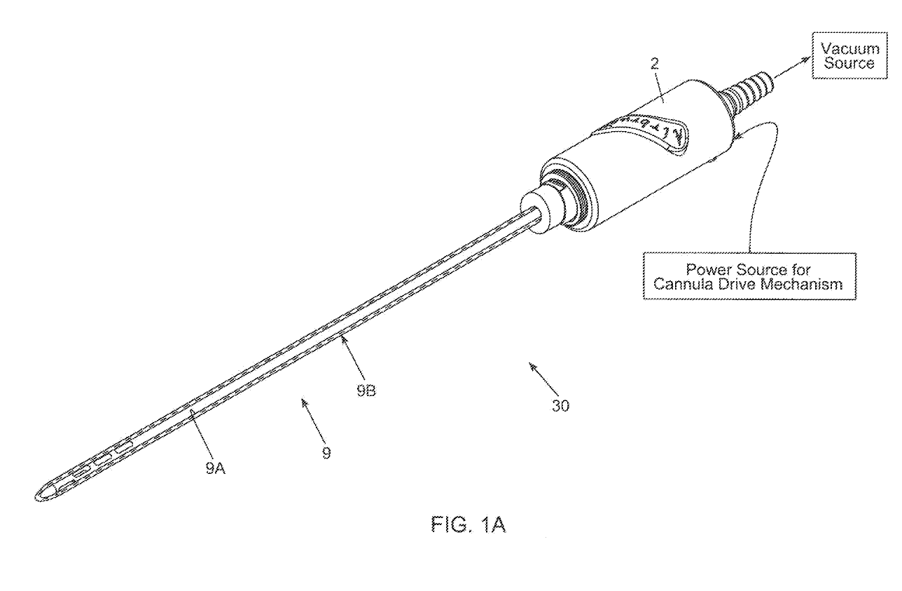

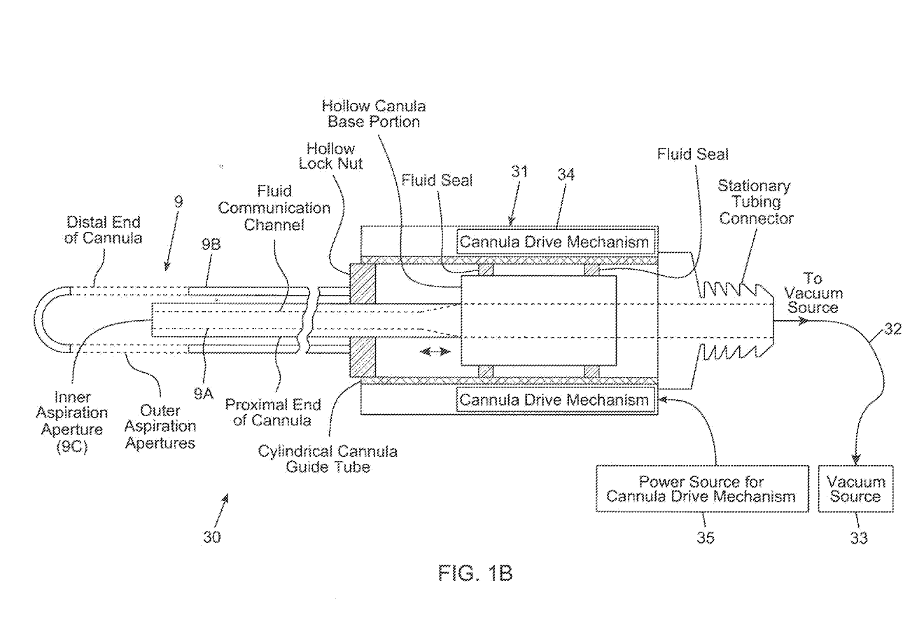

FIG. 7A is a perspective view of a second illustrative embodiment of the tissue aspiration instrumentation system of the present invention, modeled after the general design shown in FIGS. 1A and 1B, and shown comprising a hand-supportable tissue aspiration instrument having (i) a hand-supportable housing with a stationary tubing connector provided at the rear of the housing and receiving a length of flexible tubing connected to a vacuum source, and (ii) a twin-cannula RF-based bipolar electro-cauterizing assembly having an inner cannula coupled to a pneumatically-powered cannula drive mechanism disposed within the hand-supportable housing and powered by a source of pressurized air or other gas, while its fenestrated outer cannula is releasably connected to the front portion of the hand-supportable housing;

FIG. 7B is an elevated side view of the air-powered tissue aspiration instrument shown in FIG. 7A, wherein a single-button quick connect plug and associated multi-core cable assembly is provided on the rear portion of the hand-supportable housing, for supporting two gas lines and three electric wires between the instrument and its controller in a single bundle;

FIG. 7C is a partially exploded diagram of the second illustrative embodiment of the tissue aspiration instrumentation system of the present invention, showing its hand-supporting housing, in which its cylindrical (cannula base portion) guide tube and air-powered driven mechanism are installed, while its cannula base portion, cannula and cannula lock nut are shown disassembled outside of the hand-supportable housing, and its outer cannula not shown for purposes of illustration;

FIG. 7D is a perspective view of the outer cannula assembly used in the second illustrative embodiment of the tissue aspiration instrumentation system shown in FIG. 7A;

FIG. 8A is a cross-sectional view of the hand-supportable tissue aspiration instrumentation system of FIG. 7A, shown configured with its aspiration source, its controller and pneumatic power source, and multi-core cable assembly;

FIG. 8B is a schematic representation of the controller (and air-power supply) console depicted in hybrid schematic diagram of FIG. 8A, illustrating the front and rear Hall-effect cannula base position sensors installed within the hand-supportable housing of the instrument, the LCD panel, communication ports, LED indicators, and panel membrane switches supported on the controller console housing, as well as the ADC, digital signal processor (DSP) and DAC and proportional valve contained within the controller console housing and supplying gas tubes (via the multi-code cable assembly), and a supply of pressurized gas supplied to the controller housing, for driving the cannula drive mechanism of this embodiment of the present invention;

FIG. 9A is an elevated side view of the base portion of the inner cannula component used in the bipolar electro-cauterizing cannula assembly for the tissue aspiration instrument shown in FIG. 7A;

FIG. 9B is a end view of the base portion of the inner cannula shown in FIG. 9A, showing how adjacent pairs of conductive wires embedded in the plastic inner cannula are supplied with bipolar RF power signals, when a two pole power plug is inserted into the side wall of the base portion;

FIG. 9C is a perspective view of the plastic inner cannula with embedded wire conductors for conducting RF power signals to the distal portion of the fenestrated outer cannula;

FIG. 10A is a perspective view of the fenestrated distal tip portion of the twin-cannula assembly shown in FIG. 7A, indicating the location of its three primary zones of vacuum pressure along the distal portion thereof, namely ZONE 1, ZONE 2 and ZONE 3;

FIG. 10B1 is a perspective view of the RF bipolar electro-cautery twin-cannula assembly of a second illustrative embodiment, shown removed from the hand-supportable tissue aspiration instrument shown in FIG. 7A, for purposes of illustration;

FIG. 10B2 is a partially cut-away enlarged view of the distal portion of the twin-cannula assembly illustrated in FIG. 10B1, when its open-ended inner cannula is slidably disposed at an extreme backward most position within the fenestrated (i.e. apertured) outer cannula, terminated in a blunt, bullet-nose shaped distal tip portion;

FIG. 10B3 is an enlarged perspective view of the distal portion of the twin-cannula assembly shown in FIG. 10B1, when its open-ended inner cannula is slidably disposed at the end of the backstroke position within the fenestrated outer cannula;

FIG. 10C1 is a perspective view of the twin-cannula assembly of a first illustrative embodiment shown removed from the hand-supportable tissue aspiration instrument shown in FIG. 7A, for purposes of illustration;

FIG. 10C2 is a partially cut-away enlarged view of the distal portion of the twin-cannula assembly illustrated in FIG. 10C1, when its open-ended inner cannula is slidably disposed at an extreme forward most position within the fenestrated (i.e. apertured) outer cannula, terminated in a blunt, bullet-nose shaped distal tip portion;

FIG. 10C3 is an enlarged perspective view of the distal portion of the twin-cannula assembly shown in FIG. 10C1, when its open-ended inner cannula is slidably disposed at the end of the forward stroke position within the fenestrated outer cannula;

FIG. 10D is a vacuum pressure versus time graph illustrating the vacuum strength over the three primary zones along the twin-cannula assembly of FIGS. 10A through 10C3, during a complete inner-cannula reciprocation cycle, providing a zonal suction function specifying the performance of the fat tissue aspiration instrument used with the twin-cannula assembly;

FIG. 11A is a perspective view of a third illustrative embodiment of the tissue aspiration instrumentation system of the present invention, comprising a hand-supportable tissue aspiration instrument having an interior payload (i.e. bay) compartment with a hinged door panel for loading the inner cannula through the bay and out the front opening in the housing, and then connecting the flexible vacuum tube to the barbed end connector thereof, so that a pneumatically-powered (or electromagnetically-powered) cannula drive mechanism within the housing, can then drive the RF bipolar electro-cauterizing inner cannula within a stationary outer cannula, releasibly mounted to front portion of the hand-supportable housing, while the instrument is controlled by a control console as generally described in FIG. 8B;

FIG. 11B is an enlarged perspective of the distal portion of the twin-cannula assembly connected to the air-powered tissue aspiration instrument shown in FIG. 11A;

FIG. 11C is a perspective view of disassembled inner and outer cannula components of the twin-cannula assembly used in the instrument shown in FIG. 11A;

FIG. 11D is an enlarged view of the distal portion of the outer cannula shown in FIG. 11C;

FIG. 12A is a perspective view of the fenestrated distal tip portion of the twin-cannula assembly shown in FIG. 11A, indicating the location of its three primary zones of vacuum pressure along the distal portion thereof, namely ZONE 1, ZONE 2 and ZONE 3;

FIG. 12B1 is a perspective view of the RF bipolar electro-cautery twin-cannula assembly of a second illustrative embodiment, shown removed from the hand-supportable tissue aspiration instrument shown in FIG. 11A, for purposes of illustration;

FIG. 12B2 is a partially cut-away enlarged view of the distal portion of the twin-cannula assembly illustrated in FIG. 12B1, when its open-ended inner cannula is slidably disposed at an extreme backward most position within the fenestrated (i.e. apertured) outer cannula, terminated in a blunt, bullet-nose shaped distal tip portion;

FIG. 12B3 is an enlarged perspective view of the distal portion of the twin-cannula assembly shown in FIG. 12B1, when its open-ended inner cannula is slidably disposed at the end of the backstroke position within the fenestrated outer cannula;

FIG. 12C1 is a perspective view of the twin-cannula assembly of a first illustrative embodiment shown removed from the hand-supportable tissue aspiration instrument shown in FIG. 11A, for purposes of illustration;

FIG. 12C2 is a partially cut-away enlarged view of the distal portion of the twin-cannula assembly illustrated in FIG. 12C1, when its open-ended inner cannula is slidably disposed at an extreme forward most position within the fenestrated (i.e. apertured) outer cannula, terminated in a blunt, bullet-nose shaped distal tip portion;

FIG. 12C3 is an enlarged perspective view of the distal portion of the twin-cannula assembly shown in FIG. 12C1, when its open-ended inner cannula is slidably disposed at the end of the forward stroke position within the fenestrated outer cannula;

FIG. 12D is a vacuum pressure versus time graph illustrating the vacuum strength over the three primary zones along the twin-cannula assembly in FIG. 11A, during a complete inner-cannula reciprocation cycle, providing a zonal suction function specifying the performance of the fat tissue aspiration instrument used with the twin-cannula assembly;

FIG. 13A is a perspective view of a fourth illustrative embodiment of the tissue aspiration instrumentation system of the present invention, comprising a hand-supportable tissue aspiration instrument having an interior payload (i.e. bay) compartment with a hinged door panel for loading the inner cannula through the bay and out the front opening in the housing, and then connecting the flexible vacuum tube to the barbed end connector thereof, so that a pneumatically-powered (or electromagnetically-powered) cannula drive mechanism within the housing, can then drive the RF bipolar electro-cauterizing inner cannula within a stationary outer cannula, releasably mounted to front portion of the hand-supportable housing;

FIG. 13B is an enlarged perspective of the distal portion of the twin-cannula assembly connected to the air-powered tissue aspiration instrument shown in FIG. 13A;

FIG. 13C is a perspective view of a disassembled inner and outer cannula components of the twin-cannula assembly used in the instrument shown in FIG. 13A;

FIG. 13D is an enlarged view of the distal portion of the outer cannula shown in FIG. 13C;

FIG. 14 is a perspective view of the disposable electro-cauterizing inner cannula carrying both sides of the bipolar electro-cauterizing circuitry employed in the system shown in FIG. 12A;

FIG. 14A is an elevated side view of the base portion of the inner cannula component used in the bipolar electro-cauterizing cannula assembly for the tissue aspiration instrument shown in FIG. 3A;

FIG. 14B is an end view of the base portion of the inner cannula shown in FIG. 14, showing how adjacent pairs of conductive wires embedded in the plastic inner cannula are supplied with bipolar RF power signals, when a two pole power plug is inserted into the side wall of the base portion;

FIG. 14C is a perspective view of the plastic inner cannula with embedded wire conductors for conducting RF power signals to the distal portion of the fenestrated outer cannula;

FIG. 15A is a perspective view of the fenestrated distal tip portion of the twin-cannula assembly shown in FIG. 13A, indicating the location of its three primary zones of vacuum pressure along the distal portion thereof, namely ZONE 1, ZONE 2 and ZONE 3;

FIG. 15B1 is a perspective view of the RF bipolar electro-cautery twin-cannula assembly of a fourth illustrative embodiment, shown removed from the hand-supportable tissue aspiration instrument shown in FIG. 13A, for purposes of illustration;

FIG. 15B2 is a partially cut-away enlarged view of the distal portion of the twin-cannula assembly illustrated in FIG. 15B1, when its open-ended inner cannula is slidably disposed at an extreme backward most position within the fenestrated (i.e. apertured) outer cannula, terminated in a blunt, bullet-nose shaped distal tip portion;

FIG. 15B3 is an enlarged perspective view of the distal portion of the twin-cannula assembly shown in FIG. 15B1, when its open-ended inner cannula is slidably disposed at the end of the backstroke position within the fenestrated outer cannula;

FIG. 15C1 is a perspective view of the twin-cannula assembly of a fourth illustrative embodiment shown removed from the hand-supportable tissue aspiration instrument shown in FIG. 13A, for purposes of illustration;

FIG. 15C2 is a partially cut-away enlarged view of the distal portion of the twin-cannula assembly illustrated in FIG. 15C1, when its open-ended inner cannula is slidably disposed at an extreme forward most position within the fenestrated (i.e. apertured) outer cannula, terminated in a blunt, bullet-nose shaped distal tip portion;

FIG. 15C3 is an enlarged perspective view of the distal portion of the twin-cannula assembly shown in FIG. 15C1, when its open-ended inner cannula is slidably disposed at the end of the forward stroke position within the fenestrated outer cannula;

FIG. 15D is a vacuum pressure versus time graph illustrating the vacuum strength over the three primary zones along the twin-cannula assembly in FIG. 13A, during a complete inner-cannula reciprocation cycle, providing a zonal suction function specifying the performance of the fat tissue aspiration instrument used with the twin-cannula assembly;

FIG. 16A is a perspective view of the fifth illustrative embodiment of the hand-supportable tissue aspiration instrument, employing an improved twin-cannula assembly of the present invention, wherein the open-ended type inner cannula is driven by an electromagnetic cannula driven mechanism contained with the hand-piece portion of the instrument;

FIG. 16B is a rear-end axial view of the fifth illustrative embodiment of the hand-supportable tissue aspiration instrument shown in FIG. 16A;

FIG. 16C is a perspective view of the assembled twin-cannula assembly employed in the instrument of FIGS. 16A and 16B, but removed and detached from its hand-piece;

FIG. 16D is a perspective view of the dissembled twin-cannula assembly employed in the instrument of FIGS. 16A and 16B, but removed and detached from its hand-piece;

FIG. 17A is a perspective view of the hand-supportable tissue aspiration instrument of FIGS. 16A through 16D, shown with the fenestrated outer cannula removed off the attached inner cannula, and the clip-on housing nose cover removed off the front-end of the hand-supportable housing;

FIG. 17B is a perspective view of the hand-supportable tissue aspiration instrument of FIGS. 16A through 16D, shown with the fenestrated outer cannula removed off the attached inner cannula, the clip-on housing nose cover removed off the front-end of the hand-supportable housing, and the inner cannula-with its coupled inner base portion, removed from the front end of the hand-supportable housing;

FIG. 17C is a perspective view of the hand-supportable tissue aspiration instrument of FIGS. 16A through 16D, shown with the fenestrated outer cannula removed off the attached inner cannula, the clip-on housing nose cover removed off the front-end of the hand-supportable housing, the inner cannula removed from the front end of the hand-supportable housing, and its inner base portion decoupled from the inner cannula;

FIG. 17D is a perspective exploded view of the inner cannula base portion showing its hollow base portion tube with a first fluid seal disposed about its mid-portion, and a permanent magnetic ring, a second fluid seal, and a pair of return springs;

FIG. 17E is a perspective view of the inner cannula used in the twin-cannula assembly on the instrument of FIGS. 16A and 16B;

FIG. 17F is an enlarged perspective view of the proximal end of the inner cannula, showing its end portion adapted to couple with its inner cannula base portion;

FIG. 18A is a plan exploded view of the inner cannula assembly of the tissue aspiration instrument shown in FIGS. 16A and 16B;

FIG. 18B is a plan view of the assembled inner cannula assembly employed on the tissue aspiration instrument shown in FIGS. 16A and 16B;

FIG. 19 is a perspective view of the hand-supportable housing, in which the electromagnetic coil assembly and stationary rear tube connector is slid, during assembly;

FIG. 20 is an elevated side view of the cannula guide tube, electromagnetic coil support and aspiration tubing connection structure, about which the electromagnetic coil-winding support structure is formed by four spaced-apart annular flanges extending traverse to the longitudinal axis of the cannula guide tube portion, and defining three annular regions about the cannula guide tube where electromagnetic coiling windings can be would during manufacture;

FIG. 21 is an elevated side view of the electromagnetic-coil based cannula drive mechanism constructed from the cannula guide tube, electromagnetic coil support and aspiration tubing connection structure shown in FIG. 20;

FIG. 22 is a schematic diagram for the electromagnetic coil drive circuit employed in the twin-cannula tissue aspiration instrument shown in FIGS. 16A through 16D and FIG. 21;

FIG. 23 is a perspective view of electromagnetic-coil based drive mechanism of FIG. 21, being slid into the rear end opening of the hand-supportable housing shown in FIG. 19;

FIG. 24 is an elevated cross-sectional view of the twin-cannula tissue aspiration instrument shown in FIG. 16A, showing its magnet-bearing hollow inner cannula base portion slidably mounted within the guide tube surrounded by the electromagnetic coil structure;

FIG. 25 is a perspective view of the outer cannula used in connection with the twin-cannula tissue aspiration instrument shown in FIG. 24;

FIG. 26A is a perspective view of the outer cannula of FIG. 25 shown installed and locked to the cannula lock ring mounted on the clip-on housing nose cover installed on the hand-piece shown in FIG. 23;

FIG. 26B is an enlarged view of the base portion of the outer cannula installed on and locked to the cannula lock ring shown in FIG. 23;

FIG. 27A is an exploded view showing the disassembled primary components of the twin-cannula tissue aspiration instrument shown in FIG. 16A;

FIG. 27B is an exploded view showing the partial assembly of the primary components of the twin-cannula tissue aspiration instrument of FIG. 16A, where the inner cannula is coupled to its inner cannula base portion;

FIG. 27C is a perspective view showing the partial assembly of the primary components of the twin-cannula tissue aspiration instrument of FIG. 16A, showing the coupled inner cannula and its inner cannula base portion installed within the hand-piece component of the instrument;

FIG. 27D is a perspective view showing the partial assembly of the primary components of the twin-cannula tissue aspiration instrument of FIG. 16A, showing the coupled inner cannula and its inner cannula base portion installed within the hand-piece component of the instrument, and its clip-on housing nose cover clipped-on to the hand-supportable housing;

FIG. 27E is a perspective view showing the partial assembly of the primary components of the twin-cannula tissue aspiration instrument of FIG. 16A, showing the coupled inner cannula and its inner cannula base portion installed within the hand-piece component of the instrument, its clip-on housing nose cover clipped-on to the hand-supportable housing, and the outer cannula is being slid over the installed inner cannula;

FIG. 27F is a perspective view showing the partial assembly of the primary components of the twin-cannula tissue aspiration instrument of FIG. 16A, showing the coupled inner cannula and its inner cannula base portion installed within the hand-piece component of the instrument, its clip-on housing nose cover clipped-on to the hand-supportable housing, and the outer cannula installed over the installed inner cannula and rotated into its locked position;

FIG. 27G is a perspective view showing the partial assembly of the primary components of the twin-cannula tissue aspiration instrument of FIG. 16A, showing the coupled inner cannula and its inner cannula base portion installed within the hand-piece component of the instrument, its clip-on housing nose cover clipped-on to the hand-supportable housing, and the outer cannula installed over the installed inner cannula and rotated into its un-locked position;

FIG. 27H is a perspective view showing the partial disassembly of the twin-cannula tissue aspiration instrument of FIG. 16A, showing the coupled inner cannula and its inner cannula base portion installed within the hand-piece component of the instrument, its clip-on housing nose cover clipped-on to the hand-supportable housing, and its outer cannula being slid off the installed inner cannula;

FIG. 27I is a perspective view showing the partial disassembly of the twin-cannula tissue aspiration instrument of FIG. 16A, showing the coupled inner cannula and its inner cannula base portion installed within the hand-piece component of the instrument, its clip-on housing nose cover clipped-on to the hand-supportable housing, and its outer cannula being slid off the installed inner cannula;

FIG. 28 is a perspective view of the fenestrated distal tip portion of the twin-cannula assembly shown in FIG. 16A, indicating the location of its three primary zones of vacuum pressure along the distal portion thereof, namely ZONE 1, ZONE 2 and ZONE 3;

FIG. 29A1 is a perspective view of the twin-cannula assembly of the fifth illustrative embodiment, shown removed from the hand-supportable tissue aspiration instrument shown in FIG. 16A, for purposes of illustration, and configured when its open-ended inner cannula is slidably disposed at an extreme backward most position within the fenestrated outer cannula;

FIG. 29A2 is a partially cut-away enlarged view of the distal portion of the open-ended inner cannula shown in FIG. 29A2;

FIG. 29A3 is an enlarged perspective view of the distal portion of the twin-cannula assembly shown in FIG. 29A1, when its open-ended inner cannula is slidably disposed at the end of the backstroke position within the fenestrated outer cannula;

FIG. 29A4 is a partially cut-away enlarged view of the distal portion of the twin-cannula assembly illustrated in FIG. 29A1, when its open-ended inner cannula is slidably disposed at an extreme backward most position within the fenestrated outer cannula;

FIG. 29B1 is a perspective view of the twin-cannula assembly of the fifth illustrative embodiment, shown removed from the hand-supportable tissue aspiration instrument shown in FIG. 16A, for purposes of illustration, and configured when its open-ended inner cannula is slidably disposed at the end of the forward stroke position within the fenestrated outer cannula;

FIG. 29B2 is a partially cut-away enlarged view of the distal portion of the open-ended inner cannula shown in FIG. 29B2;

FIG. 29B3 is an enlarged perspective view of the distal portion of the twin-cannula assembly shown in FIG. 29B1, when its open-ended inner cannula is slidably disposed at the end of the forward stroke position within the fenestrated outer cannula;

FIG. 29B4 is a partially cut-away enlarged view of the distal portion of the twin-cannula assembly illustrated in FIG. 29B1, when its open-ended inner cannula is slidably disposed at an extreme backward most position within the fenestrated outer cannula, terminated in a blunt, bullet-nose shaped distal tip portion;

FIG. 29C is a vacuum pressure versus time graph illustrating the vacuum strength over the three primary zones along the twin-cannula assembly in FIG. 16A, during a complete inner-cannula reciprocation cycle, providing a zonal suction function specifying the performance of the fat tissue aspiration instrument used with the twin-cannula assembly; and

FIG. 30 is a curved outer cannula component for use with any of the tissue aspiration instruments of the illustrative embodiments employing a flexible plastic inner cannula, in accordance with the principles of the present invention.

### DETAILED DESCRIPTION OF THE ILLUSTRATED EMBODIMENTS

Referring to the figures in the accompanying Drawings, the various illustrative embodiments of the present invention will be described in great detail, wherein like elements will be indicated using like reference numerals.

Generalized Embodiment of the Tissue Aspiration Instrumentation System of the Present Invention, Provided with a New and Improved Twin Cannula Assembly

Referring to FIGS. 1A and 1B, a generalized embodiment of the tissue aspiration instrument of the present invention 30 will be described. As illustrated in FIGS. 1A and 1B, the tissue-aspiration instrument 30 comprises: a hand-supportable housing 31 adapted for receiving a length of flexible tubing 32 connected to a vacuum source 33, and a new and improved twin-cannula assembly 9 having an open-ended inner cannula 9A operably connected to the flexible vacuum tubing 32, and operably coupled to a cannula drive mechanism 34 that is disposed within the hand-supportable housing 31 and powered by an external power source (e.g. electrical power signals, pressurized air-streams, etc) 35, for reciprocating the open-ended inner cannula 9A within a stationary outer cannula 9B, having a fenestrated distal portion, and being releasably connected to the front portion of the hand-supportable housing.

In general, the base portion of the open-ended inner cannula 9A can be connected to the cannula drive mechanism 34 either internal to the hand-supportable housing 31, or external to the front end of the hand-supportable housing depending on the particular embodiment of the system. Also, the cannula drive mechanism 34 can be electromagnetically or pneumatically powered, to exert forces on the cannula base portion along the longitudinal axis of the cannula assembly (i.e. coaxially exerted on the cannula base portion) and cause the open-ended inner cannula 9A to reciprocate within the fenestrated outer cannula 9B, stationarily connected to the front portion of the hand-supportable housing 31, while fat adipose tissue is being aspirated along the outer aspiration apertures in the stationary outer cannula 9B, through the open-end of the inner cannula 9A, down the lumen of the reciprocating inner cannula 9A, and ultimately along the flexible tubing 32 towards the vacuum source 33.

When the cannula drive mechanism 34 is electromagnetically driven, it can be constructed from two or more spaced-apart electromagnetic wire coils wound about the cylindrical guide tube installed within the hand-supportable housing, and electrically connected to an electrical signal source. This will generate an electromagnetic force field which periodically pushes and pulls, for example, a permanent magnet ring coupled to an inner cannula base portion (connected to the inner cannula) and thereby causing (i) the hollow inner cannula base portion to reciprocate within a cylindrical guide tube, (ii) the hollow open-ended inner cannula to reciprocate within the stationary hollow outer cannula, and (iii) open-ended aspiration aperture 9A2 at the distal portion of the inner cannula 9B, to reciprocate along the elongated outer aspiration apertures of the stationary outer cannula 9B.

When the cannula drive mechanism is pneumatically driven, it can be constructed using an pneumatically source of pressurized air or gas, controllably supplied to a coaxially-arranged pneumatically-powered cannula drive mechanism, or linear actuator powered cannula drive mechanism.

In yet other embodiments, these elements may be realized in different ways without departing from the scope and spirit of the present invention.

First Illustrative Embodiment of the Tissue Aspiration Instrumentation System of the Present Invention, Provided with a New and Improved Twin Cannula Assembly

In FIGS. 2 through 6D, the first illustrative embodiment of power-assisted tissue-aspiration instrument system 30 is realized as a hand-supportable tissue aspiration instrument 40 comprising: a hand-supportable housing 2 having (i) a front portion 41 and a rear portion 42 aligned along a longitudinal axis; (ii) an interior volume 43 and a cylindrical guide tube 1 mounted within the interior volume of the hand-supportable housing 2; (iii) a cannula drive mechanism 44 disposed adjacent the cylindrical guide tube 1; and (iv) a stationary tubing connector 3 coaxially mounted to the rear portion of the hand-supportable housing along the longitudinal axis, connected to the cylindrical guide tube, and having an exterior connector portion permitting a section of flexible aspiration tubing 45 to be connected at its first end to the exterior connector portion 4, and where the second end of the section of flexible tubing 45 is connected to a vacuum source 46. The improved twin cannula assembly 9 comprises: a hollow outer cannula 9B with a fenestrated distal portion (i.e. having a plurality of outer aspiration apertures 9B3), a lumen portion 9B2, and an outer cannula base portion 9B1 stationarily connected to the front portion of the hand-supportable housing 2; and a hollow inner cannula 9A with an open-end aperture 9A3 and disposed within the hollow outer cannula 9B and having a leur-locking coupling 15 that connects with a leur-locking coupling 16 on a hollow inner cannula base portion 13.

As shown in FIG. 3A, the (disposable) cannula base portion 13 carries a permanent magnetic ring 8 removably attached to an actuator which slidably supports the cannula base portion 13 within the cylindrical guide tube 1. A seal is created by the tight fit between the tubular portion of the actuator and the surrounding stationary tube and barb annular extrusion behind the front magnet-fastening portion. This tube-within-a-tube feature behind the actuator allows a stationary barb 4. This tube-within-a-tube structure may be perfectly round (i.e. cylindrical), or ovoid or other geometry so as to maintain a fixed alignment of (i) actuator and inner cannula 9A, and (ii) outer cannula 9B. This tube-within-a-tube overlap needs to be equal to, or greater than, the stroke (i.e. the total to-fro motion) of the actuator within the solenoid assembly. Such an overlap with 0.002″ to 0.005″ tolerance between the inner and outer diameter surfaces of the hollow cannula base portion 13 within guide tube 1 should be adequate to eliminate vacuum leak without need of seals.

As shown, the cannula 9 is coupled to the cannula base portion 13 by way of a mated leur-lock coupling 15, 16, and the lumen extending within the cannula and its base portion is in fluid communication with the stationary tubing connector 3, by way of the interior volume of the cylindrical guide tube 1 extending between the cannula base portion 13 and the stationary tubing connector 4. The stationary tubing connector 3 (having a barbed tubing connector portion) is adapted to unscrew from the rear portion of the hand-supportable housing so that housing back plate 3 can be removed so that the cylindrical guide tube (i.e. the wound bobbin) can be slid into the hand-supportable housing 2. The top and bottom of the hollow cylindrical ring magnet 8 produce opposing magnetic poles, and magnet 8 is secured onto the cannula base portion 13 by way of nut 5 which screws onto a set of threads form on other surface of the cannula base portion. Alternatively two axially polarized ring magnets may be placed with same poles in contact on the actuator to augment the flux of the adjacent poles.

In the illustrative embodiment, the fluid seals 6, 7 are realized as a pair of thin-walled, collapsible (i.e. invertible) bell-shaped silicone sealing washers which act as front and rear diaphragms allowing motion of the cannula base portion 13 the cylindrical guide tube 1. By setting mid-point geometry, a single spring or spring-like diaphragm washer can effect a return stroke without need of coil polarity reversal, so that simple pulsing action will suffice. Front and rear coil windings 11 and 12 are formed about the outer surface of the cylindrical guide tube 1, and electrically connected to the connector plug 14 formed on the rear end of the hand-supportable housing 2.

FIG. 3B shows the outer cannula 9B installed over the inner cannula 9A, and connected to the front portion of the housing of the tissue aspiration instrument system 40. As shown, outer cannula 9B comprises: a base portion 9B1 with internal threads that screw over matching threads on the front portion of the hand-supportable housing 2; a lumen portion 9B2 extending from the base portion 9B1; and a fenestrated distal portion having multiple sets of aspiration apertures 9B2 and terminated with a blunt bullet-tip nose, as shown. Preferably, the outer cannula component 9B is made from a stainless steel, or other suitable material, as will be described in greater detail hereinbelow. However, optionally, it can be made from a disposable plastic material, depending on economics.

FIG. 4A shows a fully exploded view of the hand-supportable tissue aspiration instrument of FIGS. 2A and 2B, clearly revealing its dissembly of components, as comprising: cylindrical guide tube 1 with flanges for containing electromagnetic coil windings 11, 12, a hand-supportable housing 2, housing back plate 3, stationary tubing connector 4 with a vacuum tubing barb, a magnet fastening nut 5, a front washer 6, a back washer 7, a ring magnet 8, a cannula 9 provided with a leur-lock fastener 15, a front chamber screw cap 10, a back electromagnetic coil 11, a front electromagnetic coil 12, a disposable cannula base portion 13 realized as leur-lock fastener, a contact/connector plug 14 (e.g. Binder 719), a (male) leur-lock fitting 15, and a (female) leur-lock fitting 16.

FIGS. 4B and 4C show how the components in FIG. 4A can be assembled in a preferred manner during manufacture on an assembly line. After the hand-held instrument is fully assembled, the surgeon simply connects the inner cannula assembly 9A to the installed (disposable) cannula base portion 13, using a leur-lock coupling mechanism 15, 16, and then installs the fenestrated outer cannula 9B over the inner cannula 9A and couples it to the front end of the hand-supportable housing 2, to complete the assembly the instrument and prepare it for use in surgery.

Taken together, FIGS. 5A, 5B 5C and 5D show how the first and second electromagnetic coils 11, 12 are wound about the cylindrical guide tube 1, and then how wiring of these coils are electrically connected to the electrical connector mounted on the housing back plate 3, employed in the first illustrative embodiment shown in FIGS. 2A through 5E. FIG. 5E shows the schematic diagram depicting how the two coil 11 and 12 are driven by a push-pull type of circuit, for the purpose of enabling the cannula drive mechanism employed in the hand-supportable tissue aspiration instrument of the present invention illustrated in FIG. 3B.

Alternatively two smaller coils may be positioned at both poles of the central solenoid and reverse-wired so as to augment the magnetic flux at the ends of the longer central solenoid. Alternatively as well a ferrous or magnetically highly permeable material such as MuMetal may be used beneath the solenoid windings, or on top of the solenoid windings, to further augment the magnetic flux of the central and end solenoids. This may also serve to minimize magnetic flux and shield EMF external to the hand-supportable housing 2.

### Specification of the Improved Power-Assisted Twin-Cannula Assembly of the Present Invention

FIG. 6A shows the fenestrated distal tip portion of the twin-cannula assembly 9, indicating the location of its three primary zones of vacuum pressure along the distal portion thereof, namely ZONE 1, ZONE 2 and ZONE 3. FIG. 6B2 shows the distal portion of the twin-cannula assembly of FIG. 6B1, when its open-ended inner cannula 9A is slidably disposed at an extreme backward most position within the fenestrated (i.e. apertured) outer cannula 9B, terminated in a blunt, bullet-nose shaped distal tip portion 9B4. FIG. 6C2 shows the distal portion of the twin-cannula assembly of FIG. 6C1, when its open-ended inner cannula 9A is slidably disposed at an extreme backward most position within the fenestrated (i.e. apertured) outer cannula 9B.

As shown, the twin-cannula assembly 9 comprises: an outer cannula 9B mounted stationary to the front portion of a hand-supportable housing containing an inner cannula reciprocation mechanism; and an outer cannula 9B mounted over the inner cannula and stationary with respect to the hand-supportable housing. In the illustrative embodiments shown herein, the outer cannula 9B has three groups of outer aspiration apertures formed about its distal portion, namely: a first group of outer aspiration apertures closest to the proximal end of the outer cannula, designated as Zone 1; a third group of outer aspiration apertures closest to the distal end of the outer cannula designated as Zone 3; and a second group of outer aspiration apertures residing between the first and second groups of outer aspiration apertures, designated as Zone 2. The inner cannula 9A has an open-end type aspiration opening 9A2 that reciprocates back and forth to a mid position between the first group of aspiration apertures (Zone 1) and the third group of outer aspiration apertures (Zone 3), so that vacuum pressure is always delivered to at least ½ of one the outer aspiration aperture groups as the open-ended inner cannula 9A is reciprocated back and forward within the outer cannula 9B, cutting off fat being aspirated into the inner cannula lumen, and thereby progressively delivering more suction performance and achieving a scissoring-effect during tissue aspiration operations.

Notably, the improved twin-cannula tissue aspiration instrument of the present invention described above simultaneously solves multiple functional and production issues by modifying and improving the twin cannula design in significant ways. Specifically, as shown in FIGS. 6A through 6C2, multiple longitudinal slots (i.e. aspiration apertures) are circumferentially formed about the outer cannula 9B so that the outer cannula wall surface is heavily fenestrated, thereby (i) exposing a maximally large area of patient tissue to suction pressure within the interior of the outer cannula, while (ii) retaining sufficient structural support required to maintain the strength and structure of the outer cannula. At the same time, the inner cannula 9A has an open-ended aspiration aperture, which eliminates (i) costly steps relating to cutting holes, creating and welding bullet tips, and (ii) alignment issues as there are no holes to register within slots.

An alternative material to stainless steel for the inner cannula is nitinol (flexible nickel titanium alloy) as this “memory metal” allows the use of curve cannula embodiments. However, using a plastic inner cannula 9A allows an inexpensive angio-catheter style disposable plastic extrusion to replace an expensive metal part requiring very tight tolerancing. Using an open-ended inner cannula, as specified herein, allows very thin and inexpensive FEP plastics to be used with a very thin inner cannula 9A, supported by a rigid thicker outer cannula 9B, whether made of metal or plastic, thereby eliminating concerns about the inner diameter (ID) of the inner cannula when constructed from plastic. Also, the use of the open-ended inner cannula 9A eliminates alignment issues as there is no need to fix the axis of the inner cannula 9A. In turn, this allows simpler inner cannula mounts that may be front or back-loaded, without requiring an access door provide in the hand-hand instrument housing. The actuator, realized by the ring magnet and the inner cannula, may be conveniently provided in a single-use sterile peel-pack for use in a single surgery. In short, the novel twin-cannula design of the present invention allows inexpensive manufacturing, easier tolerancing, less expensive materials, and advantages in reduced size and complexity in cannula mounting.

In addition to the design and production advantages indicated above, the twin-cannula design of the present invention eliminates interval fat build-up and release problems that have reduced the applied-suction effectiveness of Applicant's prior art twin-cannula assemblies. In the twin-cannula design of the present invention, the length of the inner cannula 9A within the outer cannula 9B is specified so as to ensure: (1) that suction pressure is always applied to a minimal area of patient tissue (i.e. the suction passage is never completely occluded); (2) that suction pressure is applied to a very large area of patient tissue for the majority (e.g. ⅔rds) of the time; and (3) that a very high degree of suction pressure is applied to a smaller area of patient tissue, at the tip portion of the cannula, for about ⅓rd of the time. To achieve these objectives in the twin-cannula design shown in FIGS. 6A through 6C2, the length of the inner cannula 9A has been specified so that (i) it terminates its backstroke in the middle of the most proximal slots (i.e. outer aspiration apertures over Zone 1) as shown in FIGS. 6B1 through 6B3, and (ii) finishes its forward stroke in the middle of the most distal slots (i.e. outer aspiration apertures over Zone 3) as shown in FIGS. 6C1 through 6C3.

During system operation, twin-cannula assembly design of the present invention 9 employs a pulsatile vacuum pressure function which helps eliminate and “unclog” aspirated fat tissue build-ups along the suction path between the outer aspiration apertures and the vacuum pump, thereby providing smoother aspiration without the drawback of decreasing aspiration rates caused by reducing the cross section of aspirated tissue. A repetitive “pulsing” or “pulsatile” type suction action is achieved in the instrument of the present invention using a vacuum suction force (30-44 mm Hg) applied to areas of patient tissue around the circumference of the outer cannula 9B. This pulsing or “pulsatile” type suction action minimizes tissue accumulation and blockages and dislodging any build-ups or suction impediments with each and every cycle of inner cannula reciprocation. This pulsatile suction action serves to maintain a maximal sustained rate of suction pressure along the distal portion of the twin-cannula assembly 9, during fat tissue aspiration operations, while allowing increased aspiration efficiency and control.

The open-ended inner cannula 9A, and specially fenestrated outer cannula 9B, allows the twin-cannula assembly 9 to aspirate fat tissue aspiration during both forward and back stroke directions of the inner cannula 9A, without loss of suction pressure or the creation of fat plug build-up, characteristic of prior art twin-cannula assembly performance. As illustrated in FIGS. 6B1 through 6D, as the inner cannula 9A strokes down the outer cannula 9B, it cuts off or avulses stalks of fat tissue protruding through the multiple fenestrations (i.e. aspiration apertures 9B2) formed along the distal portion of the assembly.

When the inner cannula is advancing (“forward stroke”), the vacuum is augmented by the push of the cannula to lop off and push any aspirated tissue proximally (i.e. distal to proximal) down to the base of the inner cannula shaft. When the inner cannula is retracting (i.e. during the “backward stroke” of the inner cannula), no new tissue is likely to enter the open distal tip of the inner cannula. Suction will retain tissue that has already entered the inner cannula lumen, and still tend to move it down the shaft, but the back-stroke without presentation of new tissue at the open end of the inner cannula allows a momentary clearing of cannula contents. This backward stroke will also serve to avulse or “pluck off” aspirated tissue (e.g. globules of fat) from their vascular pedicles or stalks within the fibrous lattice of connective tissue surrounding fat cells. Vessels within these pedicles have been constricted by virtue of the dilute epinephrine (i.e. a potent vasoconstrictor) contained in tumescent solution, the saline or lactated ringer's solution used to tumescence, distend or “blow up” the area to be treated. This tumescent solution is generally combined with a local anesthetic (e.g. dilute xylocalne) to allow liposuction under local anesthesia and to minimize postoperative pain. This prepares the inner cannula for the next forward stroke.

With this improved design, an improved suction pressure distribution and the forward cannula motion combine to increase the speed and efficacy of tissue aspiration. Also, using an open-ended inner cannula 9A, as shown in FIGS. 6B3 and 6C3, the twin-cannula assembly 9 eliminates the possibility of the surgeon working against the instrument, as often occurs when using prior art twin-cannula assemblies.

The twin-cannula assembly 9 removes any obstructions along the suction path (i.e. from the vacuum pump to the distal tip of the cannula), and allows only a temporary build-up of aspirated tissue along the suction path. Consequently, open-ended inner cannula 9A in the twin-cannula assembly 9 is able to apply substantially uniform vacuum pressure, or a constant rate of suction pressure, to the cross sectional area of the one or more apertures of the outer cannula 9B, at every point in time, during its reciprocation cycle. Such improved vacuum pressure characteristics support an increased overall average rate of tissue aspiration. Notably, this is a comparatively small region of cross-sectional area, even with multiple apertures formed in the distal portion of the fenestrated outer cannula 9B.

Using the twin-cannula assembly 9, aspiration occurs in a very different fashion with a highly fenestrated outer cannula 9B and a grossly reciprocating open-ended inner cannula 9A. As shown in FIGS. 6A through 6C3, the outer cannula 9B is maximally fenestrated over an area which extends both proximal and distal to the inner cannula excursion. The limit to fenestration of the outer cannula 9B is the retention of structural integrity in the material, from which the outer cannula is made, metal or plastic, so that it avoids bending or breaking during tissue aspiration operations. As the tensile strength of metal is much higher than plastic, thinner-walled inner cannulas having grated-fenestrated cross-sectional areas, can be attained by working with No. 316 stainless steel (SS), as the preferred embodiment of outer cannula 9B.

As the inner cannula lumen 9A is open-ended, vacuum or suction pressure is applied to the cross sectional area of all the outer cannula fenestrations 9B2 which are distal to the open lumen of the inner cannula 9A. It is understood that a highly-fenestrated outer cannula 9B will aspirate tissue faster because (i) more apertures allow tissue to be sucked into the open-ended inner cannula 9A, and (ii) the “grated” surface serves as a tissue-disruptor or gentle-morselizer, facilitating tissue dislodgement or avulsion into the inner suction cannula 9A. As illustrated in FIGS. 6A through 6C3, the fenestrations are designed to extend both proximal and distal to the excursion of the inner cannula 9A. Thus, there is an additional area of the outer cannula which, being proximal to the tip of the reciprocating inner cannula at all times, serves solely as a disruptor, namely, approximately ⅙ of the aggregate fenestrated cross sectional area. There is also a considerable area, approximately ⅚ of the aggregate cross sectional fenestrated area which, being distal to the open-ended inner cannula 9A, at all times, is always in continuity with the vacuum source and aspirating tissue. Also, the central region of the aggregate cross sectional fenestrated area of the outer cannula 9B which will have a varying degree of vacuum applied during the reciprocation stroke.

### Specification of the First Illustrative Embodiment of the Twin-Cannula Assembly of the Present Invention

Referring to FIGS. 6A and 6D, a suction function is defined for the twin-cannula assembly 9—as being equal to the negative vacuum per cross sectional fenestrated area of outer cannula for three zones or surface areas (i.e. ZONE 1, ZONE 2 and ZONE 3) of fenestrated outer cannula and graphically display it as below. Normal atmospheric pressure is 14.7 lbs/in² or PSI, and a perfect vacuum would be 0 PSI. Vacuum pumps achieve ample suction, generally measured in mm of Hg., 29 in., but very far from perfect. A typical aspirator (i.e. vacuum pump) used in liposuction is the Wells Johnson Aspirator III which has one or more cylinder piston pumps in parallel for failure protection. As 51.7 mm Hg equal 1 PSI, a quality aspiration pump creates a vacuum in the vicinity of 0.56 PSI. Using the novel twin cannula assembly 9, this vacuum pressure level (i.e. 0.56 PSI) is applied to whatever portion of the fenestrated cross-sectional area of the outer cannula 9B is distal to the open tip of the inner cannula 9A, at the point of time the measurement is taken. For illustrative purposes, this suction function, so defined, will be used to illustrate the function of the improved twin-cannula design disclosed herein.

In the illustrative embodiment of the present invention, shown in FIGS. 6A through 6D, there are 3 slots 9C arranged in series, and one such slot arrangement is disposed at 120° angles on the distal end of the outer cannula. Thus, there are 9 equal-sized cross-sectional oval aspiration apertures or slots 9C potentially exposed to a vacuum pressure of 0.56 PSI. Also, for modeling purposes, it is assumed that these 9 oval-shaped aspiration slots are divided into thirds, such that there are 27 approximately-equal cross-sectional surface areas of suction pressure, formed about the fenestrated outer cannula 9B.

As shown in FIG. 6A, the fenestrated cross-sectional surface area of the outer cannula 9B is divided into three zones (i.e. ZONE 1, ZONE 2, and ZONE 3) reflecting continuity with the vacuum source. These three zones will be specified in greater detail below. Though this illustration is used with a cannula design featuring fenestrations in a 120° configuration, analogous discussions and calculations apply to single slot, 180° (two series of fenestrations), 90° (four series of fenestrations), 72° (five series of fenestrations), or even 60° (six series of fenestrations) oriented fenestrations, though there is a diminishing return as to the required metal to separate the individual fenestration apertures 9C with the preferred embodiment being three as described below.

ZONE 1 is defined as the proximal third portion of the most proximal slots 9C, over which optimal suction pressure (i.e. 0.56 PSI) cannot be achieved as these slots are never in continuity with that vacuum as the inner cannula open-ended lumen 9A remains distal to it. Thus, this 3/27 portion of the fenestrated outer cannula cross-sectional surface area is never exposed to any vacuum pressure at all, i.e. at sea level it remains at 14.7 PSI, and functions only as an irregular surface morselizer or fat disruptor. Abrasion of the tissue with this disruptor serves to dislodge and free fat for easy aspiration into the outer cannula fenestrations exposed to suction.

ZONE 2 is defined as the distal two-thirds of the proximal three circumferential slots 9C, over which the entirety of the three middle circumferential slots, and the proximal two-thirds of the most distal three circumferential slots (i.e. 21/28 of the fenestrated outer cannula cross-sectional surface area) is exposed (to a varying degree) to the applied vacuum of 0.56 PSI. Over this zone, an applied vacuum varies from 0 PSI (when the open-end of the distal inner cannula occludes them) to some maximal value of vacuum pressure when the inner cannula open-end 9A is proximal or immediately sub-adjacent to the fenestrated surface area of this Zone. At the maximal backward stroke, shown in FIG. 6B3, each of these imaginary one-third slot cross-sectional surface area divisions exerts a maximum suction force of 1/21×0.56 PSI. Expressed differently, if the size of the outer cannula 9B were such that their aggregate cross section were one square inch, each of these ⅓ portions of outer cannula slot would suck aspirated tissue in with a force of 1/21*0.56 lb or 0.027 lb. In this example, the force on this group of slot divisions would vary between 0 lbs suction and 0.27 lbs., or conversely experience an atmospheric pressure between 14.7 PSI and 14.43 PSI.

ZONE 3 is defined as the distal third of the most distal slots 9C, over which continuity is always retained with the applied vacuum as that portion of the distal slots is always distal to the open-ended inner cannula 9A. This 3/27 portion of the fenestrated outer cannula cross-sectional surface area is always exposed to a vacuum pressure of at least 0.56 PSI PSI allocated over each of the 3 always exposed areas equally or 0.19 PSI each. However, when the inner cannula retracts and exposes the middle selection of slot divisions to vacuum, the 0.56 PSI is then allocated over the surface area represented by 24/27 of the fenestrated surface area, so each ⅓ slot cross-sectional surface area sees 1/24 or 0.023 PSI. Hence in this illustration the force of suction varies between a minimum of 0.023 PSI and a maximum of 0.19 PSI.

The force of the vacuum experienced by each of these zones of outer cannula cross-sectional surface area (i.e. resulting suction function) will be graphically illustrated and described below for the novel twin cannula assembly design and configuration of the present invention.

### Specification of the Zonal Suction Function of the Twin-Cannula Assembly of the Present Invention

During tissue aspiration operations, the twin-cannula assembly 9 supports highly-effective surface areas of tissue aspiration about its three suction zones provided at the distal portion of the cannula, as illustrated in FIGS. 6A through 6C3. As illustrated in FIG. 6D, Zones 2 and Zones 3 support pulsatile vacuum forces (i.e. a very pulsatile suction function) which tends to disrupt, dislodge, and dislocate any temporary accumulations or conglomerations of more fibrous aspirate in the inner cannula 9A, where the lumen is narrower than the vacuum tubing, or elsewhere in the vacuum path between the aspiration instrument and the vacuum pump. It is appropriate, at this juncture, to further describe the function and operation of these three suction pressure zones supported at the distal portion of the twin-cannula assembly of the present invention 9, with reference to the Zonal Suction Function characteristic set forth in FIG. 6D for the illustrative embodiment of the twin-cannula assembly.

As illustrated in FIG. 6D, Zone 3, representing the most distal portion of the outer cannula, will always have suction and roughly one-third of the time will have the highest level of efficacy. Such forces over Zone 3 will never be “neutralized” by a surgeon's manual reciprocation (i.e. the surgeon moving the hand-piece forward synchronously as the hand piece is moving the cannula backwards and vice forward as might be favored by a rate of reciprocation roughly equal to a surgeon's habitual rate of manual stroking), and aspiration over Zone 3 will be pulsatile with or without his stroke, augmenting the suction function and aspiration rates. The rate of change of suction pressure, as a function of cannula stroke or time, is greater when the Zone 2 cross sectional area is exposed to pressure vacuum.

As illustrated in FIG. 6D, Zone 2, representing the middle one third of the distal portion of the outer cannula, will have varying degrees of vacuum pressure (i.e. force) between zero to the full vacuum, allocated over both Zone 2 and Zone 3 assuring that a very large area of tissue will be exposed to suction forces at any point in time, with more force delivered to Zone 2, some of the time.

The cross-sectional areas of Zone 3 and Zone 2 will see highest suction forces closest to the tip of the cannula during backstroke and/or forward stroke inner cannula operations when the inner cannula is closed to its full forward stroke position. The suction forces will drop off with distance along the proximal direction of the cannula. This suction profile characteristics are ideal for surgical as the surgeon accomplishes most tissue removal at the tip of the instrument, rather than along its length. This suction profile is also ideal for creating a smooth suction function without second derivative irregularities, as the advancing inner cannula will be exerting more suction as it truncates and lops of tissue protruding through the outer cannula fenestrations as it advances in a forward stroke.

During inner cannula backstroke movements, vacuum (i.e. suction) pressure will be dissipated over more fenestrations so it will allow tissue any tissue accumulated within the inner cannula to be aspirated down the tubing and evacuated from the inner cannula into the canister. The pulsatile force, the location of its applied forces, and the reciprocating inner cannula work in concert to achieve a maximal sustained rate of aspiration or suction function without stops and starts, accumulations and releases, uneven tissue removal, or unnecessary vibration.

Additional functional advantages are provided by the improved twin-cannula assembly of the present invention. Specifically, the herky-jerky vibration of the hand-piece, created by interval vacuum obstruction and its release, is also reduced by eliminating the interval obstruction and fat build-up during tissue aspiration. This improvement reduces the surgeon's risk of repetitive stress injury to his or her wrists, elbows and shoulders (i.e. carpal tunnel syndrome, “tennis elbow” or lateral epiphysitis, or bicipital tendonitis). This improvement also reduces patient discomfort when aspiration is performed under local anesthesia, because the patient is much more likely to be aware of such sudden jerks and starts. This improvement also reduces the stresses on whatever means of actuation are used to effect inner cannula reciprocation, as any system subject to start and stop motion, with unbalanced forces, is subject to more wear and tear than a system functioning in equilibrium at a steady and even rate of operation.

Second Illustrative Embodiment of the Tissue Aspiration Instrumentation System of the Present Invention, Provided with a New and Improved RF-Based Bipolar Electro-Cauterizing Twin Cannula Assembly

In FIG. 7A through 10D, a second illustrative embodiment of the present invention is shown comprising: a hand-supportable tissue aspiration instrument 50 having (i) a hand-supportable housing 2 with a stationary tubing connector 4 provided at the rear of the housing and receiving a length of flexible tubing connected to a vacuum source, and (ii) a twin-cannula RF-based bipolar electro-cauterizing assembly 9′ having an inner cannula 9A′ coupled to a pneumatically-powered cannula drive mechanism disposed within the hand-supportable housing and powered by a source of pressurized air or other gas, while its fenestrated outer cannula 9B′ is releasably connected to the front portion of the hand-supportable housing 2. As shown, outer cannula 9B′ is installed over the inner cannula 9A′ and connected to the front-end portion of the hand-supportable instrument housing 2, in a stationary manner.

As shown in FIG. 7B, the air-powered tissue aspiration instrument 50 comprises a quick connect plug and multi-core cable assembly 19 is provided on the rear portion of the hand-supportable housing, for supporting two gas lines and three electric wires 20 between the instrument 2 and its controller 21 in a single bundle, as taught in U.S. Pat. No. 7,381,206 to Cucin, incorporated herein by reference, but without the extra two widely separated RF leads provided for electro-cautery and without the extra three pins for LV control circuits.

As shown in FIG. 7C, the second illustrative embodiment of the tissue aspiration instrumentation system 450 comprises a hand-supporting housing 2, in which its cylindrical guide tube 1 and an air-powered driven mechanism are installed, while its cannula base portion 13′, inner cannula 9A′ and cannula lock nut 10 are shown disassembled outside of the hand-supportable housing 2. Preferably, the inner cannula component 9A′ is made from a suitable plastic material, as will be described in greater detail hereinbelow.

FIG. 7D shows the outer cannula 9B′ which is installed over the inner cannula 9A′ when the inner cannula 9A′ is coupled to the inner cannula base portion 13′ via its leur-lock connector assembly, shown in FIG. 7C. As shown in FIG. 7D, outer cannula 9B′ comprises: a base portion 9B1′ having internal threads that screw over matching threads on cannula lock nut 10, threaded into the front portion of the hand-supportable housing 2; and a lumen portion 9B2′ extending from base portion 9B1′ at its proximal end, and having a fenestrated distal portion having multiple sets of aspiration apertures 9C′ and terminated with a blunt bullet-tip nose. Preferably, the outer cannula component 9B′ is made from a stainless steel, or other suitable material, as will be described in greater detail hereinbelow.

As shown in FIG. 8A, the hand-supportable tissue aspiration instrumentation system 50 is configured with its aspiration source, its controller and pneumatic power source 21, and multi-core cable assembly 20. FIG. 8A also reveals a number of important features of this illustrative embodiment of the tissue aspiration instrument, namely: that the solitary reciprocating inner cannula 9A has a leur-lock fitting 15 to mate to a leur-lock fitting 16 on the hollow inner cannula base portion 13′, externally to the hand-supportable housing 2; that magnet 8 is affixed to cannula base portion 13′ using a screw-on nut 5; that front and rear gas tubes 17 and 18 run to from the front of the housing to the rear multi-core quick connect plug 19; that the quick connect multi-core plug 19 connects to multi-core cable containing two fluidic (gas) channels 20, and at least three low-voltage electrical circuits; that cable 20 runs to the controller 21, within which the gas channels directly attached to the compressed gas source (not shown); that the front and rear Hall sensors 22 and 23 are provided within the hand-supportable housing, for detecting the excursion of the hollow inner cannula base portion 13′ within the cylindrical guide tube 1; front and rear flat sealing washers 6 and 7 are provided for slidably supporting the cannula base portion 13′ along the cylindrical guide tube 1; and threaded chamber cover (i.e. cannula lock nut) 10 is provided with a hole, through which the inner cannula 9A protrudes.

As shown in FIG. 8B, the controller (and air-power supply) console 21 comprises a number of components, namely: an ADC receiving signals generated by the front and rear Hall-effect cannula base position sensors installed within the hand-supportable housing of the instrument; a LCD panel; communication ports; LED indicators; and panel membrane switches supported on the controller console housing; digital signal processor (DSP); and a DAC and proportional valve contained within the controller console housing, and supplying gas tubes (via the multi-code cable assembly); and ports for receiving a supply of pressurized gas, for controlled supply to the cannula drive mechanism of this embodiment of the present invention. The details of this controller 21 can be found in U.S. Pat. No. 7,381,206 to Cucin, incorporated herein by reference.

As shown in FIG. 8B, the air-powered tissue aspiration instrument 50 further comprises: a single-button quick connect plug 19, and associated multi-core cable assembly 20 is provided on the rear portion of the hand-supportable housing. The function of the multi-core cable assembly is to support at least two gas lines and at least three electric wires between the instrument and its controller 21 in a single bundle, as taught in U.S. Pat. No. 7,381,206 to Cucin, incorporated herein by reference, with an extra two widely separated RF leads provided for electro-cautery and without the extra 3 pins for low voltage control circuits. Also, in this embodiment, the walls of at least the front (pneumatic) chamber portion of housing should be made from a non-magnetizable metal (e.g. SS 304) or other material that will support the necessary gas pressure of actuation (e.g. ˜100 PSI).

Also, the Hall effect sensors installed in the housing sense the position of the cannula base portion by sensing the magnetic field of its magnetic ring 8. As the cannula base portion 13′ reciprocates within the cylindrical guide tube 1′, the aspiration/vacuum tubing connected to the barb connector on the stationary tubing connector, remains stationary and thereby preventing any jerking action on the surgeon's hands which can cause carpal tunnel syndrome. Also, the inner and outer cannulas 9A, 9B are provided with leur-lock fittings 15, 16, while the cannula base portion is provided as a sterile single-use disposable item, made from plastic or metal, and having a low cost magnet and silicone washers to provide fluid seals between the cannula base portion and the cylindrical guide tube within the hand-supportable housing 2.

In this illustrative embodiment, there must be an air-tight seal around the (inner) cannula as it exits the pneumatic cylinder/chamber so that air pressure is not lost to the ambient environment. Any air will escape that seal and harmlessly vent into the air as the pneumatic cylinder is separate from the aspiration path (lumen) within the inner cannula. There must be a generous vent formed in the outer cannula base portion to make sure that any escaping air from the pneumatic chamber seal does not cross the space between the outer and inner cannulas into the patient during instrument operation. A second sealing washer distal to that vent may be employed for extra patient safety.

### Specification of the Second Illustrative Embodiment of the Twin-Cannula Assembly of the Present Invention

FIG. 9A shows the base portion of the inner cannula 9A′ component used in the bipolar electro-cauterizing cannula assembly 9′ shown in FIG. 7A. FIG. 9C shows the plastic inner cannula tube 9A2′ with embedded wire conductors 9A3′ for conducting RF power signals to the distal portion of open inner cannula. In this embodiment of the cannula assembly 9′, one or more (six shown) coaxially co-extruded wires conduct one side of the RF bipolar cautery circuit. A circumferential conductive ring can be crimped on the proximal end of the inner cannula, to establish electrical continuity with each conductive wire, so that multiple (i.e. 3) sets of neighboring wires provide an independent RF circuit, and each side of the RF circuit is connected to one RF input lead or contact formed on the proximal end of the inner cannula, so that a pair of brushes (or spring-loaded contacts) can conduct RF single input into the RF circuits as the inner cannula reciprocates within the stationary outer cannula. The outer cannula is preferably coated with PFA (i.e. electrically insulating coating, except at the under-surface of its hub, which is in contact with a spring-loaded contact. Each RF circuit is closed as aspirated tissue bridges the gap and closes the circuit between the ends of pairs of neighboring co-extruded inner cannula wires.

Alternatively, the bipolar electro-cauterizing cannula assembly 9′ can be constructed by embedding wire conductors 9A3′ within a plastic inner cannula 9A′, to form one half of the RF circuit, and using a conductive outer tube, with a PFA coating on the inside surface to prevent electrically shorting with the inner cannula. A circumferential ring can be crimped onto the base portion of the plastic inner cannula to establish contact with the conductive wires and the crimped ring can be placed in contact with a first spring loaded contact to supply the first side of the RF power signal, whereas a second spring-loaded contact establishes electrical contact with an exposed region of the outer cannula to supply the other side of the RF power signal. The bipolar RF signals can be supplied to the pair of spring-loaded contacts by electrical wiring or other known means and ways known in the art. The circuit is then closed as aspirated tissue bridges the gap and closes the RF circuit formed between (i) the ends of any of the co-extruded inner cannula wires, and (ii) the inside surface of the coated outer cannula, or any of the exposed edges of the outer cannula fenestrations.

FIG. 10A shows the fenestrated distal tip portion of the twin-cannula assembly 9′ shown in FIG. 7A, indicating the location of its three primary zones of vacuum pressure along the distal portion thereof, namely ZONE 1, ZONE 2 and ZONE 3. FIG. 10B1 shows RF bipolar electro-cautery twin-cannula assembly 9′ removed from the hand-supportable tissue aspiration instrument 50 shown in FIG. 7A, for purposes of illustration. FIG. 10B2 shows the distal portion of the twin-cannula assembly illustrated in FIG. 10B1, when its open-ended inner cannula is slidably disposed at an extreme backward most position within the fenestrated (i.e. apertured) outer cannula, terminated in a blunt, bullet-nose shaped distal tip portion 9B4′. FIG. 10C2 shows the distal portion of the twin-cannula assembly 9′ illustrated in FIG. 10C1, when its open-ended inner cannula is slidably disposed at an extreme backward most position within the fenestrated outer cannula. Except for its bipolar cauterization functions, the twin-cannula assembly 9′ is similar to the twin-cannula assembly 9 described above, and shall not be repeated to avoid unnecessary redundancy. However, the bipolar cauterization functions of twin-cannula assembly 9′ will benefit from some additional specifications.

### Specification of Bipolar Electro-Cautery Circuits Embodied in the Twin-Cannula Assembly

In one embodiment of the electro-cauterizing cannula assembly 9′ described above, coextruded conductors are located in a disposable plastic cannula 9A′ at either edge of one or more holes in the inner cannula which register with the outer cannula slot. The RF circuit can be closed by one pole being located on either side of the inner cannula hole, or by one side on the inner cannula wires and the other pole being located at the outer cannula.

In another embodiment, a disposable electro-cauterizing inner cannula can be used which carries both sides of RF circuit. While this design has the benefit of carrying both sides of the RF circuit so the outer cannula can be uncoated metal or inexpensive plastic, making the hub connections and assuring exposure of the wires at the sides of the inner cannula aperture create significant manufacturing hurdles. In such embodiments, extrusion angles specific to each cannula size must be designed with tight angular tolerances±1.0° and the holes cut to even tighter tolerances±0.5°.

In yet another alternative embodiment, a plastic inner cannula can be co-extruded with six conductors, as shown in FIG. 9C. This design offers a number of functional and production advantages when implementing bipolar electro-cautery functionalities. For example, many manufacturers are now capable of standard co-extrusions for four or six conductors. Thus, if only one side of the RF circuit is carried on the inner cannula, and the circuit is closed using metal within the walls of the outer cannula or a metal outer cannula, them the base of a plastic cannula having six coaxially extruded conductors (such as 35N DFT 28% Ag wire) can be crimped with a band having multiple serrations to assure making contact with the conductors within the outer cannula wall. These conductors can be simply exposed at the open distal end of the inner cannula so that one or more of them makes contact with the stalk of fat globules suctioned within the inner cannula lumen or a serrated circumferential ring similar to the one used to make contact at the hub.

Notably, this design eliminates alignment issues for bipolar electro-cautery, as well as stationary axis requirements for the inner cannula mount, with possibility of a smaller inner cannula mount footprint, and the elimination of the necessity of hand piece chamber access with a panel or door.

The vacuum pressure versus time graph characteristics shown in FIG. 10D illustrate the vacuum strength over the three primary zones along the twin-cannula assembly 9′, during a complete inner-cannula reciprocation cycle, providing a zonal suction function specifying the performance of the fat tissue aspiration instrument used with the twin-cannula assembly. Such characteristics are similar to those shown in FIG. 6D.

Third Illustrative Embodiment of the Tissue Aspiration Instrumentation System of the Present Invention, Provided with a New and Improved Twin Cannula Assembly

In FIGS. 11A through 12D, a third illustrative embodiment of the tissue aspiration instrumentation system 60 is shown comprising a hand-supportable tissue aspiration instrument having an interior payload (i.e. bay) compartment 61 with a hinged door panel 62 for loading the inner cannula 9A″ through its bay and out a front opening formed in the housing 2″, and then connecting the flexible vacuum tube to the barbed end connector on inner cannula base portion 9A1″ (and out a port formed in the rear portion of the housing). Also, a pneumatically-powered (or electromagnetically-powered) cannula drive mechanism 63 is installed within the housing, for driving the electro-cauterizing inner cannula 9A″ within a stationary outer cannula 9B″, that is releasably mounted to front portion of the hand-supportable housing 2″, while the instrument is controlled by a control console 21 as generally described in FIG. 8B.

FIG. 11B illustrates the distal portion of the twin-cannula assembly 9″ connected to the air-powered tissue aspiration instrument 60 shown in FIG. 11A, whereas FIG. 11C shows a disassembled inner and outer cannula components of the twin-cannula assembly 9″. Except for the hollow inner cannula base portion 9A″, inner and outer cannula components shown in FIGS. 11B through 11D are essentially the same as shown in FIGS. 3B, 4A, 6B1.

FIGS. 12A through 12D describes the functional performance of the twin-cannula assembly 9″ shown in FIGS. 11A through 11D, which is similar to the twin-cannula assembly 9 described above, and shall not be repeated to avoid unnecessary redundancy.

Fourth Illustrative Embodiment of the Tissue Aspiration Instrumentation System of the Present Invention, Provided with a New and Improved RF-Based Bipolar Electro-Cauterizing Twin Cannula Assembly

In FIGS. 13A through 13D, a fourth illustrative embodiment of the tissue aspiration instrumentation system 70 is shown comprising: a hand-supportable tissue aspiration instrument having an interior payload (i.e. bay) compartment 71 with a hinged door panel 72 for loading the inner cannula 9A′″ through its bay and out through a front opening formed in the housing 2′″; and a flexible vacuum tube connected to the barbed connector on the inner cannula base portion 9A1′″ shown in FIG. 14, and passing through a rear opening formed in the rear portion of the housing. Also, the tissue aspiration instrument 70 further comprises: a pneumatically-powered (or electromagnetically-powered) cannula drive mechanism 73 is installed within the housing, for driving the RF bipolar electro-cauterizing inner cannula 9A′″ within a stationary outer cannula 9B′″, that is releasably mounted to front portion of the hand-supportable housing 2′″, while the instrument is controlled by a control console 21, as generally described in FIG. 8B.

FIG. 13B illustrates the distal portion of the twin-cannula assembly 9′″ connected to the air-powered tissue aspiration instrument 70 shown in FIG. 13A, whereas FIG. 13C shows a disassembled inner and outer cannula components of the bipolar electro-cauterizing twin-cannula assembly 9′″. Except for the hollow inner cannula base portion 9A1′″ shown in FIG. 14A, which mounts within the interior chamber of the hand-supportable housing 2, rather than external thereto, the inner and outer cannula components 9A′″ and 9B′″ shown in FIGS. 14A through 14C are essentially the same as the inner and outer cannula components 9A′ and 9B′ shown in FIGS. 9A, 9B and 9C.

FIG. 14A shows the base portion of the inner cannula 9A′″ component used in the bipolar electro-cauterizing cannula assembly 9′″ shown in FIG. 13A. FIG. 14C shows the plastic inner cannula 9A2′″ with embedded wire conductors 9A3′″ for conducting RF power signals to the distal portion of open inner cannula. In this embodiment of the cannula assembly 9′″, one or more (six shown) coaxially co-extruded wires conduct one side of the RF bipolar cautery circuit. A circumferential conductive ring can be crimped on the proximal end of the inner cannula, to establish electrical continuity with each conductive wire, so that multiple (i.e. 3) sets of neighboring wires provide an independent RF circuit, and each side of the RF circuit is connected to one RF input lead or contact formed on the proximal end of the inner cannula, so that a pair of brushes (or spring-loaded contacts) can conduct RF signal input into the RF circuits as the inner cannula reciprocates within the stationary outer cannula. The outer cannula is preferably coated with PFA (i.e. electrically insulating coating, except at the under-surface of its hub, which is in contact with a spring-loaded contact. Each RF circuit is closed as aspirated tissue bridges the gap and closes the circuit between the ends of pairs of neighboring co-extruded inner cannula wires.

Alternatively, the bipolar electro-cauterizing cannula assembly 9′″ can be constructed by embedding wire conductors 9A3′″ within a plastic inner cannula 9A′″, to form one half of the RF circuit, and using a conductive outer tube, with a PFA coating on the inside surface to prevent electrically shorting with the inner cannula. A circumferential ring can be crimped onto the base portion of the plastic inner cannula 9A′″ to establish contact with the conductive wires and the crimped ring can be placed in contact with a first spring loaded contact to supply the first side of the RF power signal, whereas a second spring-loaded contact establishes electrical contact with an exposed region of the outer cannula to supply the other side of the RF power signal. The bipolar RF signals can be supplied to the pair of spring-loaded contacts by electrical wiring or other known means and ways known in the art. The RF circuit so formed is then closed, electrically, as aspirated tissue bridges the gap and closes the RF circuit formed between (i) the ends of any of the co-extruded inner cannula wires, and (ii) the inside surface of the coated outer cannula, or any of the exposed edges of the outer cannula fenestrations.

FIGS. 15A through 15D describe the operation and suction function of the electro-cauterizing twin-cannula assembly 9′″ which is similar to the operation and suction function of electro-cauterizing twin-cannula assembly 9′.

### Fifth Illustrative Embodiment of the Tissue Aspiration Instrumentation System of the Present Invention

In FIGS. 16A through 29B3, a fifth illustrative embodiment of the tissue aspiration instrumentation system 80 is shown comprising a hand-supportable tissue aspiration instrument 81 (i.e. powered hand-piece) equipped with a fifth illustrative embodiment of the twin-cannula assembly of the present invention 9″″ having an open-end type inner cannula 9A″″ that mounts within an outer cannula 9B″″ releasably connected to the front portion 81A of the hand-supportable instrument (i.e. hand-piece) 81. As will be described in greater detail hereinafter, the open-ended type inner cannula 9A″″ is driven by an electromagnetic cannula driven mechanism 83 contained with the hand-supportable housing 100 of the hand-piece portion of the instrument 81.

As shown, the rear portion of the instrument 81B supports a stationary aspiration tubing connector 85 that extends along the longitudinal axis 86 of the hand-supportable device. As shown, the tubing connector 85 is connected to a vacuum pump device 87 by a suitable piece of flexible aspiration tubing 88.

As shown in FIG. 16B, the rear side 100A of the hand-supportable housing 100 provides: (i) a control potentiometer 90 that allows the surgeon to manually set the rate of reciprocation of the inner cannula 9A″″ within the stationary outer cannula 9B″″; and (ii) a power input port 91 for connecting a flexible power cord 92 that terminates in a power adapter 93 plugged into a standard AC power receptacle. The power adapter 93 receives 120 Volt AC power from the AC receptacle, conditions the 120 Volt input AC power signal, and then supplies the conditioned AC output voltage signal to drive the electro-magnetic coils 112A, 112B and 112C, as shown in FIGS. 21 and 22.

In FIG. 16C, the assembled twin-cannula assembly 9″″ is shown removed and detached from its hand-piece device 81. In FIG. 16C, the twin-cannula assembly 9″″ is shown disassembled and detached from the hand-piece 81. As shown in FIG. 16D, the inner cannula 9A″″ has an inner base portion connector 9A1″″ mounted at its proximal end for coupling to its disposable inner cannula base portion 94 (e.g. by a twist-lock connector, leur-lock fittings, etc), an open-ended type aspiration aperture 9A2″″ formed at its distal end, and a lumen portion disposed therebetween having an outer diameter (OD) that fits within the inner diameter (ID) of the stationary outer cannula 9B″″.

As shown in FIG. 16D, the outer cannula 9B″″ has an inner cannula base portion 9B1″″ that releasably couples to an outer cannula mount 96 secured to a clip-on housing nose cover 95, that releasably connects to the housing body 100, as will be described in greater detail below. The outer cannula 9B″″ also has a fenestrated distal portion having multiple zones of outer aspiration apertures 9C″″, as previously described in detail with respect to the other illustrative embodiments of the twin-cannula assembly 9 through 9″″.

It is appropriate at this juncture to discuss how the twin-cannula assembly 9″″ is attached and detached from the hand-supportable instrument housing 81, in accordance with the principles of the present invention.

As shown in FIG. 17A, the fenestrated outer cannula 9B″″ is rotated relative to the hand-supportable housing to unlock the outer cannula base portion 9B1″″ from the outer cannula mount portion 96 secured to the clip-on housing nose cover 95. Once unlocked, the outer cannula is slid off the inner cannula. Then, the clip-on housing nose cover 95 can be removed off the front-end of the hand-supportable housing 100, by (i) depressing inwardly on the semi-spherical projections 98A and 98B formed on side projections 99A and 99B (that extend in a forward direction from the hand-supportable housing 100 as shown in FIGS. 20, 23 and 24) so that (ii) projections 98A and 98B release from holes 95A and 95B, respectively, formed in the clip-on housing nose cover 95, as shown in FIG. 24.

FIG. 17B shows the fenestrated outer cannula 9B″″ removed off the attached inner cannula 9A″″, and the clip-on housing nose cover 95 removed off the front-end of the hand-supportable housing 100A. Also, the inner cannula 9A″″ and inner base portion 94 subassembly is then removed from the front end of the hand-supportable housing 100A.

FIG. 17C shows the fenestrated outer cannula 9B″″ removed off the attached inner cannula, and the clip-on housing nose cover 95 removed off the front-end of the hand-supportable housing 100A. Also, the inner cannula 9A″″ and inner base portion 94 subassembly is removed from the front end of the hand-supportable housing 100A. Then the inner base portion 94 is decoupled from the disposable inner cannula 9A″″.

As shown in FIGS. 17D and 18A, the inner cannula base portion 94 comprises the following components: a hollow base portion tube 94A having a first end opening 9H provided with a leur or like connector, and a second end opening 9I communicates with the rear mounted tube connector 85 when tube 94 is installed in housing 100; a first fluid seal 94B mounted about the tube 94A closer towards the first end opening 94, but a distance sufficient to achieve the desired inner cannula excursion during instrument operation; a permanent magnetic ring 94B, slide over the first end opening 94H, and pushed against the first fluid seal 94B; a set of threads 94G spaced apart from the first fluid seal 94B by a distance slightly greater than the thickness of the permanent magnetic ring 94D; a second fluid seal 94C for sliding over the first end opening 94H, positioned against the permanent magnetic ring 94D and securely threaded in place, over threads 94G; and a pair of return springs 94E and 94F slid over the first and second end openings 9H and 9I, and disposed against the first and second fluid seals 94A and 94B, respectively.

FIGS. 17E and 17F shows the proximal end portion of the inner cannula 9A″″ adapted to couple with the inner cannula base portion 94 described in detail above. As shown, the proximal end portion has an adapter coupling 103 attached to the proximal end of the inner cannula lumen 9A4″″, comprising: a first annular portion 103A having an outer diameter (OD) slightly less than the inner diameter (ID) of the cylindrical guide tube 106 mounted within the hand-supportable housing 100, allow the adapter coupling 103 to slide within the guide tube 106 during inner cannula reciprocation operations; a second annular portion 103B having an outer diameter (OD) slightly less than the inner diameter (ID) of the first end opening 9H formed in the inner cannula base portion 94 so as to mount within the first end opening 9H, and allow the adapter coupling 103 to slide within the guide tube 106 during inner cannula reciprocation operations; and flanges 103C and 103D formed on the end of the second annular portion 103B, to allow the inner cannula to couple with the first end opening 9H, by way of a turning action; and a proximal end opening 9A5″″ allowing the inner lumen to communicate with the hollow inner cannula base portion 94 upon the coupling of these two components, as shown in FIG. 18B.

FIG. 18B shows the inner cannula 9A″″ with its hollow inner base portion 94 coupled thereto. In this assembled state, the inner cannula subassembly can be loaded into the front loading portal 100C of the hand-piece 81, as shown in FIG. 17B.

FIG. 19 shows the hand-supportable housing 100 and its rear end opening 100D, into which the cannula guide tube and aspiration tubing connector assembly 108 of FIG. 21 are slid and mounted during assembly.

FIG. 20 shows the cannula guide tube and aspiration tubing connector 104, preferably a single-piece structure molded from plastic material, comprising: a cannula guide tube portion 105 and an aspiration tubing connector portion 106, each having hollow centers 105A and 106A respectively, and being aligned along a common longitudinal axis 107 and being in fluid communication with each other; an electromagnetic coil-winding support structure 108 formed by four spaced-apart annular flanges 108A, 108B, 108C and 108D extending traverse to the longitudinal axis of the cannula guide tube portion 105 and defining three annular regions 109A, 109B and 109C, about the cannula guide tube 105 where electromagnetic coiling windings 112A, 112B and 112C, can be wound respectively, during manufacture; and a circular-shaped rear housing plate 110 for mounting rotatable potentiometer 90, and power input port connector 91 connected to power adapter (and drive signal generator) 93, by way of flexible cable 92, schematically illustrated in FIG. 22.

In a first illustrative embodiment, form factor of the AC/DC power adapter 93 of FIG. 22 can be a wall plug having an integrated two-prong electrical plug for plugging in a standard AC power wall receptacle. The AC/DC circuitry and drive signal generator 90A, can be realized using timing circuits, a RC network and the like, and contained within the wall plug housing, and its flexible power cable 92 can be provided with a plug connector that interfaces with plug connector 91 mounted on the rear housing panel 110.

In an alternative embodiment, the form factor for AC/DC power adapter device 93 can be a power block module, wherein a length of power cord with an AC power plug extends from a power block module containing AC/DC circuitry and drive signal generator 90A, and having a flexible power cable 92 with a plug connector that interfaces with plug connector 91 mounted on the rear housing panel 110.

As shown in FIG. 21, an electromagnetic-coil based cannula drive mechanism 112 for mounting within the hand-supportable housing 100, comprises: the cannula guide tube and aspiration tubing connector 104 shown in FIG. 19; and three electromagnetic coiling windings 112A, 112B and 112C wound on the annular regions 109A, 109B and 109C of the electromagnetic coil-winding support structure, respectively, as shown. While not shown in FIG. 21, a barbed tubing connector 85 is threaded on the end of the aspiration tubing connector 106, and allows for piece of flexible aspiration tubing to be pushed over the barbs and securely connected thereto, without easy disconnection. As shown in the schematic circuit diagram of FIG. 22, the electromagnetic coil drive circuit employed in the twin-cannula tissue aspiration instrument shown in FIGS. 16A through 16D, comprises: front electromagnetic coil 112A, middle electromagnetic coil 112B, and rear electromagnetic coil 112C, wired as shown; potentiometer 90, and power input port 91. As shown, the potentiometer 90 is the rotary type, so that a knob can be used to allow the surgeon to rotate the same and adjust the reciprocation rate of inner cannula (e.g. from about 500 reciprocation cycles per minute (i.e. CPM) to about 1000 CPM for about ¼ to ⅜ inch stroke, or from about 200 CPM to about 400 CPM for a 1.50 to 2.25 inch stroke).

As shown in FIG. 23, the electromagnetic-coil based cannula drive mechanism 112 in assembled form is slide through the rear opening of the hand-supportable housing, so that the front portion of the cannula guide tube 105A is inserted with central hole 100C formed in the front portion 100A of the hand-supportable housing. When fully inserted, the rear-housing panel 110 closes off the rear end opening of the housing, and can be secured in place by glue, ultrasonic welding or other techniques known in the plastics art.

When completely assembled as shown in FIG. 24, the hand-supportable housing provides (i) a front opening 115, as shown in FIGS. 17B and 27B, and defined by the front opening 105A of the cannula guide tube 105, and (ii) a barbed aspiration tubing connector 85, both coaxially located along the common longitudinal axis 107. The assembled electromagnetically driven hand-piece portion and twin-cannula assembly components of the instrument 80 can be sterilized in an autoclave, in a conventional manner. The hollow inner cannula base portion 94 can be made as a disposable component, or made for sterilization in an autoclave and reused among patients.

FIG. 25 shows the outer cannula 9B″″ detached from the inner cannula of the tissue aspiration instrument shown in FIG. 24. As shown, the distal end is provided with fenestrations 9C″″ as described hereinabove with respect to the other illustrative embodiments. As shown in FIGS. 26A and 26B, the proximal end is provided with an outer cannula base portion 9B1″″ having a thin cylindrical lock element (i.e. pin) 9B4″″ that is threaded through the side wall of the base portion cup portion 9B1″″ so that a small interior piece thereof extends into the interior portion of the base cup portion 9B1″″ and rides within and locks into the L-configured lock groove (or track) 96A formed in the outer cannula mount 96. The manner in which the lock pin 9B4″″ locks into L-shaped lock groove 96A on the outer cannula mount 96 will be illustrated and described in the twin-cannula assembly and installation sequence set forth in FIGS. 27A through 27I, and described hereinbelow.

FIG. 27A shows the disassembled primary components of the twin-cannula tissue aspiration instrument shown in FIG. 16A, namely: powered hand-piece 800 containing the cannula drive mechanism 112 contained within the hand-supportable housing 100; clip-on housing nose portion 95 with outer cannula mount 96; hollow inner cannula base portion 94 with permanent ring magnet 94D; inner cannula 9A″″; and fenestrated outer cannula 9B″″.

FIGS. 27A and 27B illustrate the next step in the cannula assembly process, wherein the inner cannula 9A″″ is coupled to its inner cannula base portion 94 by inserting (i) the inner base portion connector 9A1″″ on the proximal end of the inner cannula, into (ii) the front end opening 94H of the disposable inner cannula base portion 94.

FIGS. 27B and 27C illustrate the next step in the cannula assembly process, wherein the coupled inner cannula and inner cannula base portion is installed within the front central opening 115 of hand-piece component of the instrument 80.

FIGS. 27C and 27D illustrate the next step in the cannula assembly process, wherein the clip-on housing nose cover 95 is slid over the installed inner cannula and clipped-on to the hand-supportable housing by projections 98A and 98B popping through holes 95A and 95B, respectively, formed in the clip-on housing nose cover 95.

FIGS. 27D and 27E illustrate the next step in the cannula assembly process, wherein the outer cannula 9B″″ is being slid over the installed inner cannula, in preparation of locking the outer cannula to the outer cannula mount 96 fixed to the installed clip-on housing nose cover 95.

FIGS. 27F and 27G illustrate the next step in the cannula assembly process, wherein the outer cannula 9B″″, installed over the inner cannula, is pushed onto the outer cannula mount 96 so that interior portion of the lock pin 9B4′″″ slides into the groove 96A, and then the outer cannula base portion 9B1′″″ is rotated counter-clockwise into its locked position (by applying torque on the long exterior portion of lock pin 9B4″″ to cause outer cannula rotation). At this stage, the twin-cannula assembly 9″″ is completely installed and the instrument is ready for operation.

When it is time to remove the twin-cannula assembly from the hand-supportable housing, FIGS. 27H and 27I illustrate the next steps in the cannula disassembly process. As shown in FIG. 27H, the outer cannula 9B″″, installed over the inner cannula, is rotated clock-wise into its un-locked position, and then slid off the installed inner cannula, as shown in FIG. 27I. Then, the clip-on housing nose cover 95 is un-clipped and removed off the front portion of the hand-supportable housing 100, and then the outer cannula and inner cannula base portion assembly is slid out of and removed from the front loading (i.e. inner cannula guide tube) within the hand-supportable housing. Thereafter, the hand-piece and cannula components can be sterilized in an autoclave device in a manner well known in the surgical arts.

FIG. 28 shows the fenestrated distal tip portion of the twin-cannula assembly employed in the instrument of FIG. 16A, indicating the location of its three primary zones of vacuum pressure along the distal portion thereof, namely ZONE 1, ZONE 2 and ZONE 3.

FIGS. 29A1 through 29A4 show the twin-cannula assembly 9″″ removed from the tissue aspiration instrument of FIG. 16A, and configured when its open-ended inner cannula 9A″″ slidably disposed at an extreme backward most position, within the fenestrated outer cannula 9B″″. The operation of twin-cannula assembly 9″″ is similar to twin-cannula assemblies of the present invention shown and described hereinabove.

FIG. 29B1 through 29B4 show the twin-cannula assembly 9″″ removed from the tissue aspiration instrument of FIG. 16A, and configured when its open-ended inner cannula 9A″″ slidably disposed at the end of the forward stroke position, within the fenestrated outer cannula 9B″″.

The vacuum pressure versus time graph characteristics shown in FIG. 29C illustrate the vacuum strength over the three primary zones along the twin-cannula assembly 9″″, during a complete inner-cannula reciprocation cycle, providing a zonal suction function specifying the performance of the fat tissue aspiration instrument used with the twin-cannula assembly. Such characteristics are similar to those shown in FIG. 6D.

The twin-cannula assembly 9″″ described above can be readily modified to support bipolar RF-based electro-cauterization. To do so will involve practicing either of the techniques described above in connection with twin-cannula assemblies 9′ and 9′″.

Specifically, in a first illustrative embodiment, the inner cannula 9A″″ can be made from plastic tube embedded wire conductors for conducting RF power signals to the distal portion of the end opening of the inner cannula. In this RF embodiment of the cannula assembly 9″″, one or more (six shown) coaxially co-extruded wires conduct one side of the RF bipolar cautery circuit. A circumferential conductive ring can be crimped on the proximal end of the inner cannula, to establish electrical continuity with each conductive wire, so that multiple (e.g. 3) sets of neighboring wires provide an independent RF circuit, and each side of the RF circuit is connected to one RF input lead or contact formed on the proximal end of the inner cannula, so that a pair of brushes (or spring-loaded contacts) can conduct RF signal input into the RF circuits as the inner cannula reciprocates within the stationary outer cannula. The outer cannula is preferably coated with PFA (i.e. electrically insulating coating, except at the under-surface of its hub, which is in contact with a spring-loaded contact. Each RF circuit is closed as aspirated tissue bridges the gap and closes the circuit between the ends of pairs of neighboring co-extruded inner cannula wires.

In an alternative RF embodiment of twin-cannula assembly 9″″, the bipolar electro-cauterizing cannula assembly can be constructed by embedding wire conductors within a plastic inner cannula tube to form one half of the RF circuit, and using an electrically-conductive conductive outer tube, with a PFA coating on the inside surface to prevent electrically shorting with the inner cannula. A circumferential ring can be crimped onto the base portion of the plastic inner cannula to establish contact with the conductive wires and the crimped ring can be placed in contact with a first spring loaded contact to supply the first side of the RF power signal, whereas a second spring-loaded contact establishes electrical contact with an exposed region of the outer cannula to supply the other side of the RF power signal. The bipolar RF signals can be supplied to the pair of spring-loaded contacts by electrical wiring or other known means and ways known in the art. The circuit is then closed as aspirated tissue bridges the gap and closes the RF circuit formed between (i) the ends of any of the co-extruded inner cannula wires, and (ii) the inside surface of the coated outer cannula, or any of the exposed edges of the outer cannula fenestrations.

### Sixth Illustrative Embodiment of Two-Cannula Assembly for the Tissue Aspiration Instrumentation Systems of the Present Invention

As shown in FIG. 16, a curved outer cannula component 9B′″″ is provide for use with any of the tissue aspiration instruments of the illustrative embodiments employing a flexible plastic inner cannula 9, 9A′, 9A″, 9A′″ and 9A″″ in accordance with the principles of the present invention. Preferably, the curved outer cannula 9B′″″ is realized from suitable stainless steel, or at a disposable plastic material with sufficient stiffness.

As shown in FIG. 16, the outer cannula has the same fenestrations 9C′″″ at the distal tip portion, as described hereinabove, while the inner cannula has an open-end type aspiration opening (i.e. aperture), as hereinfore described as well. Using an open-ended inner cannula 9A, 9A′, 9A″, 9A′″ or 9A″″ with this curved fenestrated outer cannula design 9B′″″, allows very thin and inexpensive FEP plastics to be used to construct very thin inner cannulas, having minimally thick walls. This is possible because such a plastic inner cannula will be supported by the rigid thicker outer cannula, whether made of metal or plastic, thereby eliminating concerns about inner cannula inner diameter (ID) restrictions relating to the use of plastic.

Also, the open-ended inner cannula design eliminates alignment issues as there is no need to fix the axis of the inner cannula with respect to the curved outer cannula. This allows simpler inner cannula mounts that may be front or back-loaded, without requiring an access door to the hand piece chamber. This design thus allows cheaper manufacturing, easier tolerancing, less expensive materials, and advantages in the size and complexity of cannula mounts.

Alternative Embodiments which Readily Come to Mind

While the twin-cannula assemblies shown in the illustrative embodiments have been shown used with a twin cannula assembly, it is understood that further alternate embodiments will readily come to mind in view of the present invention disclosure.

For example, while the cross-sectional dimensions of the inner cannula guide tube 105 of the illustrative embodiments has been disclosed as being circular, it is understood that the cross-sectional dimension be oval, square or other geometry, which will ensure axial alignment of the inner cannula within the outer cannula.

When constructing RF-based bipolar electro-cauterizing twin-cannula assemblies according to the present invention, there are various ways of supplying electrical RF power to the moving inner cannula. For example, one way is to provide a traveling RF-cautery power supply wire that delivers RF power to the moving inner cannula base portion, rather than a bushing in direct physical contact with an uncoated portion of the electrically-conductive inner cannula which will inevitably be vulnerable to rapid wear.

Reverse-wired electromagnetic coils, and/or MuMetal windings can be used at each pole in the stationary electromagnetic coil structure within the hand-piece, to increase the flux at those poles and thus increase stroke power. Rare-earth high permeability permanent magnets can be used to increase the magnetic flux, and thus magnetic force field, at those poles and thus increase stroke power. Also, a pair of axially-polarized ring magnets can be arranged as SNNS or NSSN to augment the central pole flux on the moving inner cannula base portion 94, which supports the permanent ring magnet 94D, which subassembly functions as an inner cannula actuator.

While the particular embodiments shown and described above have proven to be useful in many applications in the liposuction art, further modifications of the present invention disclosed herein will occur to persons skilled in the art to which the present invention pertains. All such modifications are deemed to be within the scope and spirit of the present invention defined by the appended Claims.

1. A tissue aspiration instrumentation system comprising:
a hand-supportable tissue aspiration instrument including
a hand-supportable housing having
a front portion and a rear portion aligned along a longitudinal axis,
an interior volume; and
a cannula drive mechanism disposed within said interior volume; and
a twin cannula assembly having
a hollow inner cannula with an open-end type opening and having an hollow inner cannula base portion; and
wherein said hollow outer cannula has multiple outer aspiration apertures formed about the distal portion of said hollow outer cannula, and an outer cannula base portion stationarily connected to the front portion of said hand-supportable housing; and
wherein said cannula drive mechanism causes (i) said hollow inner cannula base portion to reciprocate within said interior volume, (ii) said hollow inner cannula to reciprocate within said hollow outer cannula, and (iii) said open-end ending to reciprocate along said distal portion of said hollow outer cannula, while tissue is being aspirated through said multiple outer aspiration apertures and through said reciprocating open-end type aspiration opening, and along a fluid communication channel extending from said open-end type aspiration opening, along said hollow inner cannula and said hollow inner cannula base portion through and through a section of flexible tubing connected to said vacuum source.

2. The tissue aspiration instrument of claim 1,
wherein said hollow inner cannula base portion comprises a tubular structure having a permanent magnet ring mounted about its outer surface and concentric with the longitudinal axis of said hollow inner cannula; and
wherein said cannula drive mechanism comprises at least one electromagnetic wire coil wound about a cylindrical guide tube installed in said interior volume, and for generating an electromagnetic force field that is driven by an electrical signal source, and electrically connected to an electrical signal source, for generating an electromagnetic force field which periodically pushes and pulls said permanent magnet ring and thereby causes (i) said hollow inner cannula base portion to reciprocate within said cylindrical guide tube, (ii) said hollow inner cannula to reciprocate within said hollow outer cannula, and said inner aspiration aperture to reciprocate along said elongated outer aspiration aperture.

3. The tissue aspiration instrument of claim 1 wherein said permanent magnet ring and said at least one electromagnetic coil form a magnetic coupling mechanism between said hollow inner cannula base portion and said cylindrical guide tube.

4. The tissue aspiration instrument of claim 2, wherein a stationary tubing connector is provided on the rear portion of said housing, and said stationary tubing connector comprises a barb-type connector to receiving and gripping said end portion of said flexible aspiration tubing.

5. The tissue aspiration instrument of claim 1, wherein said stationary tubing connector is provided on the rear portion of said housing, and said stationary tubing connector comprises a snap-lock type connector for establishing and maintaining a connection with said end portion of flexible aspiration tubing.

6. The tissue aspiration instrument of claim 1, wherein said hollow inner cannula base portion comprises is operably connected with said cannula drive mechanism, and reciprocates said hollow inner cannula base portion within said interior volume.

7. The tissue aspiration instrument of claim 1 wherein a tubing connector is provided on hollow inner cannula, for receiving and gripping said end portion of said flexible aspiration tubing.

8. The tissue aspiration instrument of claim 1, wherein said multiple outer aspiration apertures comprise multiple elongated outer aspiration apertures formed about the distal portion of said hollow outer cannula.

9. The tissue aspiration instrument of claim 1,
wherein said multiple outer aspiration apertures comprise first, second and third groups of outer aspiration apertures formed about the distal portion of said hollow outer cannula;
wherein said first group of three elongated outer aspiration apertures closest to the distal end of said hollow outer cannula is designated as zone 3, said second group of outer aspiration apertures closest to the proximal end of said hollow outer cannula is designated as zone 1, and said second group of outer aspiration apertures between zone 1 and zone 3 is designated as zone 2; and
wherein the open-end type opening of said hollow inner cannula travels between said zone 1 and zone 3, during each forward-stroke and back-stroke of said hollow inner cannula.

10. A twin cannula assembly for use with a tissue aspiration instrumentation system including a hand-supportable tissue aspiration instrument including a hand-supportable housing having a front portion and a rear portion aligned along a longitudinal axis, an interior volume, and a cannula drive mechanism disposed within said interior volume, wherein said twin cannula assembly comprises:
a hollow inner cannula with an open-end type aspiration opening, and having an hollow inner cannula base portion; and
wherein said hollow outer cannula has multiple elongated outer aspiration apertures about its distal portion, and an outer cannula base portion stationarily connected to the front portion of said hand-supportable housing.

11. The twin cannula assembly of claim 10, wherein said multiple outer aspiration apertures comprise multiple elongated outer aspiration apertures formed about the distal portion of said hollow outer cannula.

12. The tissue aspiration instrument of claim 10,
wherein said multiple outer aspiration apertures comprise first, second and third groups of outer aspiration apertures formed about the distal portion of said hollow outer cannula;
wherein said first group of outer aspiration apertures closest to the distal end of said hollow outer cannula is designated as zone 3, said second group aspiration apertures closest to the proximal end of said hollow outer cannula is designated as zone 1, and said second group of outer aspiration apertures between zone 1 and zone 3 is designated as zone 2; and
wherein said open-end type aspiration opening travels between said zone 1 and zone 3, during each forward-stroke and back-stroke of said open-ended inner cannula.

13. A power-assisted tissue-aspiration instrumentation system comprising:
a hand-supportable housing having
a front portion and a rear portion aligned along a longitudinal axis,
an interior volume; and
a cannula drive mechanism disposed within said interior volume; and
a twin cannula assembly having
a hollow inner cannula with an open-end type aspiration opening and having an hollow inner cannula base portion;
wherein said hollow inner cannula is disposed in said disposed within said hollow outer cannula; and
wherein said hollow outer cannula has multiple outer aspiration apertures formed about the distal portion of said hollow outer cannula, and an outer cannula base portion stationarily connected to the front portion of said hand-supportable housing;
wherein said multiple outer aspiration apertures comprise first, second and third groups of outer aspiration apertures formed about the distal portion of said hollow outer cannula;
wherein said first group of outer aspiration apertures is formed closest to the distal end of said hollow outer cannula, said second group of outer aspiration apertures is formed closest to the proximal end of said hollow outer cannula, and said second group of outer aspiration apertures is formed said first and third outer aspiration apertures; and
wherein during system operation, said cannula drive mechanism causes (i) said hollow inner cannula base portion to reciprocate within said interior volume, (ii) said hollow inner cannula to reciprocate within said hollow outer cannula, and (iii) said open-end type aspiration opening to reciprocate back and forth to a mid position between said first group of aspiration apertures and said third group of outer aspiration apertures, so that vacuum pressure is always delivered to at least ½ of one said outer aspiration aperture groups as said hollow inner cannula is reciprocated back and forward within said hollow outer cannula, cutting off fat being aspirated into said hollow inner cannula lumen, and thereby progressively delivering more suction performance and achieving a scissoring-effect during tissue aspiration operations.

14. (canceled)
